SEQUENCE LISTING (575 sequences)

SEQ ID NO: 1 - DNA, 161 bp, synthetic construct
  misc_feature 1..161: note = source = /note="Description of Artificial Sequence: Synthetic polynucleotide"
  source 1..161: mol_type = other DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctg

SEQ ID NO: 2 - DNA, 127 bp, synthetic construct
  misc_feature 1..127: note = source = /note="Description of Artificial Sequence: Synthetic polynucleotide"
  source 1..127: mol_type = other DNA
ctggggcaaaccagcgtggaccgcttgctgcaactctctcagggccaggcggtgaagggcaatcagctgttgcccgtctcactggtgaaaagaaaaaccaccctggcgcccaatacgcaaaccgcct

SEQ ID NO: 3 - DNA, 25 bp, synthetic construct
  misc_feature 1..25: note = source = /note="Description of Artificial Sequence: Synthetic oligonucleotide"
  source 1..25: mol_type = other DNA
ggtttgccccagcaggcgaaaatcc

SEQ ID NO: 4 - DNA, 18 bp, synthetic construct
  misc_feature 1..18: note = source = /note="Description of Artificial Sequence: Synthetic oligonucleotide"
  source 1..18: mol_type = other DNA
caccagtgagacggattg

SEQ ID NO: 5 - DNA, 18 bp, synthetic construct
  misc_feature 1..18: note = source = /note="Description of Artificial Sequence: Synthetic oligonucleotide"
  source 1..18: mol_type = other DNA
agtccactattaaatcca

SEQ ID NO: 6 - DNA, 18 bp, synthetic construct
  misc_feature 1..18: note = source = /note="Description of Artificial Sequence: Synthetic oligonucleotide"
  source 1..18: mol_type = other DNA
atcctgtttgatggatcg

SEQ ID NO: 7 - DNA, 47 bp, synthetic construct
  misc_feature 1..47: note = source = /note="Description of Artificial Sequence: Synthetic oligonucleotide"
  source 1..47: mol_type = other DNA
gcaaaaatcaaaagaattgcccgagatagggggcgacaaagttgagt

SEQ ID NO: 8 - DNA, 47 bp, synthetic construct
  misc_feature 1..47: note = source = /note="Description of Artificial Sequence: Synthetic oligonucleotide"
  source 1..47: mol_type = other DNA
acgtaaaaccgtctatttaggcggtttgcgtgcctgtcaccattggg

SEQ ID NO: 9 - DNA, 47 bp, synthetic construct
  misc_feature 1..47: note = source = /note="Description of Artificial Sequence: Synthetic oligonucleotide"
  source 1..47: mol_type = other DNA
ccctgccctgagagagttgcagcaagcggtccttataatcccacgct

SEQ ID NO: 10 - DNA, 37 bp, synthetic construct
  misc_feature 1..37: note = source = /note="Description of Artificial Sequence: Synthetic oligonucleotide"
  source 1..37: mol_type = other DNA
cgccagggttttggacgaacgttttggtttttctttt

SEQ ID NO: 11 - DNA, 37 bp, synthetic construct
  misc_feature 1..37: note = source = /note="Description of Artificial Sequence: Synthetic oligonucleotide"
  source 1..37: mol_type = other DNA
gttgttcctttccgaatggtttttagtttggaacaag

SEQ ID NO: 12 - DNA, 37 bp, synthetic construct
  misc_feature 1..37: note = source = /note="Description of Artificial Sequence: Synthetic oligonucleotide"
  source 1..37: mol_type = other DNA
ggtttgcctttagctggcaactttccagcaggcgaaa

SEQ ID NO: 13 - DNA, 7249 bp, M13 bacteriophage
  source 1..7249: mol_type = genomic DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 14 - DNA, 8462 bp, PhiX174 bacteriophage
  source 1..8462: mol_type = genomic DNA
gagttttatcgcttccatgacgcagaagttaacactttcggatatttctgatgagtcgaaaaattatcttgataaagcaggaattactactgcttgtttacgaattaaatcgaagtggactgctggcggaaaatgagaaaattcgacctatccttgcgcagctcgagaagctcttactttgcgacctttcgccatcaactaacgattctgtcaaaaactgacgcgttggatgaggagaagtggcttaatatgcttggcacgttcgtcaaggactggtttagatatgagtcacattttgttcatggtagagattctcttgttgacattttaaaagagcgtggattactatctgagtccgatgctgttcaaccactaataggtaagaaatcatgagtcaagttactgaacaatccgtacgtttccagaccgctttggcctctattaagctcattcaggcttctgccgttttggatttaaccgaagatgatttcgattttctgacgagtaacaaagtttggattgctactgaccgctctcgtgctcgtcgctgcgttgaggcttgcgtttatggtacgctggactttgtaggataccctcgctttcctgctcctgttgagtttattgctgccgtcattgcttattatgttcatcccgtcaacattcaaacggcctgtctcatcatggaaggcgctgaatttacggaaaacattattaatggcgtcgagcgtccggttaaagccgctgaattgttcgcgtttaccttgcgtgtacgcgcaggaaacactgacgttcttactgacgcagaagaaaacgtgcgtcaaaaattacgtgcagaaggagtgatgtaatgtctaaaggtaaaaaacgttctggcgctcgccctggtcgtccgcagccgttgcgaggtactaaaggcaagcgtaaaggcgctcgtctttggtatgtaggtggtcaacaattttaattgcaggggcttcggccccttacttgaggataaattatgtctaatattcaaactggcgccgagcgtatgccgcatgacctttcccatcttggcttccttgctggtcagattggtcgtcttattaccatttcaactactccggttatcgctggcgactccttcgagatggacgccgttggcgctctccgtctttctccattgcgtcgtggccttgctattgactctactgtagacatttttactttttatgtccctcatcgtcacgtttatggtgaacagtggattaagttcatgaaggatggtgttaatgccactcctctcccgactgttaacactactggttatattgaccatgccgcttttcttggcacgattaaccctgataccaataaaatccctaagcatttgtttcagggttatttgaatatctataacaactattttaaagcgccgtggatgcctgaccgtaccgaggctaaccctaatgagcttaatcaagatgatgctcgttatggtttccgttgctgccatctcaaaaacatttggactgctccgcttcctcctgagactgagctttctcgccaaatgacgacttctaccacatctattgacattatgggtctgcaagctgcttatgctaatttgcatactgaccaagaacgtgattacttcatgcagcgttaccatgatgttatttcttcatttggaggtaaaacctcttatgacgctgacaaccgtcctttacttgtcatgcgctctaatctctgggcatctggctatgatgttgatggaactgaccaaacgtcgttaggccagttttctggtcgtgttcaacagacctataaacattctgtgccgcgtttctttgttcctgagcatggcactatgtttactcttgcgcttgttcgttttccgcctactgcgactaaagagattcagtaccttaacgctaaaggtgctttgacttataccgatattgctggcgaccctgttttgtatggcaacttgccgccgcgtgaaatttctatgaaggatgttttccgttctggtgattcgtctaagaagtttaagattgctgagggtcagtggtatcgttatgcgccttcgtatgtttctcctgcttatcaccttcttgaaggcttcccattcattcaggaaccgccttctggtgatttgcaagaacgcgtacttattcgccaccatgattatgaccagtgtttccagtccgttcagttgttgcagtggaatagtcaggttaaatttaatgtgaccgtttatcgcaatctgccgaccactcgcgattcaatcatgacttcgtgataaaagattgagtgtgaggttataacgccgaagcggtaaaaattttaatttttgccgctgaggggttgaccaagcgaagcgcggtaggttttctgcttaggagtttaatcatgtttcagacttttatttctcgccataattcaaactttttttctgataagctggttctcacttctgttactccagcttcttcggcacctgttttacagacacctaaagctacatcgtcaacgttatattttgatagtttgacggttaatgctggtaatggtggttttcttcattgcattcagatggatacatctgtcaacgccgctaatcaggttgtttctgttggtgctgatattgcttttgatgccgaccctaaattttttgcctgtttggttcgctttgagtcttcttcggttccgactaccctcccgactgcctatgatgtttatcctttggatggtcgccatgatggtggttattataccgtcaaggactgtgtgactattgacgtccttccccgtacgccgggcaataatgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtcaaaaagccgcctccggtggcattcaaggtgatgtgcttgctaccgataacaatactgtaggcatgggtgagagttttatcgcttccatgacgcagaagttaacactttcggatatttctgatgagtcgaaaaattatcttgataaagcaggaattactactgcttgtttacgaattaaatcgaagtggactgctggcggaaaatgagaaaattcgacctatccttgcgcagctcgagaagctcttactttgcgacctttcgccatcaactaacgattctgtcaaaaactgacgcgttggatgaggagaagtggcttaatatgcttggcacgttcgtcaaggactggtttagatatgagtcacattttgttcatggtagagattctcttgttgacattttaaaagagcgtggattactatctgagtccgatgctgttcaaccactaataggtaagaaatcatgagtcaagttactgaacaatccgtacgtttccagaccgctttggcctctattaagctcattcaggcttctgccgttttggatttaaccgaagatgatttcgattttctgacgagtaacaaagtttggattgctactgaccgctctcgtgctcgtcgctgcgttgaggcttgcgtttatggtacgctggactttgtaggataccctcgctttcctgctcctgttgagtttattgctgccgtcattgcttattatgttcatcccgtcaacattcaaacggcctgtctcatcatggaaggcgctgaatttacggaaaacattattaatggcgtcgagcgtccggttaaagccgctgaattgttcgcgtttaccttgcgtgtacgcgcaggaaacactgacgttcttactgacgcagaagaaaacgtgcgtcaaaaattacgtgcagaaggagtgatgtaatgtctaaaggtaaaaaacgttctggcgctcgccctggtcgtccgcagccgttgcgaggtactaaaggcaagcgtaaaggcgctcgtctttggtatgtaggtggtcaacaattttaattgcaggggcttcggccccttacttgaggataaattatgtctaatattcaaactggcgccgagcgtatgccgcatgacctttcccatcttggcttccttgctggtcagattggtcgtcttattaccatttcaactactccggttatcgctggcgactccttcgagatggacgccgttggcgctctccgtctttctccattgcgtcgtggccttgctattgactctactgtagacatttttactttttatgtccctcatcgtcacgtttatggtgaacagtggattaagttcatgaaggatggtgttaatgccactcctctcccgactgttaacactactggttatattgaccatgccgcttttcttggcacgattaaccctgataccaataaaatccctaagcatttgtttcagggttatttgaatatctataacaactattttaaagcgccgtggatgcctgaccgtaccgaggctaaccctaatgagcttaatcaagatgatgctcgttatggtttccgttgctgccatctcaaaaacatttggactgctccgcttcctcctgagactgagctttctcgccaaatgacgacttctaccacatctattgacattatgggtctgcaagctgcttatgctaatttgcatactgaccaagaacgtgattacttcatgcagcgttaccatgatgttatttcttcatttggaggtaaaacctcttatgacgctgacaaccgtcctttacttgtcatgcgctctaatctctgggcatctggctatgatgttgatggaactgaccaaacgtcgttaggccagttttctggtcgtgttcaacagacctataaacattctgtgccgcgtttctttgttcctgagcatggcactatgtttactcttgcgcttgttcgttttccgcctactgcgactaaagagattcagtaccttaacgctaaaggtgctttgacttataccgatattgctggcgaccctgttttgtatggcaacttgccgccgcgtgaaatttctatgaaggatgttttccgttctggtgattcgtctaagaagtttaagattgctgagggtcagtggtatcgttatgcgccttcgtatgtttctcctgcttatcaccttcttgaaggcttcccattcattcaggaaccgccttctggtgatttgcaagaacgcgtacttattcgccaccatgattatgaccagtgtttccagtccgttcagttgttgcagtggaatagtcaggttaaatttaatgtgaccgtttatcgcaatctgccgaccactcgcgattcaatcatgacttcgtgataaaagattgagtgtgaggttataacgccgaagcggtaaaaattttaatttttgccgctgaggggttgaccaagcgaagcgcggtaggttttctgcttaggagtttaatcatgtttcagacttttatttctcgccataattcaaactttttttctgataagctggttctcacttctgttactccagcttcttcggcacctgttttacagacacctaaagctacatcgtcaacgttatattttgatagtttgacggttaatgctggtaatggtggttttcttcattgcattcagatggatacatctgtcaacgccgctaatcaggttgtttctgttggtgctgatattgcttttgatgccgaccctaaattttttgcctgtttggttcgctttgagtcttcttcggttccgactaccctcccgactgcctatgatgtttatcctttggatggtcgccatgatggtggttattataccgtcaaggactgtgtgactattgacgtccttccccgtacgccgggcaataatgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtcaaaaagccgcctccggtggcattcaaggtgatgtgcttgctaccgataacaatactgtaggcatgggtgatgctggtattaaatctgccattcaaggctctaatgttcctaaccctgatgaggccgtccctagttttgtttctggtgctatggctaaagctggtaaaggacttcttgaaggtacgttgcaggctggcacttctgccgtttctgataagttgcttgatttggttggacttggtggcaagtctgccgctgataaaggaaaggatactcgtgattatcttgctgctgcatttcctgagcttaatgcttgggagcgtgctggtgctgatgcttcctctgctggtatggttgacgccggatttgagaatcaaaaagagcttactaaaatgcaactggacaatcagaaagagattgccgagatgcaaaatgagactcaaaaagagattgctggcattcagtcggcgacttcacgccagaatacgaaagaccaggtatatgcacaaaatgagatgcttgcttatcaacagaaggagtctactgctcgcgttgcgtctattatggaaaacaccaatctttccaagcaacagcaggtttccgagattatgcgccaaatgcttactcaagctcaaacggctggtcagtattttaccaatgaccaaatcaaagaaatgactcgcaaggttagtgctgaggttgacttagttcatcagcaaacgcagaatcagcggtatggctcttctcatattggcgctactgcaaaggatatttctaatgtcgtcactgatgctgcttctggtgtggttgatatttttcatggtattgataaagctgttgccgatacttggaacaatttctggaaagacggtaaagctgatggtattggctctaatttgtctaggaaataaccgtcaggattgacaccctcccaattgtatgttttcatgcctccaaatcttggaggcttttttatggttcgttcttattacccttctgaatgtcacgctgattattttgactttgagcgtatcgaggctcttaaacctgctattgaggcttgtggcatttctactctttctcaatccccaatgcttggcttccataagcagatggataaccgcatcaagctcttggaagagattctgtcttttcgtatgcagggcgttgagttcgataatggtgatatgtatgttgacggccataaggctgcttctgacgttcgtgatgagtttgtatctgttactgagaagttaatggatgaattggcacaatgctacaatgtgctcccccaacttgatattaataacactatagaccaccgccccgaaggggacgaaaaatggtttttagagaacgagaagacggttacgcagttttgccgcaagctggctgctgaacgccctcttaaggatattcgcgatgagtataattaccccaaaaagaaaggtattaaggatgagtgttcaagattgctggaggcctccactatgaaatcgcgtagaggctttgctattcagcgtttgatgaatgcaatgcgacaggctcatgctgatggttggtttatcgtttttgacactctcacgttggctgacgaccgattagaggcgttttatgataatcccaatgctttgcgtgactattttcgtgatattggtcgtatggttcttgctgccgagggtcgcaaggctaatgattcacacgccgactgctatcagtatttttgtgtgcctgagtatggtacagctaatggccgtcttcatttccatgcggtgcactttatgcggacacttcctacaggtagcgttgaccctaattttggtcgtcgggtacgcaatcgccgccagttaaatagcttgcaaaatacgtggccttatggttacagtatgcccatcgcagttcgctacacgcaggacgctttttcacgttctggttggttgtggcctgttgatgctaaaggtgagccgcttaaagctaccagttatatggctgttggtttctatgtggctaaatacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggttacgacgcgacgccgttcaaccagatattgaagcagaacgcaaaaagagagatgagattgaggctgggaaaagttactgtagccgacgttttggcggcgcaacctgtgacgacaaatctgctcaaatttatgcgcgcttcgataaaaatgattggcgtatccaacctgca

SEQ ID NO: 15 - DNA, 31 bp, synthetic construct
  misc_feature 1..31: note = source = /note="Description of Artificial Sequence: Synthetic oligonucleotide"
  source 1..31: mol_type = other DNA
agtaagcagatagctcagagcaggcgaacaa

SEQ ID NO: 16 - DNA, 34 bp, synthetic construct
  misc_feature 1..34: note = source = /note="Description of Artificial Sequence: Synthetic oligonucleotide"
  source 1..34: mol_type = other DNA
gaaatagctaataacggaagagcccaccataccc

SEQ ID NO: 17 - DNA, 44 bp, synthetic construct
  misc_feature 1..44: note = source = /note="Description of Artificial Sequence: Synthetic oligonucleotide"
  source 1..44: mol_type = other DNA
agtttacatacataaaggtggcaacataagaaccacgccgccag

SEQ ID NO: 18 - DNA, 21 bp, synthetic construct
  misc_feature 1..21: note = source = /note="Description of Artificial Sequence: Synthetic oligonucleotide"
  source 1..21: mol_type = other DNA
aaacgcaaaatagctaagaaa

SEQ ID NO: 19 - DNA, 32 bp, synthetic construct
  misc_feature 1..32: note = source = /note="Description of Artificial Sequence: Synthetic oligonucleotide"
  source 1..32: mol_type = other DNA
aggttgagcgattggcaaatcctcataacaat

SEQ ID NO: 20 - DNA, 30 bp, synthetic construct
  misc_feature 1..30: note = source = /note="Description of Artificial Sequence: Synthetic oligonucleotide"
  source 1..30: mol_type = other DNA
aaaagaactggcatgtagaaaaaccagaag

SEQ ID NO: 21 - DNA, 34 bp, synthetic construct
  misc_feature 1..34: note = source = /note="Description of Artificial Sequence: Synthetic oligonucleotide"
  source 1..34: mol_type = other DNA
tagcgtttccctcagagcctaatccaccgaccac

SEQ ID NO: 22 - DNA, 44 bp, synthetic construct
  misc_feature 1..44: note = source = /note="Description of Artificial Sequence: Synthetic oligonucleotide"
  source 1..44: mol_type = other DNA
ccctgcaaagacaccacggaataagtttgatagcagagtagcga

SEQ ID NO: 23 - DNA, 30 bp, synthetic construct
  misc_feature 1..30: note = source = /note="Description of Artificial Sequence: Synthetic oligonucleotide"
  source 1..30: mol_type = other DNA
cctcagagccgccaaaagaaaccagagccg

SEQ ID NO: 24 - DNA, 31 bp, synthetic construct
  misc_feature 1..31: note = source = /note="Description of Artificial Sequence: Synthetic oligonucleotide"
  source 1..31: mol_type = other DNA
gagccaccaccggatcagatagcgaccgcct

SEQ ID NO: 25 - DNA, 21 bp, synthetic construct
  misc_feature 1..21: note = source = /note="Description of Artificial Sequence: Synthetic oligonucleotide"
  source 1..21: mol_type = other DNA
aaccgccagccatcttaacca

SEQ ID NO: 26 - DNA, 32 bp, synthetic construct
  misc_feature 1..32: note = source = /note="Description of Artificial Sequence: Synthetic oligonucleotide"
  source 1..32: mol_type = other DNA
tttgccttctgtagcggtcatagcccccttat

SEQ ID NO: 27 - DNA, 28 bp, synthetic construct
  misc_feature 1..28: note = source = /note="Description of Artificial Sequence: Synthetic oligonucleotide"
  source 1..28: mol_type = other DNA
attctgaaaccatttgtcacattagcaa

SEQ ID NO: 28 - DNA, 44 bp, synthetic construct
  misc_feature 1..44: note = source = /note="Description of Artificial Sequence: Synthetic oligonucleotide"
  source 1..44: mol_type = other DNA
taccaatcaatacgcagtatgttagcaattaagactccaaagac

SEQ ID NO: 29 - DNA, 21 bp, synthetic construct
  misc_feature 1..21: note = source = /note="Description of Artificial Sequence: Synthetic oligonucleotide"
  source 1..21: mol_type = other DNA
gtcaccaacaccgactgccag

SEQ ID NO: 30 - DNA, 35 bp, synthetic construct
  misc_feature 1..35: note = source = /note="Description of Artificial Sequence: Synthetic oligonucleotide"
  source 1..35: mol_type = other DNA
cattcaacagggaaggtcattaaaggtgaattatc

SEQ ID NO: 31 - DNA, 31 bp, synthetic construct
  misc_feature 1..31: note = source = /note="Description of Artificial Sequence: Synthetic oligonucleotide"
  source 1..31: mol_type = other DNA
caaaatcaccagtagagggcgattgcaccat

SEQ ID NO: 32 - DNA, 33 bp, synthetic construct
  misc_feature 1..33: note = source = /note="Description of Artificial Sequence: Synthetic oligonucleotide"
  source 1..33: mol_type = other DNA
accgtatatggtttaccagcgccttattagaaa

SEQ ID NO: 33 - DNA, 44 bp, synthetic construct
  misc_feature 1..44: note = source = /note="Description of Artificial Sequence: Synthetic oligonucleotide"
  source 1..44: mol_type = other DNA
gacttatagcccccacagacagccctcacaacgcctggaggttt

SEQ ID NO: 34 - DNA, 32 bp, synthetic construct
  misc_feature 1..32: note = source = /note="Description of Artificial Sequence: Synthetic oligonucleotide"
  source 1..32: mol_type = other DNA
ccctcagatcagaaccgaaccgaacttttgat

SEQ ID NO: 35 - DNA, 21 bp, synthetic construct
  misc_feature 1..21: note = source = /note="Description of Artificial Sequence: Synthetic oligonucleotide"
  source 1..21: mol_type = other DNA
gattagcgagtgtactgaaac

SEQ ID NO: 36 - DNA, 36 bp, synthetic construct
  misc_feature 1..36: note = source = /note="Description of Artificial Sequence: Synthetic oligonucleotide"
  source 1..36: mol_type = other DNA
gatacaggtgtatcaccgtactcagtagcattggaa

SEQ ID NO: 37 - DNA, 31 bp, synthetic construct
  misc_feature 1..31: note = source = /note="Description of Artificial Sequence: Synthetic oligonucleotide"
  source 1..31: mol_type = other DNA
atgaaagtattaagcacccaccgcaggctga

SEQ ID NO: 38 - DNA, 28 bp, synthetic construct
  misc_feature 1..28: note = source = /note="Description of Artificial Sequence: Synthetic oligonucleotide"
  source 1..28: mol_type = other DNA
tagggggttttggatataagcctcaaga

SEQ ID NO: 39 - DNA, 28 bp, synthetic construct
  misc_feature 1..28: note = source = /note="Description of Artificial Sequence: Synthetic oligonucleotide"
  source 1..28: mol_type = other DNA
ttttgtacaaacgttagcgttaccgtaa

SEQ ID NO: 40 - DNA, 31 bp, synthetic construct
  misc_feature 1..31: note = source = /note="Description of Artificial Sequence: Synthetic oligonucleotide"
  source 1..31: mol_type = other DNA
atagcaagcccaatctaaattttgaggaacc

SEQ ID NO: 41 - DNA, 36 bp, synthetic construct
  misc_feature 1..36: note = source = /note="Description of Artificial Sequence: Synthetic oligonucleotide"
  source 1..36: mol_type = other DNA
ccatgttagtcgtctttccagacgtttaattgaaag

SEQ ID NO: 42 - DNA, 44 bp, synthetic construct
  misc_feature 1..44: note = source = /note="Description of Artificial Sequence: Synthetic oligonucleotide"
  source 1..44: mol_type = other DNA
catgaacgatcttatcggtttatcagctaaggagccttagtaaa

SEQ ID NO: 43 - DNA, 21 bp, synthetic construct
  misc_feature 1..21: note = source = /note="Description of Artificial Sequence: Synthetic oligonucleotide"
  source 1..21: mol_type = other DNA
cgtcaccacttagccgcaggg

SEQ ID NO: 44 - DNA, 32 bp, synthetic construct
  misc_feature 1..32: note = source = /note="Description of Artificial Sequence: Synthetic oligonucleotide"
  source 1..32: mol_type = other DNA
gtatgggacaactttcaaatccgcgacctgct

SEQ ID NO: 45 - DNA, 36 bp, synthetic construct
  misc_feature 1..36: note = source = /note="Description of Artificial Sequence: Synthetic oligonucleotide"
  source 1..36: mol_type = other DNA
gccactacgttaatgctgagtaacagtgcccgaggc

SEQ ID NO: 46 - DNA, 28 bp, synthetic construct
  misc_feature 1..28: note = source = /note="Description of Artificial Sequence: Synthetic oligonucleotide"
  source 1..28: mol_type = other DNA
ctcagcaggaatacacaaggccgcaacg

SEQ ID NO: 47 - DNA, 32 bp, synthetic construct
  misc_feature 1..32: note = source = /note="Description of Artificial Sequence: Synthetic oligonucleotide"
  source 1..32: mol_type = other DNA
aaaaaggaagtttccattaaggcggatatttt

SEQ ID NO: 48 - DNA, 44 bp, synthetic construct
  misc_feature 1..44: note = source = /note="Description of Artificial Sequence: Synthetic oligonucleotide"
  source 1..44: mol_type = other DNA
ctaataaagactagtgccgtcgagagggcagtaccaacgggtaa

SEQ ID NO: 49 - DNA, 24 bp, synthetic construct
  misc_feature 1..24: note = source = /note="Description of Artificial Sequence: Synthetic oligonucleotide"
  source 1..24: mol_type = other DNA
aaagtataaacagaaggcaccaac

SEQ ID NO: 50 - DNA, 28 bp, synthetic construct
  misc_feature 1..28: note = source = /note="Description of Artificial Sequence: Synthetic oligonucleotide"
  source 1..28: mol_type = other DNA
catgcgaaagacttgaggacttttgcgg

SEQ ID NO: 51 - DNA, 44 bp, synthetic construct
  misc_feature 1..44: note = source = /note="Description of Artificial Sequence: Synthetic oligonucleotide"
  source 1..44: mol_type = other DNA
aattgtgaatttgcaacggctacagaggcatcggaatgcgccga

SEQ ID NO: 52 - DNA, 32 bp, synthetic construct
  misc_feature 1..32: note = source = /note="Description of Artificial Sequence: Synthetic oligonucleotide"
  source 1..32: mol_type = other DNA
aaccatcgaccgatattataccaagcgcgaaa

SEQ ID NO: 53 - DNA, 28 bp, synthetic construct
  misc_feature 1..28: note = source = /note="Description of Artificial Sequence: Synthetic oligonucleotide"
  source 1..28: mol_type = other DNA
aacaaaggctccctttcgagttttcacg

SEQ ID NO: 54 - DNA, 21 bp, synthetic construct
  misc_feature 1..21: note = source = /note="Description of Artificial Sequence: Synthetic oligonucleotide"
  source 1..21: mol_type = other DNA
ccaaaaaaaacggagagaaca

SEQ ID NO: 55 - DNA, 31 bp, synthetic construct
  misc_feature 1..31: note = source = /note="Description of Artificial Sequence: Synthetic oligonucleotide"
  source 1..31: mol_type = other DNA
actaaaggaattgcgcatacccacgaataat

SEQ ID NO: 56 - DNA, 36 bp, synthetic construct
  misc_feature 1..36: note = source = /note="Description of Artificial Sequence: Synthetic oligonucleotide"
  source 1..36: mol_type = other DNA
caaagtacgcttgataccgatagtcgagggtactta

SEQ ID NO: 57 - DNA, 25 bp, synthetic construct
  misc_feature 1..25: note = source = /note="Description of Artificial Sequence: Synthetic oligonucleotide"
  source 1..25: mol_type = other DNA
tgtatcatcgcctgataaattgtgt

SEQ ID NO: 58 - DNA, 25 bp, synthetic construct
  misc_feature 1..25: note = source = /note="Description of Artificial Sequence: Synthetic oligonucleotide"
  source 1..25: mol_type = other DNA
aaacactcatctttgacccccagcg

SEQ ID NO: 59 - DNA, 25 bp, synthetic construct
  misc_feature 1..25: note = source = /note="Description of Artificial Sequence: Synthetic oligonucleotide"
  source 1..25: mol_type = other DNA
caccctcagagccaccaccctcatt

SEQ ID NO: 60 - DNA, 25 bp, synthetic construct
  misc_feature 1..25: note = source = /note="Description of Artificial Sequence: Synthetic oligonucleotide"
  source 1..25: mol_type = other DNA
cctgcctatttcggaacctattatt

SEQ ID NO: 61 - DNA, 25 bp, synthetic construct
  misc_feature 1..25: note = source = /note="Description of Artificial Sequence: Synthetic oligonucleotide"
  source 1..25: mol_type = other DNA
cagtttcagcggagtgagaatagaa

SEQ ID NO: 62 - DNA, 25 bp, synthetic construct
  misc_feature 1..25: note = source = /note="Description of Artificial Sequence: Synthetic oligonucleotide"
  source 1..25: mol_type = other DNA
taataagttttaacggggtcagtgc

SEQ ID NO: 63 - DNA, 25 bp, synthetic construct
  misc_feature 1..25: note = source = /note="Description of Artificial Sequence: Synthetic oligonucleotide"
  source 1..25: mol_type = other DNA
acgaggcgcagacggtcaatcataa

SEQ ID NO: 64 - DNA, 25 bp, synthetic construct
  misc_feature 1..25: note = source = /note="Description of Artificial Sequence: Synthetic oligonucleotide"
  source 1..25: mol_type = other DNA
tcggtcgctgaggcttgcagggagt

SEQ ID NO: 65 - DNA, 36 bp, synthetic construct
  misc_feature 1..36: note = source = /note="Description of Artificial Sequence: Synthetic oligonucleotide"
  source 1..36: mol_type = other DNA
cttgccctactaatgcagatacatatagcgtccaca

SEQ ID NO: 66 - DNA, 21 bp, synthetic construct
  misc_feature 1..21: note = source = /note="Description of Artificial Sequence: Synthetic oligonucleotide"
  source 1..21: mol_type = other DNA
aataaaacgacgagaaaactg

SEQ ID NO: 67 - DNA, 28 bp, synthetic construct
  misc_feature 1..28: note = source = /note="Description of Artificial Sequence: Synthetic oligonucleotide"
  source 1..28: mol_type = other DNA
ttcagaactaactgagatttggaagaaa

SEQ ID NO: 68 - DNA, 32 bp, synthetic construct
  misc_feature 1..32: note = source = /note="Description of Artificial Sequence: Synthetic oligonucleotide"
  source 1..32: mol_type = other DNA
gaggcatacactatcaatatgtacccataagg

SEQ ID NO: 69 - DNA, 44 bp, synthetic construct
  misc_feature 1..44: note = source = /note="Description of Artificial Sequence: Synthetic oligonucleotide"
  source 1..44: mol_type = other DNA
gttgaggaataccaatactgcggaatcgtagactggaacgccaa

SEQ ID NO: 70 - DNA, 31 bp, synthetic construct
  misc_feature 1..31: note = source = /note="Description of Artificial Sequence: Synthetic oligonucleotide"
  source 1..31: mol_type = other DNA
gctcattataccagagcaagtaagtcaggac

SEQ ID NO: 71 - DNA, 36 bp, synthetic construct
  misc_feature 1..36: note = source = /note="Description of Artificial Sequence: Synthetic oligonucleotide"
  source 1..36: mol_type = other DNA
gagaatcgaaatgctttaaacagtaaccagactccc

SEQ ID NO: 72 - DNA, 32 bp, synthetic construct
  misc_feature 1..32: note = source = /note="Description of Artificial Sequence: Synthetic oligonucleotide"
  source 1..32: mol_type = other DNA
cataaatctctttaccagtctggagcaaacaa

SEQ ID NO: 73 - DNA, 31 bp, synthetic construct
  misc_feature 1..31: note = source = /note="Description of Artificial Sequence: Synthetic oligonucleotide"
  source 1..31: mol_type = other DNA
ccaaaatagcgagatcaggaaaaaggctttt

SEQ ID NO: 74 - DNA, 28 bp, synthetic construct
  misc_feature 1..28: note = source = /note="Description of Artificial Sequence: Synthetic oligonucleotide"
  source 1..28: mol_type = other DNA
cctcgtaaaatgataaatataagaagtt

SEQ ID NO: 75 - DNA, 44 bp, synthetic construct
  misc_feature 1..44: note = source = /note="Description of Artificial Sequence: Synthetic oligonucleotide"
  source 1..44: mol_type = other DNA
gcaatcattgaacggaagcaaactccaatcaaagcgtcagaaaa

SEQ ID NO: 76 - DNA, 21 bp, synthetic construct
  misc_feature 1..21: note = source = /note="Description of Artificial Sequence: Synthetic oligonucleotide"
  source 1..21: mol_type = other DNA
gggtaataatgaacggaaaaa

SEQ ID NO: 77 - DNA, 28 bp, synthetic construct
  misc_feature 1..28: note = source = /note="Description of Artificial Sequence: Synthetic oligonucleotide"
  source 1..28: mol_type = other DNA
ggagagcctcaggaccctgtaaagaatt

SEQ ID NO: 78 - DNA, 32 bp, synthetic construct
  misc_feature 1..32: note = source = /note="Description of Artificial Sequence: Synthetic oligonucleotide"
  source 1..32: mol_type = other DNA
atgtaagcctttatttcaactattacagtgcg

SEQ ID NO: 79 - DNA, 44 bp, synthetic construct
  misc_feature 1..44: note = source = /note="Description of Artificial Sequence: Synthetic oligonucleotide"
  source 1..44: mol_type = other DNA
atgcaatactttgtagaaagattcatcaaacaacatgcaaggat

SEQ ID NO: 80 - DNA, 28 bp, synthetic construct
  misc_feature 1..28: note = source = /note="Description of Artificial Sequence: Synthetic oligonucleotide"
  source 1..28: mol_type = other DNA
agcaataagtaggtaaggcaaggcaatg

SEQ ID NO: 81 - DNA, 36 bp, synthetic construct
  misc_feature 1..36: note = source = /note="Description of Artificial Sequence: Synthetic oligonucleotide"
  source 1..36: mol_type = other DNA
agaacccttaatcattcttgagatggtttaatagta

SEQ ID NO: 82 - DNA, 24 bp, synthetic construct
  misc_feature 1..24: note = source = /note="Description of Artificial Sequence: Synthetic oligonucleotide"
  source 1..24: mol_type = other DNA
cctgttcaacttcatatattttaa

SEQ ID NO: 83 - DNA, 21 bp, synthetic construct
  misc_feature 1..21: note = source = /note="Description of Artificial Sequence: Synthetic oligonucleotide"
  source 1..21: mol_type = other DNA
tgattccctgatattcatatg

SEQ ID NO: 84 - DNA, 32 bp, synthetic construct
  misc_feature 1..32: note = source = /note="Description of Artificial Sequence: Synthetic oligonucleotide"
  source 1..32: mol_type = other DNA
tggcatcatagtagtagagacagtcaaatcac

SEQ ID NO: 85 - DNA, 44 bp, synthetic construct
  misc_feature 1..44: note = source = /note="Description of Artificial Sequence: Synthetic oligonucleotide"
  source 1..44: mol_type = other DNA
aagtgtcaataattgtaccaaaaacattcataaagcgggcgcga

SEQ ID NO: 86 - DNA, 31 bp, synthetic construct
  misc_feature 1..31: note = source = /note="Description of Artificial Sequence: Synthetic oligonucleotide"
  source 1..31: mol_type = other DNA
caactaaagtacggactaaattcttgtctgg

SEQ ID NO: 87 - DNA, 28 bp, synthetic construct
  misc_feature 1..28: note = source = /note="Description of Artificial Sequence: Synthetic oligonucleotide"
  source 1..28: mol_type = other DNA
tttaaattctgccgcaaatgttcattcc

SEQ ID NO: 88 - DNA, 36 bp, synthetic construct
  misc_feature 1..36: note = source = /note="Description of Artificial Sequence: Synthetic oligonucleotide"
  source 1..36: mol_type = other DNA
catcaatagctatattttcatttgtaaatcggcctg

SEQ ID NO: 89 - DNA, 36 bp, synthetic construct
  misc_feature 1..36: note = source = /note="Description of Artificial Sequence: Synthetic oligonucleotide"
  source 1..36: mol_type = other DNA
tttttgagattgctccttttgataatttagtttacc

SEQ ID NO: 90 - DNA, 28 bp, synthetic construct
  misc_feature 1..28: note = source = /note="Description of Artificial Sequence: Synthetic oligonucleotide"
  source 1..28: mol_type = other DNA
tttaaattcgagggtcaggaccgaaaga

SEQ ID NO: 91 - DNA, 31 bp, synthetic construct
  misc_feature 1..31: note = source = /note="Description of Artificial Sequence: Synthetic oligonucleotide"
  source 1..31: mol_type = other DNA
tgcatcaaaaagataattgagctttaagagg

SEQ ID NO: 92 - DNA, 44 bp, synthetic construct
  misc_feature 1..44: note = source = /note="Description of Artificial Sequence: Synthetic oligonucleotide"
  source 1..44: mol_type = other DNA
aagcttagagagtgaccattagatacatacgagtagagaggtca

SEQ ID NO: 93 - DNA, 32 bp, synthetic construct
  misc_feature 1..32: note = source = /note="Description of Artificial Sequence: Synthetic oligonucleotide"
  source 1..32: mol_type = other DNA
tggcttagctgaatatccggagagggtagcta

SEQ ID NO: 94 - DNA, 21 bp, synthetic construct
  misc_feature 1..21: note = source = /note="Description of Artificial Sequence: Synthetic oligonucleotide"
  source 1..21: mol_type = other DNA
cgcgttttagatctaccggat

SEQ ID NO: 95 - DNA, 21 bp, synthetic construct
  misc_feature 1..21: note = source = /note="Description of Artificial Sequence: Synthetic oligonucleotide"
  source 1..21: mol_type = other DNA
accagaacgagtagtaaattg

SEQ ID NO: 96 - DNA, 21 bp, synthetic construct
  misc_feature 1..21: note = source = /note="Description of Artificial Sequence: Synthetic oligonucleotide"
  source 1..21: mol_type = other DNA
gactattatagtcagaagcaa

SEQ ID NO: 97 - DNA, 21 bp, synthetic construct
  misc_feature 1..21: note = source = /note="Description of Artificial Sequence: Synthetic oligonucleotide"
  source 1..21: mol_type = other DNA
atcgtaaaactagcatgtcaa

SEQ ID NO: 98 - DNA, 21 bp, synthetic construct
  misc_feature 1..21: note = source = /note="Description of Artificial Sequence: Synthetic oligonucleotide"
  source 1..21: mol_type = other DNA
tgctgtagctcaacatgtttt

SEQ ID NO: 99 - DNA, 21 bp, synthetic construct
  misc_feature 1..21: note = source = /note="Description of Artificial Sequence: Synthetic oligonucleotide"
  source 1..21: mol_type = other DNA
gaattaccttatgcgatttta

SEQ ID NO: 100 - DNA, 21 bp, synthetic construct
  misc_feature 1..21: note = source = /note="Description of Artificial Sequence: Synthetic oligonucleotide"
  source 1..21: mol_type = other DNA
accctcgtttaccagacgacg

SEQ ID NO: 101 - DNA, 21 bp, synthetic construct
  misc_feature 1..21: note = source = /note="Description of Artificial Sequence: Synthetic oligonucleotide"
  source 1..21: mol_type = other DNA
attcaaaagggtgagaaaggc

SEQ ID NO: 102 - DNA, 21 bp, synthetic construct
  misc_feature 1..21: note = source = /note="Description of Artificial Sequence: Synthetic oligonucleotide"
  source 1..21: mol_type = other DNA
attaacatccaataaatcata

SEQ ID NO: 103 - DNA, 21 bp, synthetic construct
  misc_feature 1..21: note = source = /note="Description of Artificial Sequence: Synthetic oligonucleotide"
  source 1..21: mol_type = other DNA
aggctatcaggtcattgcctg

SEQ ID NO: 104 - DNA, 21 bp, synthetic construct
  misc_feature 1..21: note = source = /note="Description of Artificial Sequence: Synthetic oligonucleotide"
  source 1..21: mol_type = other DNA
ccgttctagctgataaattaa

SEQ ID NO: 105 - DNA, 32 bp, synthetic construct
  misc_feature 1..32: note = source = /note="Description of Artificial Sequence: Synthetic oligonucleotide"
  source 1..32: mol_type = other DNA
aatagcgacaaaagaaccgtgcatctaacttt

SEQ ID NO: 106 - DNA, 36 bp, synthetic construct
  misc_feature 1..36: note = source = /note="Description of Artificial Sequence: Synthetic oligonucleotide"
  source 1..36: mol_type = other DNA
aatcattgtaaccctcgtttaccattaaacagcact

SEQ ID NO: 107 - DNA, 44 bp, synthetic construct
  misc_feature 1..44: note = source = /note="Description of Artificial Sequence: Synthetic oligonucleotide"
  source 1..44: mol_type = other DNA
aaagaagagcaattcagaaaacgagaatcaaatgctgacgacga

SEQ ID NO: 108 - DNA, 28 bp, synthetic construct
  misc_feature 1..28: note = source = /note="Description of Artificial Sequence: Synthetic oligonucleotide"
  source 1..28: mol_type = other DNA
atcacacattcaggcatagtattcatca

SEQ ID NO: 109 - DNA, 21 bp, synthetic construct
  misc_feature 1..21: note = source = /note="Description of Artificial Sequence: Synthetic oligonucleotide"
  source 1..21: mol_type = other DNA
aggaatactgaattactaacg

SEQ ID NO: 110 - DNA, 31 bp, synthetic construct
  misc_feature 1..31: note = source = /note="Description of Artificial Sequence: Synthetic oligonucleotide"
  source 1..31: mol_type = other DNA
gaacaacattattattttggaggccaggtag

SEQ ID NO: 111 - DNA, 21 bp, synthetic construct
  misc_feature 1..21: note = source = /note="Description of Artificial Sequence: Synthetic oligonucleotide"
  source 1..21: mol_type = other DNA
tattcattgtcacgtttaaaa

SEQ ID NO: 112 - DNA, 36 bp, synthetic construct
  misc_feature 1..36: note = source = /note="Description of Artificial Sequence: Synthetic oligonucleotide"
  source 1..36: mol_type = other DNA
tgggatagaccctgactattatagctccttttaggt

SEQ ID NO: 113 - DNA, 44 bp, synthetic construct
  misc_feature 1..44: note = source = /note="Description of Artificial Sequence: Synthetic oligonucleotide"
  source 1..44: mol_type = other DNA
aatacaaaaatcgataagaggtcatttttttaattgtcagaagc

SEQ ID NO: 114 - DNA, 28 bp, synthetic construct
  misc_feature 1..28: note = source = /note="Description of Artificial Sequence: Synthetic oligonucleotide"
  source 1..28: mol_type = other DNA
ctttgaatccccccataaatctgcggaa

SEQ ID NO: 115 - DNA, 32 bp, synthetic construct
  misc_feature 1..32: note = source = /note="Description of Artificial Sequence: Synthetic oligonucleotide"
  source 1..32: mol_type = other DNA
gcatcaaaggaagcccggcggattgaccgtaa

SEQ ID NO: 116 - DNA, 31 bp, synthetic construct
  misc_feature 1..31: note = source = /note="Description of Artificial Sequence: Synthetic oligonucleotide"
  source 1..31: mol_type = other DNA
tgtttagactggattaagaaagatagcgtcc

SEQ ID NO: 117 - DNA, 44 bp, synthetic construct
  misc_feature 1..44: note = source = /note="Description of Artificial Sequence: Synthetic oligonucleotide"
  source 1..44: mol_type = other DNA
ttatatgcaatgttgcctgagagtctggaaaggctatcaaaagg

SEQ ID NO: 118 - DNA, 32 bp, synthetic construct
  misc_feature 1..32: note = source = /note="Description of Artificial Sequence: Synthetic oligonucleotide"
  source 1..32: mol_type = other DNA
ccggagacccatcaattattttgttaaaattc

SEQ ID NO: 119 - DNA, 28 bp, synthetic construct
  misc_feature 1..28: note = source = /note="Description of Artificial Sequence: Synthetic oligonucleotide"
  source 1..28: mol_type = other DNA
agtagcctttattattttaagaccctgt

SEQ ID NO: 120 - DNA, 31 bp, synthetic construct
  misc_feature 1..31: note = source = /note="Description of Artificial Sequence: Synthetic oligonucleotide"
  source 1..31: mol_type = other DNA
aaatcggttgtaccaatcaagtcaaaaaaca

SEQ ID NO: 121 - DNA, 21 bp, synthetic construct
  misc_feature 1..21: note = source = /note="Description of Artificial Sequence: Synthetic oligonucleotide"
  source 1..21: mol_type = other DNA
cgggagaatttttgttaagct

SEQ ID NO: 122 - DNA, 36 bp, synthetic construct
  misc_feature 1..36: note = source = /note="Description of Artificial Sequence: Synthetic oligonucleotide"
  source 1..36: mol_type = other DNA
gcattaaaatgtgtaggtaaagattcaggtcacctg

SEQ ID NO: 123 - DNA, 24 bp, synthetic construct
  misc_feature 1..24: note = source = /note="Description of Artificial Sequence: Synthetic oligonucleotide"
  source 1..24: mol_type = other DNA
caggggacgttgaatcagaaaagc

SEQ ID NO: 124 - DNA, 36 bp, synthetic construct
  misc_feature 1..36: note = source = /note="Description of Artificial Sequence: Synthetic oligonucleotide"
  source 1..36: mol_type = other DNA
cggttgatggaagaaactcattataccagtcaaaga

SEQ ID NO: 125 - DNA, 44 bp, synthetic construct
  misc_feature 1..44: note = source = /note="Description of Artificial Sequence: Synthetic oligonucleotide"
  source 1..44: mol_type = other DNA
cccaagaatcgacgccaaaaggaattactaatgcagtgtcaatc

SEQ ID NO: 126 - DNA, 28 bp, synthetic construct
  misc_feature 1..28: note = source = /note="Description of Artificial Sequence: Synthetic oligonucleotide"
  source 1..28: mol_type = other DNA
tatttttgataagcaagataaattaaaa

SEQ ID NO: 127 - DNA, 28 bp, synthetic construct
  misc_feature 1..28: note = source = /note="Description of Artificial Sequence: Synthetic oligonucleotide"
  source 1..28: mol_type = other DNA
cggtagagatctcaaacaagaatgccgg

SEQ ID NO: 128 - DNA, 32 bp, synthetic construct
  misc_feature 1..32: note = source = /note="Description of Artificial Sequence: Synthetic oligonucleotide"
  source 1..32: mol_type = other DNA
ttgtaatcgtaaaactagcaatacataatgaa

SEQ ID NO: 129 - DNA, 28 bp, synthetic construct
  misc_feature 1..28: note = source = /note="Description of Artificial Sequence: Synthetic oligonucleotide"
  source 1..28: mol_type = other DNA
actaggtcaatagaaaaggtttgaccat

SEQ ID NO: 130 - DNA, 32 bp, synthetic construct
  misc_feature 1..32: note = source = /note="Description of Artificial Sequence: Synthetic oligonucleotide"
  source 1..32: mol_type = other DNA
ggcaaggccaaaattaataggaacgccatcaa

SEQ ID NO: 131 - DNA, 36 bp, synthetic construct
  misc_feature 1..36: note = source = /note="Description of Artificial Sequence: Synthetic oligonucleotide"
  source 1..36: mol_type = other DNA
aaataattatagtagtagcattaaggataaaattct

SEQ ID NO: 132 - DNA, 31 bp, synthetic construct
  misc_feature 1..31: note = source = /note="Description of Artificial Sequence: Synthetic oligonucleotide"
  source 1..31: mol_type = other DNA
aattctgcgaacgaattagaaagagtagatt

SEQ ID NO: 133 - DNA, 21 bp, synthetic construct
  misc_feature 1..21: note = source = /note="Description of Artificial Sequence: Synthetic oligonucleotide"
  source 1..21: mol_type = other DNA
tcgcaaatcgcgtctgttccc

SEQ ID NO: 134 - DNA, 44 bp, synthetic construct
  misc_feature 1..44: note = source = /note="Description of Artificial Sequence: Synthetic oligonucleotide"
  source 1..44: mol_type = other DNA
tagtggcatcaaatttttagaaccctcacaacgcaacatccaat

SEQ ID NO: 135 - DNA, 28 bp, synthetic construct
  misc_feature 1..28: note = source = /note="Description of Artificial Sequence: Synthetic oligonucleotide"
  source 1..28: mol_type = other DNA
tgctagagagtacggatggcgaagcaaa

SEQ ID NO: 136 - DNA, 44 bp, synthetic construct
  misc_feature 1..44: note = source = /note="Description of Artificial Sequence: Synthetic oligonucleotide"
  source 1..44: mol_type = other DNA
accgttagagcttcatttggggcgcgagctgtttagctcaacat

SEQ ID NO: 137 - DNA, 21 bp, synthetic construct
  misc_feature 1..21: note = source = /note="Description of Artificial Sequence: Synthetic oligonucleotide"
  source 1..21: mol_type = other DNA
tcaggattcaacccgtttaat

SEQ ID NO: 138 - DNA, 31 bp, synthetic construct
  misc_feature 1..31: note = source = /note="Description of Artificial Sequence: Synthetic oligonucleotide"
  source 1..31: mol_type = other DNA
tcgagcttcaaagccggtgaagtagaaccag

SEQ ID NO: 139 - DNA, 32 bp, synthetic construct
  misc_feature 1..32: note = source = /note="Description of Artificial Sequence: Synthetic oligonucleotide"
  source 1..32: mol_type = other DNA
tgcaactatctggaagtcaacattaaatgtga

SEQ ID NO: 140 - DNA, 36 bp, synthetic construct
  misc_feature 1..36: note = source = /note="Description of Artificial Sequence: Synthetic oligonucleotide"
  source 1..36: mol_type = other DNA
gcgagtaagaatataatgctgtagctatattttaat

SEQ ID NO: 141 - DNA, 18 bp, synthetic construct
  misc_feature 1..18: note = source = /note="Description of Artificial Sequence: Synthetic oligonucleotide"
  source 1..18: mol_type = other DNA
atttaaattgtaaacgtt

SEQ ID NO: 142 - DNA, 18 bp, synthetic construct
  misc_feature 1..18: note = source = /note="Description of Artificial Sequence: Synthetic oligonucleotide"
  source 1..18: mol_type = other DNA
tcattccatataacagtt

SEQ ID NO: 143 - DNA, 18 bp, synthetic construct
  misc_feature 1..18: note = source = /note="Description of Artificial Sequence: Synthetic oligonucleotide"
  source 1..18: mol_type = other DNA
tgtagatgggcgcatcgt

SEQ ID NO: 144 - DNA, 18 bp, synthetic construct
  misc_feature 1..18: note = source = /note="Description of Artificial Sequence: Synthetic oligonucleotide"
  source 1..18: mol_type = other DNA
gattctccgtgggaacaa

SEQ ID NO: 145 - DNA, 18 bp, synthetic construct
  misc_feature 1..18: note = source = /note="Description of Artificial Sequence: Synthetic oligonucleotide"
  source 1..18: mol_type = other DNA
atcagctcattttttaac

SEQ ID NO: 146 - DNA, 18 bp, synthetic construct
  misc_feature 1..18: note = source = /note="Description of Artificial Sequence: Synthetic oligonucleotide"
  source 1..18: mol_type = other DNA
cttcctgtagccagcttt

SEQ ID NO: 147 - DNA, 18 bp, synthetic construct
  misc_feature 1..18: note = source = /note="Description of Artificial Sequence: Synthetic oligonucleotide"
  source 1..18: mol_type = other DNA
tctacgttaataaaacga

SEQ ID NO: 148 - DNA, 18 bp, synthetic construct
  misc_feature 1..18: note = source = /note="Description of Artificial Sequence: Synthetic oligonucleotide"
  source 1..18: mol_type = other DNA
tttgccagagggggtaat

SEQ ID NO: 149 - DNA, 18 bp, synthetic construct
  misc_feature 1..18: note = source = /note="Description of Artificial Sequence: Synthetic oligonucleotide"
  source 1..18: mol_type = other DNA
aagacttcaaatatcgcg

SEQ ID NO: 150 - DNA, 18 bp, synthetic construct
  misc_feature 1..18: note = source = /note="Description of Artificial Sequence: Synthetic oligonucleotide"
  source 1..18: mol_type = other DNA
tatgcgattttaagaact

SEQ ID NO: 151 - DNA, 18 bp, synthetic construct
  misc_feature 1..18: note = source = /note="Description of Artificial Sequence: Synthetic oligonucleotide"
  source 1..18: mol_type = other DNA
caataaagcctcagagca

SEQ ID NO: 152 - DNA, 18 bp, synthetic construct
  misc_feature 1..18: note = source = /note="Description of Artificial Sequence: Synthetic oligonucleotide"
  source 1..18: mol_type = other DNA
gatattcaaccgttctag

SEQ ID NO: 153 - DNA, 29 bp, synthetic construct
  misc_feature 1..29: note = source = /note="Description of Artificial Sequence: Synthetic oligonucleotide"
  source 1..29: mol_type = other DNA
gtagtcgtcgcttagcttagtgagaagag

SEQ ID NO: 154 - DNA, 38 bp, synthetic construct
  misc_feature 1..38: note = source = /note="Description of Artificial Sequence: Synthetic oligonucleotide"
  source 1..38: mol_type = other DNA
cgttagaacgcgaaaaaagcctgtgctaagttcattag

SEQ ID NO: 155 - DNA, 40 bp, synthetic construct
  misc_feature 1..40: note = source = /note="Description of Artificial Sequence: Synthetic oligonucleotide"
  source 1..40: mol_type = other DNA
aatagtttaacgtcagatgaaccatatcatcattttatat

SEQ ID NO: 156 - DNA, 25 bp, synthetic construct
  misc_feature 1..25: note = source = /note="Description of Artificial Sequence: Synthetic oligonucleotide"
  source 1..25: mol_type = other DNA
attatttgcacgtaaaacagattgc

SEQ ID NO: 157 - DNA, 43 bp, synthetic construct
  misc_feature 1..43: note = source = /note="Description of Artificial Sequence: Synthetic oligonucleotide"
  source 1..43: mol_type = other DNA
aataaatgatgaaacaaattaattacatttaacaatttcactt

SEQ ID NO: 158 - DNA, 32 bp, synthetic construct
  misc_feature 1..32: note = source = /note="Description of Artificial Sequence: Synthetic oligonucleotide"
  source 1..32: mol_type = other DNA
cttgaaaacgaattatagcaaaagaagagaaa

SEQ ID NO: 159 - DNA, 32 bp, synthetic construct
  misc_feature 1..32: note = source = /note="Description of Artificial Sequence: Synthetic oligonucleotide"
  source 1..32: mol_type = other DNA
cgacaataaacaacattgcagaactgttttga

SEQ ID NO: 160 - DNA, 29 bp, synthetic construct
  misc_feature 1..29: note = source = /note="Description of Artificial Sequence: Synthetic oligonucleotide"
  source 1..29: mol_type = other DNA
aaaagaaaattttcagccaagttacaaaa

SEQ ID NO: 161 - DNA, 45 bp, synthetic construct
  misc_feature 1..45: note = source = /note="Description of Artificial Sequence: Synthetic oligonucleotide"
  source 1..45: mol_type = other DNA
gaacaattactaggaaaactttttcaaataatagattcaattcga

SEQ ID NO: 162 - DNA, 35 bp, synthetic construct
  misc_feature 1..35: note = source = /note="Description of Artificial Sequence: Synthetic oligonucleotide"
  source 1..35: mol_type = other DNA
gagcggaattgtttggggaagggttagaacctata

SEQ ID NO: 163 - DNA, 32 bp, synthetic construct
  misc_feature 1..32: note = source = /note="Description of Artificial Sequence: Synthetic oligonucleotide"
  source 1..32: mol_type = other DNA
aaccattaaatctaacagtaacaataacggat

SEQ ID NO: 164 - DNA, 22 bp, synthetic construct
  misc_feature 1..22: note = source = /note="Description of Artificial Sequence: Synthetic oligonucleotide"
  source 1..22: mol_type = other DNA
tcgcgcagaggcatagcgaatt

SEQ ID NO: 165 - DNA, 23 bp, synthetic construct
  misc_feature 1..23: note = source = /note="Description of Artificial Sequence: Synthetic oligonucleotide"
  source 1..23: mol_type = other DNA
tcgcctgattgctattagacgaa

SEQ ID NO: 166 - DNA, 34 bp, synthetic construct
  misc_feature 1..34: note = source = /note="Description of Artificial Sequence: Synthetic oligonucleotide"
  source 1..34: mol_type = other DNA
gcaattcatcaatataatcctgattatcatcgcg

SEQ ID NO: 167 - DNA, 37 bp, synthetic construct
  misc_feature 1..37: note = source = /note="Description of Artificial Sequence: Synthetic oligonucleotide"
  source 1..37: mol_type = other DNA
ataatacattaagacaaattaattttgtaacattaaa

SEQ ID NO: 168 - DNA, 20 bp, synthetic construct
  misc_feature 1..20: note = source = /note="Description of Artificial Sequence: Synthetic oligonucleotide"
  source 1..20: mol_type = other DNA
attaaggatttagaagtttg

SEQ ID NO: 169 - DNA, 35 bp, synthetic construct
  misc_feature 1..35: note = source = /note="Description of Artificial Sequence: Synthetic oligonucleotide"
  source 1..35: mol_type = other DNA
tacagctttgccctttacaaaagagccgtcaatag

SEQ ID NO: 170 - DNA, 28 bp, synthetic construct
  misc_feature 1..28: note = source = /note="Description of Artificial Sequence: Synthetic oligonucleotide"
  source 1..28: mol_type = other DNA
tgagtgaataacatggaaacccagacga

SEQ ID NO: 171 - DNA, 35 bp, synthetic construct
  misc_feature 1..35: note = source = /note="Description of Artificial Sequence: Synthetic oligonucleotide"
  source 1..35: mol_type = other DNA
tcaatagtgaatttactatatgtattttccctatg

SEQ ID NO: 172 - DNA, 45 bp, synthetic construct
  misc_feature 1..45: note = source = /note="Description of Artificial Sequence: Synthetic oligonucleotide"
  source 1..45: mol_type = other DNA
aattaaaatgctgttatcaacaatagataaattctgtagtacata

SEQ ID NO: 173 - DNA, 45 bp, synthetic construct
  misc_feature 1..45: note = source = /note="Description of Artificial Sequence: Synthetic oligonucleotide"
  source 1..45: mol_type = other DNA
ttttacttgcttcgcgcctgtatgcaaatccaatcgctgagacgc

SEQ ID NO: 174 - DNA, 29 bp, synthetic construct
  misc_feature 1..29: note = source = /note="Description of Artificial Sequence: Synthetic oligonucleotide"
  source 1..29: mol_type = other DNA
tcctgattatcagatattactttgagatg

SEQ ID NO: 175 - DNA, 35 bp, synthetic construct
  misc_feature 1..35: note = source = /note="Description of Artificial Sequence: Synthetic oligonucleotide"
  source 1..35: mol_type = other DNA
tatcatatgcgttatacaaattcgaatcataaaga

SEQ ID NO: 176 - DNA, 22 bp, synthetic construct
  misc_feature 1..22: note = source = /note="Description of Artificial Sequence: Synthetic oligonucleotide"
  source 1..22: mol_type = other DNA
ttgctcacttgcctatcattcc

SEQ ID NO: 177 - DNA, 32 bp, synthetic construct
  misc_feature 1..32: note = source = /note="Description of Artificial Sequence: Synthetic oligonucleotide"
  source 1..32: mol_type = other DNA
tagagtctgtcccttctgacacgtggcacaga

SEQ ID NO: 178 - DNA, 29 bp, synthetic construct
  misc_feature 1..29: note = source = /note="Description of Artificial Sequence: Synthetic oligonucleotide"
  source 1..29: mol_type = other DNA
aataaaaccctcaattgaggaaaatatct

SEQ ID NO: 179 - DNA, 35 bp, synthetic construct
  misc_feature 1..35: note = source = /note="Description of Artificial Sequence: Synthetic oligonucleotide"
  source 1..35: mol_type = other DNA
ttaggagcactaacattttagtttctggtcaaaag

SEQ ID NO: 180 - DNA, 32 bp, synthetic construct
  misc_feature 1..32: note = source = /note="Description of Artificial Sequence: Synthetic oligonucleotide"
  source 1..32: mol_type = other DNA
tttacccgccagccattgcaacaggaaaagcc

SEQ ID NO: 181 - DNA, 40 bp, synthetic construct
  misc_feature 1..40: note = source = /note="Description of Artificial Sequence: Synthetic oligonucleotide"
  source 1..40: mol_type = other DNA
atataatcgccatatttaacaacgccaatctttccttgag

SEQ ID NO: 182 - DNA, 28 bp, synthetic construct
  misc_feature 1..28: note = source = /note="Description of Artificial Sequence: Synthetic oligonucleotide"
  source 1..28: mol_type = other DNA
catcaccttgctgagccagcaagccaac

SEQ ID NO: 183 - DNA, 20 bp, synthetic construct
  misc_feature 1..20: note = source = /note="Description of Artificial Sequence: Synthetic oligonucleotide"
  source 1..20: mol_type = other DNA
aaggtctgagaagtgtatgg

SEQ ID NO: 184 - DNA, 48 bp, synthetic construct
  misc_feature 1..48: note = source = /note="Description of Artificial Sequence: Synthetic oligonucleotide"
  source 1..48: mol_type = other DNA
aatggtttgaaacgggtattaaaccaagtaccaaagggatgccaccga

SEQ ID NO: 185 - DNA, 45 bp, synthetic construct
  misc_feature 1..45: note = source = /note="Description of Artificial Sequence: Synthetic oligonucleotide"
  source 1..45: mol_type = other DNA
caataaatttcattaaataagaataaacaccagtataagcaaatg

SEQ ID NO: 186 - DNA, 32 bp, synthetic construct
  misc_feature 1..32: note = source = /note="Description of Artificial Sequence: Synthetic oligonucleotide"
  source 1..32: mol_type = other DNA
gctcaacagtagggctgtgataaaaatttctt

SEQ ID NO: 187 - DNA, 23 bp, synthetic construct
  misc_feature 1..23: note = source = /note="Description of Artificial Sequence: Synthetic oligonucleotide"
  source 1..23: mol_type = other DNA
caatatttttgattttataaaat

SEQ ID NO: 188 - DNA, 45 bp, synthetic construct
  misc_feature 1..45: note = source = /note="Description of Artificial Sequence: Synthetic oligonucleotide"
  source 1..45: mol_type = other DNA
gctgagaacctcataaggcgtcttctgacctaaatttatctatct

SEQ ID NO: 189 - DNA, 29 bp, synthetic construct
  misc_feature 1..29: note = source = /note="Description of Artificial Sequence: Synthetic oligonucleotide"
  source 1..29: mol_type = other DNA
aagaataccgaccgttaattgagccagaa

SEQ ID NO: 190 - DNA, 43 bp, synthetic construct
  misc_feature 1..43: note = source = /note="Description of Artificial Sequence: Synthetic oligonucleotide"
  source 1..43: mol_type = other DNA
gtcacccaccagcagaaggcggtcagtattaacaccgcctcac

SEQ ID NO: 191 - DNA, 32 bp, synthetic construct
  misc_feature 1..32: note = source = /note="Description of Artificial Sequence: Synthetic oligonucleotide"
  source 1..32: mol_type = other DNA
atcaacaggccctaaataccgaacgaaacgac

SEQ ID NO: 192 - DNA, 29 bp, synthetic construct
  misc_feature 1..29: note = source = /note="Description of Artificial Sequence: Synthetic oligonucleotide"
  source 1..29: mol_type = other DNA
aatacatcaaagggactagtctttaatgc

SEQ ID NO: 193 - DNA, 38 bp, synthetic construct
  misc_feature 1..38: note = source = /note="Description of Artificial Sequence: Synthetic oligonucleotide"
  source 1..38: mol_type = other DNA
caataagtgcgcaacttaattggcagattcaccacagt

SEQ ID NO: 194 - DNA, 24 bp, synthetic construct
  misc_feature 1..24: note = source = /note="Description of Artificial Sequence: Synthetic oligonucleotide"
  source 1..24: mol_type = other DNA
taaccgttgtagctgattagttta

SEQ ID NO: 195 - DNA, 22 bp, synthetic construct
  misc_feature 1..22: note = source = /note="Description of Artificial Sequence: Synthetic oligonucleotide"
  source 1..22: mol_type = other DNA
gcgaactgatattgaaaggaat

SEQ ID NO: 196 - DNA, 40 bp, synthetic construct
  misc_feature 1..40: note = source = /note="Description of Artificial Sequence: Synthetic oligonucleotide"
  source 1..40: mol_type = other DNA
ctatattctggccaacagaggaaatggaaataacatggta

SEQ ID NO: 197 - DNA, 45 bp, synthetic construct
  misc_feature 1..45: note = source = /note="Description of Artificial Sequence: Synthetic oligonucleotide"
  source 1..45: mol_type = other DNA
gaaccatcacgcatcagtgagtttagacaggaacggtacgccaga

SEQ ID NO: 198 - DNA, 35 bp, synthetic construct
  misc_feature 1..35: note = source = /note="Description of Artificial Sequence: Synthetic oligonucleotide"
  source 1..35: mol_type = other DNA
aactatcgacgctcattgacgctcaatcgtctata

SEQ ID NO: 199 - DNA, 29 bp, synthetic construct
  misc_feature 1..29: note = source = /note="Description of Artificial Sequence: Synthetic oligonucleotide"
  source 1..29: mol_type = other DNA
tgaacgctacagctttcctcgagcgggag

SEQ ID NO: 200 - DNA, 43 bp, synthetic construct
  misc_feature 1..43: note = source = /note="Description of Artificial Sequence: Synthetic oligonucleotide"
  source 1..43: mol_type = other DNA
tttaaggggaaagccgaggaagggaagaaagcgaaaggagggc

SEQ ID NO: 201 - DNA, 28 bp, synthetic construct
  misc_feature 1..28: note = source = /note="Description of Artificial Sequence: Synthetic oligonucleotide"
  source 1..28: mol_type = other DNA
caccacacccgccaagtgtaggcagagg

SEQ ID NO: 202 - DNA, 47 bp, synthetic construct
  misc_feature 1..47: note = source = /note="Description of Artificial Sequence: Synthetic oligonucleotide"
  source 1..47: mol_type = other DNA
gtataacagggagaattaactgaacacccaagttttcaatagcacgc

SEQ ID NO: 203 - DNA, 20 bp, synthetic construct
  misc_feature 1..20: note = source = /note="Description of Artificial Sequence: Synthetic oligonucleotide"
  source 1..20: mol_type = other DNA
gttagattaccgcgccttgg

SEQ ID NO: 204 - DNA, 32 bp, synthetic construct
  misc_feature 1..32: note = source = /note="Description of Artificial Sequence: Synthetic oligonucleotide"
  source 1..32: mol_type = other DNA
gagcacgtgaaccctatagagcttgaccgtca

SEQ ID NO: 205 - DNA, 32 bp, synthetic construct
  misc_feature 1..32: note = source = /note="Description of Artificial Sequence: Synthetic oligonucleotide"
  source 1..32: mol_type = other DNA
cattttcgagccagtattacgagcatgtcccg

SEQ ID NO: 206 - DNA, 48 bp, synthetic construct
  misc_feature 1..48: note = source = /note="Description of Artificial Sequence: Synthetic oligonucleotide"
  source 1..48: mol_type = other DNA
atttatataaagtaccgacaaaaggtaacctgaacaaaatcaagtccg

SEQ ID NO: 207 - DNA, 44 bp, synthetic construct
  misc_feature 1..44: note = source = /note="Description of Artificial Sequence: Synthetic oligonucleotide"
  source 1..44: mol_type = other DNA
ctattttggagcctaataaacagccatattataacataaatcta

SEQ ID NO: 208 - DNA, 40 bp, synthetic construct
  misc_feature 1..40: note = source = /note="Description of Artificial Sequence: Synthetic oligonucleotide"
  source 1..40: mol_type = other DNA
ggtccctttacagagagaatttatcccagaggtttttaca

SEQ ID NO: 209 - DNA, 22 bp, synthetic construct
  misc_feature 1..22: note = source = /note="Description of Artificial Sequence: Synthetic oligonucleotide"
  source 1..22: mol_type = other DNA
gcactaaatcgataacgtgggc

SEQ ID NO: 210 - DNA, 37 bp, synthetic construct
  misc_feature 1..37: note = source = /note="Description of Artificial Sequence: Synthetic oligonucleotide"
  source 1..37: mol_type = other DNA
cctccggaatatcccatcctaatataagagatatcct

SEQ ID NO: 211 - DNA, 29 bp, synthetic construct
  misc_feature 1..29: note = source = /note="Description of Artificial Sequence: Synthetic oligonucleotide"
  source 1..29: mol_type = other DNA
gaacccgcaatagcaggaggtgccgtaaa

SEQ ID NO: 212 - DNA, 45 bp, synthetic construct
  misc_feature 1..45: note = source = /note="Description of Artificial Sequence: Synthetic oligonucleotide"
  source 1..45: mol_type = other DNA
gcgtagaacaagcaccaatcaataatcgggtaatttagcggtcac

SEQ ID NO: 213 - DNA, 45 bp, synthetic construct
  misc_feature 1..45: note = source = /note="Description of Artificial Sequence: Synthetic oligonucleotide"
  source 1..45: mol_type = other DNA
agaaagcaaatcggtctgagagactaccttttaggacatcgttaa

SEQ ID NO: 214 - DNA, 40 bp, synthetic construct
  misc_feature 1..40: note = source = /note="Description of Artificial Sequence: Synthetic oligonucleotide"
  source 1..40: mol_type = other DNA
caaatccaacgctaacgagcgtctttccacacccagcgaa

SEQ ID NO: 215 - DNA, 38 bp, synthetic construct
  misc_feature 1..38: note = source = /note="Description of Artificial Sequence: Synthetic oligonucleotide"
  source 1..38: mol_type = other DNA
gaatcgctagcgggcttaaagaaacgattttttgaaaa

SEQ ID NO: 216 - DNA, 24 bp, synthetic construct
  misc_feature 1..24: note = source = /note="Description of Artificial Sequence: Synthetic oligonucleotide"
  source 1..24: mol_type = other DNA
gaggcgttttagcacttgcggatc

SEQ ID NO: 217 - DNA, 45 bp, synthetic construct
  misc_feature 1..45: note = source = /note="Description of Artificial Sequence: Synthetic oligonucleotide"
  source 1..45: mol_type = other DNA
gccttagaaaaatcttaggttgggttataaaatcataagatatag

SEQ ID NO: 218 - DNA, 45 bp, synthetic construct
  misc_feature 1..45: note = source = /note="Description of Artificial Sequence: Synthetic oligonucleotide"
  source 1..45: mol_type = other DNA
gctggcgcgcttaatgtagaaaagccgtttttattttatcaatca

SEQ ID NO: 219 - DNA, 35 bp, synthetic construct
  misc_feature 1..35: note = source = /note="Description of Artificial Sequence: Synthetic oligonucleotide"
  source 1..35: mol_type = other DNA
ctaaacaggaggccgctcatcgactatggtttaac

SEQ ID NO: 220 - DNA, 44 bp, synthetic construct
  misc_feature 1..44: note = source = /note="Description of Artificial Sequence: Synthetic oligonucleotide"
  source 1..44: mol_type = other DNA
tagtaccaaaatagcgagaggcttttgccataacgctcataaat

SEQ ID NO: 221 - DNA, 20 bp, synthetic construct
  misc_feature 1..20: note = source = /note="Description of Artificial Sequence: Synthetic oligonucleotide"
  source 1..20: mol_type = other DNA
cgagtagtaagattcatcag

SEQ ID NO: 222 - DNA, 28 bp, synthetic construct
  misc_feature 1..28: note = source = /note="Description of Artificial Sequence: Synthetic oligonucleotide"
  source 1..28: mol_type = other DNA
atagcgtcaccataaaggtaacgccagg

SEQ ID NO: 223 - DNA, 24 bp, synthetic construct
  misc_feature 1..24: note = source = /note="Description of Artificial Sequence: Synthetic oligonucleotide"
  source 1..24: mol_type = other DNA
gttttcccagtccagaggggcgga

SEQ ID NO: 224 - DNA, 41 bp, synthetic construct
  misc_feature 1..41: note = source = /note="Description of Artificial Sequence: Synthetic oligonucleotide"
  source 1..41: mol_type = other DNA
gataaaaaggaagatttagaagttttgcacgacgtttaaac

SEQ ID NO: 225 - DNA, 32 bp, synthetic construct
  misc_feature 1..32: note = source = /note="Description of Artificial Sequence: Synthetic oligonucleotide"
  source 1..32: mol_type = other DNA
attcattgaaatgcttgtaaaacgacgcagaa

SEQ ID NO: 226 - DNA, 44 bp, synthetic construct
  misc_feature 1..44: note = source = /note="Description of Artificial Sequence: Synthetic oligonucleotide"
  source 1..44: mol_type = other DNA
atcgcaaaaggaattacgaggcatagtacgataaaaaaaatgtt

SEQ ID NO: 227 - DNA, 44 bp, synthetic construct
  misc_feature 1..44: note = source = /note="Description of Artificial Sequence: Synthetic oligonucleotide"
  source 1..44: mol_type = other DNA
ttgataccacataatattttgttaaaatttaaattgcggaacaa

SEQ ID NO: 228 - DNA, 31 bp, synthetic construct
  misc_feature 1..31: note = source = /note="Description of Artificial Sequence: Synthetic oligonucleotide"
  source 1..31: mol_type = other DNA
agttcagaaaacgagaatgcaatactggtaa

SEQ ID NO: 229 - DNA, 24 bp, synthetic construct
  misc_feature 1..24: note = source = /note="Description of Artificial Sequence: Synthetic oligonucleotide"
  source 1..24: mol_type = other DNA
ctaataaacgtttcaactaatgca

SEQ ID NO: 230 - DNA, 31 bp, synthetic construct
  misc_feature 1..31: note = source = /note="Description of Artificial Sequence: Synthetic oligonucleotide"
  source 1..31: mol_type = other DNA
aatctacgttaataccctcaatccaaacgaa

SEQ ID NO: 231 - DNA, 21 bp, synthetic construct
  misc_feature 1..21: note = source = /note="Description of Artificial Sequence: Synthetic oligonucleotide"
  source 1..21: mol_type = other DNA
caggtagaaaattgggagaaa

SEQ ID NO: 232 - DNA, 20 bp, synthetic construct
  misc_feature 1..20: note = source = /note="Description of Artificial Sequence: Synthetic oligonucleotide"
  source 1..20: mol_type = other DNA
acgccagccttcaaatatcg

SEQ ID NO: 233 - DNA, 41 bp, synthetic construct
  misc_feature 1..41: note = source = /note="Description of Artificial Sequence: Synthetic oligonucleotide"
  source 1..41: mol_type = other DNA
gaagctgtcgagttaattagctcaacatatcggtgcgacca

SEQ ID NO: 234 - DNA, 44 bp, synthetic construct
  misc_feature 1..44: note = source = /note="Description of Artificial Sequence: Synthetic oligonucleotide"
  source 1..44: mol_type = other DNA
cgttcttcaaagccctcgtttaccagacagcaacacaagattaa

SEQ ID NO: 235 - DNA, 31 bp, synthetic construct
  misc_feature 1..31: note = source = /note="Description of Artificial Sequence: Synthetic oligonucleotide"
  source 1..31: mol_type = other DNA
cagaagcaaagcgggtagaaacgaattgcat

SEQ ID NO: 236 - DNA, 44 bp, synthetic construct
  misc_feature 1..44: note = source = /note="Description of Artificial Sequence: Synthetic oligonucleotide"
  source 1..44: mol_type = other DNA
caacagagcttaattgctgaatataatgcaaactccgattccca

SEQ ID NO: 237 - DNA, 24 bp, synthetic construct
  misc_feature 1..24: note = source = /note="Description of Artificial Sequence: Synthetic oligonucleotide"
  source 1..24: mol_type = other DNA
caaatatcataacgaaccagaccg

SEQ ID NO: 238 - DNA, 44 bp, synthetic construct
  misc_feature 1..44: note = source = /note="Description of Artificial Sequence: Synthetic oligonucleotide"
  source 1..44: mol_type = other DNA
agttaacaggtcaggattagagagtaccgatggctttaaagtac

SEQ ID NO: 239 - DNA, 28 bp, synthetic construct
  misc_feature 1..28: note = source = /note="Description of Artificial Sequence: Synthetic oligonucleotide"
  source 1..28: mol_type = other DNA
gaagtttcggtcaatagctgcgcaactg

SEQ ID NO: 240 - DNA, 31 bp, synthetic construct
  misc_feature 1..31: note = source = /note="Description of Artificial Sequence: Synthetic oligonucleotide"
  source 1..31: mol_type = other DNA
ttagatacatttcgcaaatattccatatatg

SEQ ID NO: 241 - DNA, 24 bp, synthetic construct
  misc_feature 1..24: note = source = /note="Description of Artificial Sequence: Synthetic oligonucleotide"
  source 1..24: mol_type = other DNA
ttgggaagggcggttttaaataac

SEQ ID NO: 242 - DNA, 21 bp, synthetic construct
  misc_feature 1..21: note = source = /note="Description of Artificial Sequence: Synthetic oligonucleotide"
  source 1..21: mol_type = other DNA
ccgaaagatggcgaaaatagt

SEQ ID NO: 243 - DNA, 32 bp, synthetic construct
  misc_feature 1..32: note = source = /note="Description of Artificial Sequence: Synthetic oligonucleotide"
  source 1..32: mol_type = other DNA
attctgcgtttagtttgggcctcttcgctatt

SEQ ID NO: 244 - DNA, 28 bp, synthetic construct
  misc_feature 1..28: note = source = /note="Description of Artificial Sequence: Synthetic oligonucleotide"
  source 1..28: mol_type = other DNA
cagctcatttttgccccaaaatgtgagc

SEQ ID NO: 245 - DNA, 24 bp, synthetic construct
  misc_feature 1..24: note = source = /note="Description of Artificial Sequence: Synthetic oligonucleotide"
  source 1..24: mol_type = other DNA
tcaaaacattaaaacaggaagatt

SEQ ID NO: 246 - DNA, 20 bp, synthetic construct
  misc_feature 1..20: note = source = /note="Description of Artificial Sequence: Synthetic oligonucleotide"
  source 1..20: mol_type = other DNA
gatgggcgtcgtaaaactag

SEQ ID NO: 247 - DNA, 28 bp, synthetic construct
  misc_feature 1..28: note = source = /note="Description of Artificial Sequence: Synthetic oligonucleotide"
  source 1..28: mol_type = other DNA
gtataagcaaatgcattaaacgcgtctg

SEQ ID NO: 248 - DNA, 24 bp, synthetic construct
  misc_feature 1..24: note = source = /note="Description of Artificial Sequence: Synthetic oligonucleotide"
  source 1..24: mol_type = other DNA
agcacctgtaatgttgataatcag

SEQ ID NO: 249 - DNA, 21 bp, synthetic construct
  misc_feature 1..21: note = source = /note="Description of Artificial Sequence: Synthetic oligonucleotide"
  source 1..21: mol_type = other DNA
aacggtaacatcgtaatatca

SEQ ID NO: 250 - DNA, 32 bp, synthetic construct
  misc_feature 1..32: note = source = /note="Description of Artificial Sequence: Synthetic oligonucleotide"
  source 1..32: mol_type = other DNA
gagtaacattctccgtaggtcacgttggtgta

SEQ ID NO: 251 - DNA, 44 bp, synthetic construct
  misc_feature 1..44: note = source = /note="Description of Artificial Sequence: Synthetic oligonucleotide"
  source 1..44: mol_type = other DNA
gtagccagtaagaactattgtgaattaccttattgaccgtgcca

SEQ ID NO: 252 - DNA, 44 bp, synthetic construct
  misc_feature 1..44: note = source = /note="Description of Artificial Sequence: Synthetic oligonucleotide"
  source 1..44: mol_type = other DNA
catggtaccccgacttttgcgggagaagattatgacaacaagag

SEQ ID NO: 253 - DNA, 40 bp, synthetic construct
  misc_feature 1..40: note = source = /note="Description of Artificial Sequence: Synthetic oligonucleotide"
  source 1..40: mol_type = other DNA
aaaataacatattcaatccaataggaacaatgggatggga

SEQ ID NO: 254 - DNA, 24 bp, synthetic construct
  misc_feature 1..24: note = source = /note="Description of Artificial Sequence: Synthetic oligonucleotide"
  source 1..24: mol_type = other DNA
acaaacggcggatgcgatttcttt

SEQ ID NO: 255 - DNA, 24 bp, synthetic construct
  misc_feature 1..24: note = source = /note="Description of Artificial Sequence: Synthetic oligonucleotide"
  source 1..24: mol_type = other DNA
catcaaataatttttttgttaaat

SEQ ID NO: 256 - DNA, 31 bp, synthetic construct
  misc_feature 1..31: note = source = /note="Description of Artificial Sequence: Synthetic oligonucleotide"
  source 1..31: mol_type = other DNA
ggtcattgcctgagtcggaacccgagtctgg

SEQ ID NO: 257 - DNA, 44 bp, synthetic construct
  misc_feature 1..44: note = source = /note="Description of Artificial Sequence: Synthetic oligonucleotide"
  source 1..44: mol_type = other DNA
gccgaaagctaaatcggttgtaccaaaatttatttcgtaatgtg

SEQ ID NO: 258 - DNA, 44 bp, synthetic construct
  misc_feature 1..44: note = source = /note="Description of Artificial Sequence: Synthetic oligonucleotide"
  source 1..44: mol_type = other DNA
gcaatagcaaaataagaggtcatttttgtaattgctaatagtag

SEQ ID NO: 259 - DNA, 44 bp, synthetic construct
  misc_feature 1..44: note = source = /note="Description of Artificial Sequence: Synthetic oligonucleotide"
  source 1..44: mol_type = other DNA
ctgaaacgcaaggataaaaatttttagacagagcatgagacagt

SEQ ID NO: 260 - DNA, 31 bp, synthetic construct
  misc_feature 1..31: note = source = /note="Description of Artificial Sequence: Synthetic oligonucleotide"
  source 1..31: mol_type = other DNA
ctagctgataaattaatgcagggtgagatgc

SEQ ID NO: 261 - DNA, 24 bp, synthetic construct
  misc_feature 1..24: note = source = /note="Description of Artificial Sequence: Synthetic oligonucleotide"
  source 1..24: mol_type = other DNA
tactccttttgattaagcaataaa

SEQ ID NO: 262 - DNA, 41 bp, synthetic construct
  misc_feature 1..41: note = source = /note="Description of Artificial Sequence: Synthetic oligonucleotide"
  source 1..41: mol_type = other DNA
gcctaccagaatggcaactcatatatttgcactccaccgtt

SEQ ID NO: 263 - DNA, 21 bp, synthetic construct
  misc_feature 1..21: note = source = /note="Description of Artificial Sequence: Synthetic oligonucleotide"
  source 1..21: mol_type = other DNA
acatccaagtgccggagcgag

SEQ ID NO: 264 - DNA, 20 bp, synthetic construct
  misc_feature 1..20: note = source = /note="Description of Artificial Sequence: Synthetic oligonucleotide"
  source 1..20: mol_type = other DNA
cgcttctgtaaatcatacag

SEQ ID NO: 265 - DNA, 24 bp, synthetic construct
  misc_feature 1..24: note = source = /note="Description of Artificial Sequence: Synthetic oligonucleotide"
  source 1..24: mol_type = other DNA
ctcaggaagatctaaatgcaaaag

SEQ ID NO: 266 - DNA, 28 bp, synthetic construct
  misc_feature 1..28: note = source = /note="Description of Artificial Sequence: Synthetic oligonucleotide"
  source 1..28: mol_type = other DNA
gattcaaacggagagggacagtatcggc

SEQ ID NO: 267 - DNA, 32 bp, synthetic construct
  misc_feature 1..32: note = source = /note="Description of Artificial Sequence: Synthetic oligonucleotide"
  source 1..32: mol_type = other DNA
caaatcacatattcaagccagctttccggcac

SEQ ID NO: 268 - DNA, 31 bp, synthetic construct
  misc_feature 1..31: note = source = /note="Description of Artificial Sequence: Synthetic oligonucleotide"
  source 1..31: mol_type = other DNA
ctgaaaaggtggcaatatgcatcatcaattc

SEQ ID NO: 269 - DNA, 25 bp, synthetic construct
  misc_feature 1..25: note = source = /note="Description of Artificial Sequence: Synthetic oligonucleotide"
  source 1..25: mol_type = other DNA
aaaaatcaggtctttaccctgacta

SEQ ID NO: 270 - DNA, 25 bp, synthetic construct
  misc_feature 1..25: note = source = /note="Description of Artificial Sequence: Synthetic oligonucleotide"
  source 1..25: mol_type = other DNA
ctgtttagctatattttcatttggg

SEQ ID NO: 271 - DNA, 25 bp, synthetic construct
  misc_feature 1..25: note = source = /note="Description of Artificial Sequence: Synthetic oligonucleotide"
  source 1..25: mol_type = other DNA
tgagatggtttaatttcaactttaa

SEQ ID NO: 272 - DNA, 25 bp, synthetic construct
  misc_feature 1..25: note = source = /note="Description of Artificial Sequence: Synthetic oligonucleotide"
  source 1..25: mol_type = other DNA
ccaggcaaagcgccattcgccattc

SEQ ID NO: 273 - DNA, 25 bp, synthetic construct
  misc_feature 1..25: note = source = /note="Description of Artificial Sequence: Synthetic oligonucleotide"
  source 1..25: mol_type = other DNA
ctcattataccagtcaggacgttgg

SEQ ID NO: 274 - DNA, 25 bp, synthetic construct
  misc_feature 1..25: note = source = /note="Description of Artificial Sequence: Synthetic oligonucleotide"
  source 1..25: mol_type = other DNA
gtgcatctgccagtttgaggggacg

SEQ ID NO: 275 - DNA, 25 bp, synthetic construct
  misc_feature 1..25: note = source = /note="Description of Artificial Sequence: Synthetic oligonucleotide"
  source 1..25: mol_type = other DNA
agctatttttgagagatctacaaag

SEQ ID NO: 276 - DNA, 25 bp, synthetic construct
  misc_feature 1..25: note = source = /note="Description of Artificial Sequence: Synthetic oligonucleotide"
  source 1..25: mol_type = other DNA
gggatgtgctgcaaggcgattaagt

SEQ ID NO: 277 - DNA, 32 bp, synthetic construct
  misc_feature 1..32: note = source = /note="Description of Artificial Sequence: Synthetic oligonucleotide"
  source 1..32: mol_type = other DNA
aaacaggtcgtcaaactatcaaaacattacca

SEQ ID NO: 278 - DNA, 32 bp, synthetic construct
  misc_feature 1..32: note = source = /note="Description of Artificial Sequence: Synthetic oligonucleotide"
  source 1..32: mol_type = other DNA
tcctaagcgccgaagaagctggagtgtctgta

SEQ ID NO: 279 - DNA, 33 bp, synthetic construct
  misc_feature 1..33: note = source = /note="Description of Artificial Sequence: Synthetic oligonucleotide"
  source 1..33: mol_type = other DNA
gcgcacctaagaaaaagagtaaacagattaaac

SEQ ID NO: 280 - DNA, 33 bp, synthetic construct
  misc_feature 1..33: note = source = /note="Description of Artificial Sequence: Synthetic oligonucleotide"
  source 1..33: mol_type = other DNA
gacgggagtggagaatgtagctttaacagaagt

SEQ ID NO: 281 - DNA, 32 bp, synthetic construct
  misc_feature 1..32: note = source = /note="Description of Artificial Sequence: Synthetic oligonucleotide"
  source 1..32: mol_type = other DNA
gagaaccagcttatcagaaaaaaagtagtgtt

SEQ ID NO: 282 - DNA, 32 bp, synthetic construct
  misc_feature 1..32: note = source = /note="Description of Artificial Sequence: Synthetic oligonucleotide"
  source 1..32: mol_type = other DNA
aacagtcggggttaatcgtgccaagattttat

SEQ ID NO: 283 - DNA, 32 bp, synthetic construct
  misc_feature 1..32: note = source = /note="Description of Artificial Sequence: Synthetic oligonucleotide"
  source 1..32: mol_type = other DNA
ctcaggaagtcgcagtaggcggaagtactgaa

SEQ ID NO: 284 - DNA, 32 bp, synthetic construct
  misc_feature 1..32: note = source = /note="Description of Artificial Sequence: Synthetic oligonucleotide"
  source 1..32: mol_type = other DNA
tggcgagaaataaaagtctgaaacgtgccatg

SEQ ID NO: 285 - DNA, 33 bp, synthetic construct
  misc_feature 1..33: note = source = /note="Description of Artificial Sequence: Synthetic oligonucleotide"
  source 1..33: mol_type = other DNA
catgaaacgcaaaggtcaatataacttgaatta

SEQ ID NO: 286 - DNA, 40 bp, synthetic construct
  misc_feature 1..40: note = source = /note="Description of Artificial Sequence: Synthetic oligonucleotide"
  source 1..40: mol_type = other DNA
gcattaaccgttgacagatgtatcacaacctgaattaaaa

SEQ ID NO: 287 - DNA, 40 bp, synthetic construct
  misc_feature 1..40: note = source = /note="Description of Artificial Sequence: Synthetic oligonucleotide"
  source 1..40: mol_type = other DNA
tctctttagccagcaatatcggtacatacaaaccagaaaa

SEQ ID NO: 288 - DNA, 40 bp, synthetic construct
  misc_feature 1..40: note = source = /note="Description of Artificial Sequence: Synthetic oligonucleotide"
  source 1..40: mol_type = other DNA
tggtatcagttgttatagatattcacggcgcttgttcacc

SEQ ID NO: 289 - DNA, 60 bp, synthetic construct
  misc_feature 1..60: note = source = /note="Description of Artificial Sequence: Synthetic oligonucleotide"
  source 1..60: mol_type = other DNA
cagcaatcaagaaggtatacgaagggcgaataagtacgcgtggttcatcttcacactact

SEQ ID NO: 290 - DNA, 56 bp, synthetic construct
  misc_feature 1..56: note = source = /note="Description of Artificial Sequence: Synthetic oligonucleotide"
  source 1..56: mol_type = other DNA
gacggaaggagtgagagcgccaacggcgcatgaactgtaatcatcttcacactact

SEQ ID NO: 291 - DNA, 40 bp, synthetic construct
  misc_feature 1..40: note = source = /note="Description of Artificial Sequence: Synthetic oligonucleotide"
  source 1..40: mol_type = other DNA
tgggaagccttcaatttaacatcatcatcttcacactact

SEQ ID NO: 292 - DNA, 40 bp, synthetic construct
  misc_feature 1..40: note = source = /note="Description of Artificial Sequence: Synthetic oligonucleotide"
  source 1..40: mol_type = other DNA
tggtatagccagatgcccagcagttcatcttcacactact

SEQ ID NO: 293 - DNA, 48 bp, synthetic construct
  misc_feature 1..48: note = source = /note="Description of Artificial Sequence: Synthetic oligonucleotide"
  source 1..48: mol_type = other DNA
cggtacggcaacggaatcttgattgcggagcatcatcttcacactact

SEQ ID NO: 294 - DNA, 56 bp, synthetic construct
  misc_feature 1..56: note = source = /note="Description of Artificial Sequence: Synthetic oligonucleotide"
  source 1..56: mol_type = other DNA
cacgtccaaatggtcagcgtcataagagggtctgtttcaatcatcttcacactact

SEQ ID NO: 295 - DNA, 44 bp, synthetic construct
  misc_feature 1..44: note = source = /note="Description of Artificial Sequence: Synthetic oligonucleotide"
  source 1..44: mol_type = other DNA
tatatcaggcatataaccctaagctcattcatcttcacactact

SEQ ID NO: 296 - DNA, 44 bp, synthetic construct
  misc_feature 1..44: note = source = /note="Description of Artificial Sequence: Synthetic oligonucleotide"
  source 1..44: mol_type = other DNA
gtcaggcaagttagtcaaagaacatccttcatcttcacactact

SEQ ID NO: 297 - DNA, 60 bp, synthetic construct
  misc_feature 1..60: note = source = /note="Description of Artificial Sequence: Synthetic oligonucleotide"
  source 1..60: mol_type = other DNA
accgaagacatcatggaaacatcagaccagcaaggaagccataatcatcttcacactact

SEQ ID NO: 298 - DNA, 40 bp, synthetic construct
  misc_feature 1..40: note = source = /note="Description of Artificial Sequence: Synthetic oligonucleotide"
  source 1..40: mol_type = other DNA
ggtagaagtcgtgttttaccttcttcatcttcacactact

SEQ ID NO: 299 - DNA, 60 bp, synthetic construct
  misc_feature 1..60: note = source = /note="Description of Artificial Sequence: Synthetic oligonucleotide"
  source 1..60: mol_type = other DNA
gatggcagcatttggcaggaggaacagtatgcaaattagctaattcatcttcacactact

SEQ ID NO: 300 - DNA, 48 bp, synthetic construct
  misc_feature 1..48: note = source = /note="Description of Artificial Sequence: Synthetic oligonucleotide"
  source 1..48: mol_type = other DNA
cacgcggcttaaacttgaacggaagcgcataatcatcttcacactact

SEQ ID NO: 301 - DNA, 48 bp, synthetic construct
  misc_feature 1..48: note = source = /note="Description of Artificial Sequence: Synthetic oligonucleotide"
  source 1..48: mol_type = other DNA
atatcagcagactcaaaaaaaatttaggcagttcatcttcacactact

SEQ ID NO: 302 - DNA, 40 bp, synthetic construct
  misc_feature 1..40: note = source = /note="Description of Artificial Sequence: Synthetic oligonucleotide"
  source 1..40: mol_type = other DNA
atctgtctacagtagagtcacatatcatcttcacactact

SEQ ID NO: 303 - DNA, 56 bp, synthetic construct
  misc_feature 1..56: note = source = /note="Description of Artificial Sequence: Synthetic oligonucleotide"
  source 1..56: mol_type = other DNA
catactgactatataaacggtcacattaacccctcacacttcatcttcacactact

SEQ ID NO: 304 - DNA, 40 bp, synthetic construct
  misc_feature 1..40: note = source = /note="Description of Artificial Sequence: Synthetic oligonucleotide"
  source 1..40: mol_type = other DNA
agtttgaaccactccatctcaccatcatcttcacactact

SEQ ID NO: 305 - DNA, 44 bp, synthetic construct
  misc_feature 1..44: note = source = /note="Description of Artificial Sequence: Synthetic oligonucleotide"
  source 1..44: mol_type = other DNA
ccttaccaacagtctgaatgtagggtcgtcatcttcacactact

SEQ ID NO: 306 - DNA, 40 bp, synthetic construct
  misc_feature 1..40: note = source = /note="Description of Artificial Sequence: Synthetic oligonucleotide"
  source 1..40: mol_type = other DNA
tggtcttttatcacgaagtctaactcatcttcacactact

SEQ ID NO: 307 - DNA, 40 bp, synthetic construct
  misc_feature 1..40: note = source = /note="Description of Artificial Sequence: Synthetic oligonucleotide"
  source 1..40: mol_type = other DNA
cctacgagtcagtccttccagaacacgaacagggtctgtt

SEQ ID NO: 308 - DNA, 44 bp, synthetic construct
  misc_feature 1..44: note = source = /note="Description of Artificial Sequence: Synthetic oligonucleotide"
  source 1..44: mol_type = other DNA
cctacgagtcagtcctgcatgaagataagcaggtcaatagatgt

SEQ ID NO: 309 - DNA, 40 bp, synthetic construct
  misc_feature 1..40: note = source = /note="Description of Artificial Sequence: Synthetic oligonucleotide"
  source 1..40: mol_type = other DNA
cctacgagtcagtcctctcagcggcaaaattagcggcttg

SEQ ID NO: 310 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctaaatcaataacaggcattgaagaaaacctaacgtt

SEQ ID NO: 311 - DNA, 48 bp, synthetic construct
  misc_feature 1..48: note = source = /note="Description of Artificial Sequence: Synthetic oligonucleotide"
  source 1..48: mol_type = other DNA
cctacgagtcagtcctatgagggaatagcaaggaaagacgcgccagcg

SEQ ID NO: 312 - DNA, 48 bp, synthetic construct
  misc_feature 1..48: note = source = /note="Description of Artificial Sequence: Synthetic oligonucleotide"
  source 1..48: mol_type = other DNA
cctacgagtcagtcctcgtttggtagattagaggacggttaagaaata

SEQ ID NO: 313 - DNA, 44 bp, synthetic construct
  misc_feature 1..44: note = source = /note="Description of Artificial Sequence: Synthetic oligonucleotide"
  source 1..44: mol_type = other DNA
cctacgagtcagtcctaaatggtaaagatgggacgctcggcgcc

SEQ ID NO: 314 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctcaatcaccttcgccgcgctttagcgttacgaacaa

SEQ ID NO: 315 - DNA, 40 bp, synthetic construct
  misc_feature 1..40: note = source = /note="Description of Artificial Sequence: Synthetic oligonucleotide"
  source 1..40: mol_type = other DNA
cctacgagtcagtcctaaaataatccacttaaaataccat

SEQ ID NO: 316 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctcatcgaacagaacggcaacaaatgcttaaaagcgg

SEQ ID NO: 317 - DNA, 48 bp, synthetic construct
  misc_feature 1..48: note = source = /note="Description of Artificial Sequence: Synthetic oligonucleotide"
  source 1..48: mol_type = other DNA
cctacgagtcagtcctcggcgttaatgattgaagattgcgtccactgc

SEQ ID NO: 318 - DNA, 44 bp, synthetic construct
  misc_feature 1..44: note = source = /note="Description of Artificial Sequence: Synthetic oligonucleotide"
  source 1..44: mol_type = other DNA
cctacgagtcagtcctggaaacacttcttgcattcctgaatgaa

SEQ ID NO: 319 - DNA, 32 bp, synthetic construct
  misc_feature 1..32: note = source = /note="Description of Artificial Sequence: Synthetic oligonucleotide"
  source 1..32: mol_type = other DNA
acagtattgttatcggtagcaagcaacttaat

SEQ ID NO: 320 - DNA, 32 bp, synthetic construct
  misc_feature 1..32: note = source = /note="Description of Artificial Sequence: Synthetic oligonucleotide"
  source 1..32: mol_type = other DNA
ccactgttttaacagtcgggagagaatataac

SEQ ID NO: 321 - DNA, 33 bp, synthetic construct
  misc_feature 1..33: note = source = /note="Description of Artificial Sequence: Synthetic oligonucleotide"
  source 1..33: mol_type = other DNA
caggtaaaccaccagttaggaacatccatgcct

SEQ ID NO: 322 - DNA, 33 bp, synthetic construct
  misc_feature 1..33: note = source = /note="Description of Artificial Sequence: Synthetic oligonucleotide"
  source 1..33: mol_type = other DNA
taacaggtctttataccatccttcaatcacctt

SEQ ID NO: 323 - DNA, 32 bp, synthetic construct
  misc_feature 1..32: note = source = /note="Description of Artificial Sequence: Synthetic oligonucleotide"
  source 1..32: mol_type = other DNA
acagaatgcaagcgcaagagtaaagtaggcgg

SEQ ID NO: 324 - DNA, 32 bp, synthetic construct
  misc_feature 1..32: note = source = /note="Description of Artificial Sequence: Synthetic oligonucleotide"
  source 1..32: mol_type = other DNA
gaatgccaccggaggcggctttttaacgcggc

SEQ ID NO: 325 - DNA, 33 bp, synthetic construct
  misc_feature 1..33: note = source = /note="Description of Artificial Sequence: Synthetic oligonucleotide"
  source 1..33: mol_type = other DNA
agaacagcaaccgcataaaagtctgcaagagca

SEQ ID NO: 326 - DNA, 32 bp, synthetic construct
  misc_feature 1..32: note = source = /note="Description of Artificial Sequence: Synthetic oligonucleotide"
  source 1..32: mol_type = other DNA
aatggcaggaaacaaaactagggatttagcca

SEQ ID NO: 327 - DNA, 32 bp, synthetic construct
  misc_feature 1..32: note = source = /note="Description of Artificial Sequence: Synthetic oligonucleotide"
  source 1..32: mol_type = other DNA
gaagcaatatttaataccagcatcgagccttg

SEQ ID NO: 328 - DNA, 33 bp, synthetic construct
  misc_feature 1..33: note = source = /note="Description of Artificial Sequence: Synthetic oligonucleotide"
  source 1..33: mol_type = other DNA
atgcgaaaaaagcatcaggaacaaaccgcctcc

SEQ ID NO: 329 - DNA, 32 bp, synthetic construct
  misc_feature 1..32: note = source = /note="Description of Artificial Sequence: Synthetic oligonucleotide"
  source 1..32: mol_type = other DNA
aaactcctcagaaaaaaagtttgagtgagaac

SEQ ID NO: 330 - DNA, 32 bp, synthetic construct
  misc_feature 1..32: note = source = /note="Description of Artificial Sequence: Synthetic oligonucleotide"
  source 1..32: mol_type = other DNA
aaacaatttagacatggcgccaccacatgatt

SEQ ID NO: 331 - DNA, 44 bp, synthetic construct
  misc_feature 1..44: note = source = /note="Description of Artificial Sequence: Synthetic oligonucleotide"
  source 1..44: mol_type = other DNA
catatcaagcaaaccttcaaggcagacttcatcttcacactact

SEQ ID NO: 332 - DNA, 60 bp, synthetic construct
  misc_feature 1..60: note = source = /note="Description of Artificial Sequence: Synthetic oligonucleotide"
  source 1..60: mol_type = other DNA
aaatccggtcggcaatagttgcatgaccagcaaggaagccataatcatcttcacactact

SEQ ID NO: 333 - DNA, 48 bp, synthetic construct
  misc_feature 1..48: note = source = /note="Description of Artificial Sequence: Synthetic oligonucleotide"
  source 1..48: mol_type = other DNA
caaccaaacgtcaaccttatcagctttagtaatcatcttcacactact

SEQ ID NO: 334 - DNA, 56 bp, synthetic construct
  misc_feature 1..56: note = source = /note="Description of Artificial Sequence: Synthetic oligonucleotide"
  source 1..56: mol_type = other DNA
gacggaaggagtcatcatctcaacggcgaaaagtaagaaatcatcttcacactact

SEQ ID NO: 335 - DNA, 40 bp, synthetic construct
  misc_feature 1..40: note = source = /note="Description of Artificial Sequence: Synthetic oligonucleotide"
  source 1..40: mol_type = other DNA
agtttgtgcatctccatctcaccatcatcttcacactact

SEQ ID NO: 336 - DNA, 40 bp, synthetic construct
  misc_feature 1..40: note = source = /note="Description of Artificial Sequence: Synthetic oligonucleotide"
  source 1..40: mol_type = other DNA
atctgagagcgctgattaaggcaatcatcttcacactact

SEQ ID NO: 337 - DNA, 44 bp, synthetic construct
  misc_feature 1..44: note = source = /note="Description of Artificial Sequence: Synthetic oligonucleotide"
  source 1..44: mol_type = other DNA
agaaccctgaaacgtgccaaaaaatagttcatcttcacactact

SEQ ID NO: 338 - DNA, 40 bp, synthetic construct
  misc_feature 1..40: note = source = /note="Description of Artificial Sequence: Synthetic oligonucleotide"
  source 1..40: mol_type = other DNA
aacggagcgcattaaacttcagattcatcttcacactact

SEQ ID NO: 339 - DNA, 56 bp, synthetic construct
  misc_feature 1..56: note = source = /note="Description of Artificial Sequence: Synthetic oligonucleotide"
  source 1..56: mol_type = other DNA
atcattgtcagcagcaatctgacaagtaaactggccagagtcatcttcacactact

SEQ ID NO: 340 - DNA, 60 bp, synthetic construct
  misc_feature 1..60: note = source = /note="Description of Artificial Sequence: Synthetic oligonucleotide"
  source 1..60: mol_type = other DNA
aagcggagatgtcaatgtcatttgctgcatgaagtaatcataactcatcttcacactact

SEQ ID NO: 341 - DNA, 40 bp, synthetic construct
  misc_feature 1..40: note = source = /note="Description of Artificial Sequence: Synthetic oligonucleotide"
  source 1..40: mol_type = other DNA
tgcagacccataaaggacggtggttcatcttcacactact

SEQ ID NO: 342 - DNA, 48 bp, synthetic construct
  misc_feature 1..48: note = source = /note="Description of Artificial Sequence: Synthetic oligonucleotide"
  source 1..48: mol_type = other DNA
tcaaataacagtccaaggcgctttgcgagaaatcatcttcacactact

SEQ ID NO: 343 - DNA, 40 bp, synthetic construct
  misc_feature 1..40: note = source = /note="Description of Artificial Sequence: Synthetic oligonucleotide"
  source 1..40: mol_type = other DNA
aaggatccgcggagctcaggtgattcatcttcacactact

SEQ ID NO: 344 - DNA, 60 bp, synthetic construct
  misc_feature 1..60: note = source = /note="Description of Artificial Sequence: Synthetic oligonucleotide"
  source 1..60: mol_type = other DNA
aaacaggcaaacatcatagtcggaacgtcaatagtcacaccggctcatcttcacactact

SEQ ID NO: 345 - DNA, 56 bp, synthetic construct
  misc_feature 1..56: note = source = /note="Description of Artificial Sequence: Synthetic oligonucleotide"
  source 1..56: mol_type = other DNA
gtacagttagacaagcattacatttagtcacctcaccgaatcatcttcacactact

SEQ ID NO: 346 - DNA, 48 bp, synthetic construct
  misc_feature 1..48: note = source = /note="Description of Artificial Sequence: Synthetic oligonucleotide"
  source 1..48: mol_type = other DNA
actatcaaaaaaaattaaccaccaaccgaagatcatcttcacactact

SEQ ID NO: 347 - DNA, 40 bp, synthetic construct
  misc_feature 1..40: note = source = /note="Description of Artificial Sequence: Synthetic oligonucleotide"
  source 1..40: mol_type = other DNA
catccaaaggatagcggtaaggggtcatcttcacactact

SEQ ID NO: 348 - DNA, 44 bp, synthetic construct
  misc_feature 1..44: note = source = /note="Description of Artificial Sequence: Synthetic oligonucleotide"
  source 1..44: mol_type = other DNA
aaataatataacgccgaagattaccagctcatcttcacactact

SEQ ID NO: 349 - DNA, 48 bp, synthetic construct
  misc_feature 1..48: note = source = /note="Description of Artificial Sequence: Synthetic oligonucleotide"
  source 1..48: mol_type = other DNA
caaaacagagcaggagaaatttcatgaatgaatcatcttcacactact

SEQ ID NO: 350 - DNA, 40 bp, synthetic construct
  misc_feature 1..40: note = source = /note="Description of Artificial Sequence: Synthetic oligonucleotide"
  source 1..40: mol_type = other DNA
ggcgaataagtaagtcatgataactcatcttcacactact

SEQ ID NO: 351 - DNA, 60 bp, synthetic construct
  misc_feature 1..60: note = source = /note="Description of Artificial Sequence: Synthetic oligonucleotide"
  source 1..60: mol_type = other DNA
aggtgatacgcgttctgcggttccctattccactgcaacaattatcatcttcacactact

SEQ ID NO: 352 - DNA, 44 bp, synthetic construct
  misc_feature 1..44: note = source = /note="Description of Artificial Sequence: Synthetic oligonucleotide"
  source 1..44: mol_type = other DNA
cttgggtcgccaaggtactgcgcggcggtcatcttcacactact

SEQ ID NO: 353 - DNA, 40 bp, synthetic construct
  misc_feature 1..40: note = source = /note="Description of Artificial Sequence: Synthetic oligonucleotide"
  source 1..40: mol_type = other DNA
ctgacaatctttgattagcggttatcatcttcacactact

SEQ ID NO: 354 - DNA, 56 bp, synthetic construct
  misc_feature 1..56: note = source = /note="Description of Artificial Sequence: Synthetic oligonucleotide"
  source 1..56: mol_type = other DNA
aattttgaatcgaacaaccttatcacgagtcaacccctcatcatcttcacactact

SEQ ID NO: 355 - DNA, 48 bp, synthetic construct
  misc_feature 1..48: note = source = /note="Description of Artificial Sequence: Synthetic oligonucleotide"
  source 1..48: mol_type = other DNA
cctacgagtcagtcctgccacgacctcattagataacgagcgccagcg

SEQ ID NO: 356 - DNA, 44 bp, synthetic construct; identical to SEQ ID NO: 313
  misc_feature 1..44: note = source = /note="Description of Artificial Sequence: Synthetic oligonucleotide"
  source 1..44: mol_type = other DNA

SEQ ID NO: 357 - DNA, 40 bp, synthetic construct
  misc_feature 1..40: note = source = /note="Description of Artificial Sequence: Synthetic oligonucleotide"
  source 1..40: mol_type = other DNA
cctacgagtcagtccttggaaaatgtctgggattttggga

SEQ ID NO: 358 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctgacggaagatgagtgacgtcctttaccagcctcat

SEQ ID NO: 359 - DNA, 44 bp, synthetic construct
  misc_feature 1..44: note = source = /note="Description of Artificial Sequence: Synthetic oligonucleotide"
  source 1..44: mol_type = other DNA
cctacgagtcagtccttgaagaaacgttcttggcataagcagct

SEQ ID NO: 360 - DNA, 40 bp, synthetic construct
  misc_feature 1..40: note = source = /note="Description of Artificial Sequence: Synthetic oligonucleotide"
  source 1..40: mol_type = other DNA
cctacgagtcagtcctgccctaacgacgtataagtcgacc

SEQ ID NO: 361 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctattagaaaacactgttgaagcggcatgggtggcat

SEQ ID NO: 362 - DNA, 48 bp, synthetic construct
  misc_feature 1..48: note = source = /note="Description of Artificial Sequence: Synthetic oligonucleotide"
  source 1..48: mol_type = other DNA
cctacgagtcagtccttcatagccttagacgatgaccctcgtcataag

SEQ ID NO: 363 - DNA, 40 bp, synthetic construct
  misc_feature 1..40: note = source = /note="Description of Artificial Sequence: Synthetic oligonucleotide"
  source 1..40: mol_type = other DNA
cctacgagtcagtccttcagttaagtggacggcagaacat

SEQ ID NO: 364 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctaccaagcaccaaatagctggagtaacagtatggcg

SEQ ID NO: 365 - DNA, 44 bp, synthetic construct
  misc_feature 1..44: note = source = /note="Description of Artificial Sequence: Synthetic oligonucleotide"
  source 1..44: mol_type = other DNA
cctacgagtcagtcctttattgccagtccttgcatcatggcgac

SEQ ID NO: 366 - DNA, 48 bp, synthetic construct
  misc_feature 1..48: note = source = /note="Description of Artificial Sequence: Synthetic oligonucleotide"
  source 1..48: mol_type = other DNA
cctacgagtcagtccttaataaccaaatgcagacgctccccaaaccat

SEQ ID NO: 367 - DNA, 48 bp, synthetic construct
  misc_feature 1..48: note = source = /note="Description of Artificial Sequence: Synthetic oligonucleotide"
  source 1..48: mol_type = other DNA
cctacgagtcagtcctgcttcggcgcgttgacccaacagacgagtggt

SEQ ID NO: 368 - DNA, 40 bp, synthetic construct
  misc_feature 1..40: note = source = /note="Description of Artificial Sequence: Synthetic oligonucleotide"
  source 1..40: mol_type = other DNA
cctacgagtcagtccttaacctcagcggtagctttattcg

SEQ ID NO: 369 - DNA, 51 bp, synthetic construct
  misc_feature 1..51: note = source = /note="Description of Artificial Sequence: Synthetic oligonucleotide"
  source 1..51: mol_type = other DNA
cctacgagtcagtcctcactaatcgcgccctacctctttagtcgtagtgcc

SEQ ID NO: 370 - DNA, 44 bp, synthetic construct
  misc_feature 1..44: note = source = /note="Description of Artificial Sequence: Synthetic oligonucleotide"
  source 1..44: mol_type = other DNA
cctacgagtcagtcctacggtcacactgaacgcataatcatggt

SEQ ID NO: 371 - DNA, 40 bp, synthetic construct
  misc_feature 1..40: note = source = /note="Description of Artificial Sequence: Synthetic oligonucleotide"
  source 1..40: mol_type = other DNA
tagcaccaagtgccagcctgcaactatcagaactggagac

SEQ ID NO: 372 - DNA, 40 bp, synthetic construct
  misc_feature 1..40: note = source = /note="Description of Artificial Sequence: Synthetic oligonucleotide"
  source 1..40: mol_type = other DNA
cagtagtgattggtatcagggttaaatgcttaacagtaga

SEQ ID NO: 373 - DNA, 40 bp, synthetic construct
  misc_feature 1..40: note = source = /note="Description of Artificial Sequence: Synthetic oligonucleotide"
  source 1..40: mol_type = other DNA
aaaacgaaaaagcacctttagcgtaatatcggtttggtca

SEQ ID NO: 374 - DNA, 40 bp, synthetic construct
  misc_feature 1..40: note = source = /note="Description of Artificial Sequence: Synthetic oligonucleotide"
  source 1..40: mol_type = other DNA
cagcttatggtgtctgtaaaacagtgacgatgcaaaaatt

SEQ ID NO: 375 - DNA, 41 bp, synthetic construct
  misc_feature 1..41: note = source = /note="Description of Artificial Sequence: Synthetic oligonucleotide"
  source 1..41: mol_type = other DNA
tagcaccaagtgccagcctgcaactagctcactcagtctca

SEQ ID NO: 376 - DNA, 16 bp, synthetic construct
  misc_feature 1..16: note = source = /note="Description of Artificial Sequence: Synthetic oligonucleotide"
  source 1..16: mol_type = other DNA
tatcagaactggagac

SEQ ID NO: 377 - DNA, 41 bp, synthetic construct
  misc_feature 1..41: note = source = /note="Description of Artificial Sequence: Synthetic oligonucleotide"
  source 1..41: mol_type = other DNA
cagtagtgattggtatcagggttatagctcactcagtctca

SEQ ID NO: 378 - DNA, 16 bp, synthetic construct
  misc_feature 1..16: note = source = /note="Description of Artificial Sequence: Synthetic oligonucleotide"
  source 1..16: mol_type = other DNA
aatgcttaacagtaga

SEQ ID NO: 379 - DNA, 41 bp, synthetic construct
  misc_feature 1..41: note = source = /note="Description of Artificial Sequence: Synthetic oligonucleotide"
  source 1..41: mol_type = other DNA
aaaacgaaaaagcacctttagcgttaacttcctatctcact

SEQ ID NO: 380 - DNA, 16 bp, synthetic construct
  misc_feature 1..16: note = source = /note="Description of Artificial Sequence: Synthetic oligonucleotide"
  source 1..16: mol_type = other DNA
aatatcggtttggtca

SEQ ID NO: 381 - DNA, 41 bp, synthetic construct
  misc_feature 1..41: note = source = /note="Description of Artificial Sequence: Synthetic oligonucleotide"
  source 1..41: mol_type = other DNA
cagcttatggtgtctgtaaaacagtaacttcctatctcact

SEQ ID NO: 382 - DNA, 16 bp, synthetic construct
  misc_feature 1..16: note = source = /note="Description of Artificial Sequence: Synthetic oligonucleotide"
  source 1..16: mol_type = other DNA
tgacgatgcaaaaatt

SEQ ID NO: 383 - DNA, 25 bp, synthetic construct
  misc_feature 1..25: note = source = /note="Description of Artificial Sequence: Synthetic oligonucleotide"
  source 1..25: mol_type = other DNA
accagcagaggaagcatcagcacca

SEQ ID NO: 384 - DNA, 25 bp, synthetic construct
  misc_feature 1..25: note = source = /note="Description of Artificial Sequence: Synthetic oligonucleotide"
  source 1..25: mol_type = other DNA
gtttttgagatggcagcaacggaaa

SEQ ID NO: 385 - DNA, 25 bp, synthetic construct
  misc_feature 1..25: note = source = /note="Description of Artificial Sequence: Synthetic oligonucleotide"
  source 1..25: mol_type = other DNA
acatacgaaggcgcataacgatacc

SEQ ID NO: 386 - DNA, 25 bp, synthetic construct
  misc_feature 1..25: note = source = /note="Description of Artificial Sequence: Synthetic oligonucleotide"
  source 1..25: mol_type = other DNA
gggtcggcatcaaaagcaatatcag

SEQ ID NO: 387 - DNA, 25 bp, synthetic construct
  misc_feature 1..25: note = source = /note="Description of Artificial Sequence: Synthetic oligonucleotide"
  source 1..25: mol_type = other DNA
gcaagataatcacgagtatcctttc

SEQ ID NO: 388 - DNA, 25 bp, synthetic construct
  misc_feature 1..25: note = source = /note="Description of Artificial Sequence: Synthetic oligonucleotide"
  source 1..25: mol_type = other DNA
ttagcctcggtacggtcaggcatcc

SEQ ID NO: 389 - DNA, 25 bp, synthetic construct
  misc_feature 1..25: note = source = /note="Description of Artificial Sequence: Synthetic oligonucleotide"
  source 1..25: mol_type = other DNA
caccagaacggaaaacatccttcat

SEQ ID NO: 390 - DNA, 25 bp, synthetic construct
  misc_feature 1..25: note = source = /note="Description of Artificial Sequence: Synthetic oligonucleotide"
  source 1..25: mol_type = other DNA
atgtatccatctgaatgcaatgaag

SEQ ID NO: 391 - DNA, 32 bp, synthetic construct
  misc_feature 1..32: note = source = /note="Description of Artificial Sequence: Synthetic oligonucleotide"
  source 1..32: mol_type = other DNA
gttgaccacctacataccaaatacgctcctga

SEQ ID NO: 392 - DNA, 50 bp, synthetic construct
  misc_feature 1..50: note = source = /note="Description of Artificial Sequence: Synthetic oligonucleotide"
  source 1..50: mol_type = other DNA
cgagagtcaggtacggcggtcagtagtttgttactcgtcagaaaatcgaa

SEQ ID NO: 393 - DNA, 28 bp, synthetic construct
  misc_feature 1..28: note = source = /note="Description of Artificial Sequence: Synthetic oligonucleotide"
  source 1..28: mol_type = other DNA
tgcgctggtaataagacgaccaggaatt

SEQ ID NO: 394 - DNA, 40 bp, synthetic construct
  misc_feature 1..40: note = source = /note="Description of Artificial Sequence: Synthetic oligonucleotide"
  source 1..40: mol_type = other DNA
gttaatcgagcggcatggtcaatattaacaccaaacgtga

SEQ ID NO: 395 - DNA, 40 bp, synthetic construct
  misc_feature 1..40: note = source = /note="Description of Artificial Sequence: Synthetic oligonucleotide"
  source 1..40: mol_type = other DNA
tctcctcactaccatgaacaaaatgtaatccacgctcttt

SEQ ID NO: 396 - DNA, 36 bp, synthetic construct
  misc_feature 1..36: note = source = /note="Description of Artificial Sequence: Synthetic oligonucleotide"
  source 1..36: mol_type = other DNA
ggaagacggcagcaataaacagtagttgaaagtaca

SEQ ID NO: 397 - DNA, 32 bp, synthetic construct
  misc_feature 1..32: note = source = /note="Description of Artificial Sequence: Synthetic oligonucleotide"
  source 1..32: mol_type = other DNA
taaaaagtgagagcgcgaaggagtcaaggaag

SEQ ID NO: 398 - DNA, 38 bp, synthetic construct
  misc_feature 1..38: note = source = /note="Description of Artificial Sequence: Synthetic oligonucleotide"
  source 1..38: mol_type = other DNA
taaaatgtcaacaaaccaatccaagagaatcttccaac

SEQ ID NO: 399 - DNA, 48 bp, synthetic construct
  misc_feature 1..48: note = source = /note="Description of Artificial Sequence: Synthetic oligonucleotide"
  source 1..48: mol_type = other DNA
atcatcttattagggttagcctcggcatccacggcgctttgtatcagg

SEQ ID NO: 400 - DNA, 46 bp, synthetic construct
  misc_feature 1..46: note = source = /note="Description of Artificial Sequence: Synthetic oligonucleotide"
  source 1..46: mol_type = other DNA
gcgtcagcgaaacgcagtttttgacatatcctacaaagtccaagca

SEQ ID NO: 401 - DNA, 36 bp, synthetic construct
  misc_feature 1..36: note = source = /note="Description of Artificial Sequence: Synthetic oligonucleotide"
  source 1..36: mol_type = other DNA
gtggagcttgcagacccatatcgttctctaacccag

SEQ ID NO: 402 - DNA, 28 bp, synthetic construct
  misc_feature 1..28: note = source = /note="Description of Artificial Sequence: Synthetic oligonucleotide"
  source 1..28: mol_type = other DNA
agatgaaaccatttttgaatctctcaga

SEQ ID NO: 403 - DNA, 40 bp, synthetic construct
  misc_feature 1..40: note = source = /note="Description of Artificial Sequence: Synthetic oligonucleotide"
  source 1..40: mol_type = other DNA
cggtcgtcagagtgtcaaaaacgatcatcaaaagtggagg

SEQ ID NO: 404 - DNA, 46 bp, synthetic construct
  misc_feature 1..46: note = source = /note="Description of Artificial Sequence: Synthetic oligonucleotide"
  source 1..46: mol_type = other DNA
ataactccaaagcagcctgaaacaaagtcatttggcgagaaaagat

SEQ ID NO: 405 - DNA, 32 bp, synthetic construct
  misc_feature 1..32: note = source = /note="Description of Artificial Sequence: Synthetic oligonucleotide"
  source 1..32: mol_type = other DNA
ttgaacacagagggcgcggcaaaagtggtcta

SEQ ID NO: 406 - DNA, 32 bp, synthetic construct
  misc_feature 1..32: note = source = /note="Description of Artificial Sequence: Synthetic oligonucleotide"
  source 1..32: mol_type = other DNA
agcagccttatggccgtcaaggagcacatcgg

SEQ ID NO: 407 - DNA, 38 bp, synthetic construct
  misc_feature 1..38: note = source = /note="Description of Artificial Sequence: Synthetic oligonucleotide"
  source 1..38: mol_type = other DNA
cagaagcctgaatgagggaaaccataacgagcattcaa

SEQ ID NO: 408 - DNA, 50 bp, synthetic construct
  misc_feature 1..50: note = source = /note="Description of Artificial Sequence: Synthetic oligonucleotide"
  source 1..50: mol_type = other DNA
ttgagttgcgtagccatggcagcaaccttaatagaggccaaagcggtctg

SEQ ID NO: 409 - DNA, 40 bp, synthetic construct
  misc_feature 1..40: note = source = /note="Description of Artificial Sequence: Synthetic oligonucleotide"
  source 1..40: mol_type = other DNA
ttatagatatcatcttgattaagcgttaaatccaaaacgg

SEQ ID NO: 410 - DNA, 48 bp, synthetic construct
  misc_feature 1..48: note = source = /note="Description of Artificial Sequence: Synthetic oligonucleotide"
  source 1..48: mol_type = other DNA
gaaacgtatcggcgtgtgaatcataccctcggcagcaagacctctaat

SEQ ID NO: 411 - DNA, 38 bp, synthetic construct
  misc_feature 1..38: note = source = /note="Description of Artificial Sequence: Synthetic oligonucleotide"
  source 1..38: mol_type = other DNA
gactcatgatttcttaaccatactcaggcacacgaaaa

SEQ ID NO: 412 - DNA, 28 bp, synthetic construct
  misc_feature 1..28: note = source = /note="Description of Artificial Sequence: Synthetic oligonucleotide"
  source 1..28: mol_type = other DNA
ccaactgcgccgccaagggaagtacgtg

SEQ ID NO: 413 - DNA, 46 bp, synthetic construct
  misc_feature 1..46: note = source = /note="Description of Artificial Sequence: Synthetic oligonucleotide"
  source 1..46: mol_type = other DNA
tagtcatggaaccgcacgcaaagcatggtcaacgctacctgtcgac

SEQ ID NO: 414 - DNA, 40 bp, synthetic construct
  misc_feature 1..40: note = source = /note="Description of Artificial Sequence: Synthetic oligonucleotide"
  source 1..40: mol_type = other DNA
caatatcacaaaaatactgatagcgattgttcagtaactt

SEQ ID NO: 415 - DNA, 48 bp, synthetic construct
  misc_feature 1..48: note = source = /note="Description of Artificial Sequence: Synthetic oligonucleotide"
  source 1..48: mol_type = other DNA
gactcagagactcatatctaaaccgaacgtgccaagcatagaaaggtc

SEQ ID NO: 416 - DNA, 32 bp, synthetic construct
  misc_feature 1..32: note = source = /note="Description of Artificial Sequence: Synthetic oligonucleotide"
  source 1..32: mol_type = other DNA
gacagatttgtcattgtgagtgcgttctctac

SEQ ID NO: 417 - DNA, 36 bp, synthetic construct
  misc_feature 1..36: note = source = /note="Description of Artificial Sequence: Synthetic oligonucleotide"
  source 1..36: mol_type = other DNA
gacctttaactggcggcgattcgtcacaggtagcca

SEQ ID NO: 418 - DNA, 32 bp, synthetic construct
  misc_feature 1..32: note = source = /note="Description of Artificial Sequence: Synthetic oligonucleotide"
  source 1..32: mol_type = other DNA
atcagaaaatacgccacgcgcataacttttcc

SEQ ID NO: 419 - DNA, 50 bp, synthetic construct
  misc_feature 1..50: note = source = /note="Description of Artificial Sequence: Synthetic oligonucleotide"
  source 1..50: mol_type = other DNA
gacggttgacagtccccattagctgtcctattagtggttgaacagcatcg

SEQ ID NO: 420 - DNA, 40 bp, synthetic construct
  misc_feature 1..40: note = source = /note="Description of Artificial Sequence: Synthetic oligonucleotide"
  source 1..40: mol_type = other DNA
gcaaagtagagctgcgcaaggatacgatttaatcaagata

SEQ ID NO: 421 - DNA, 42 bp, synthetic construct
  misc_feature 1..42: note = source = /note="Description of Artificial Sequence: Synthetic oligonucleotide"
  source 1..42: mol_type = other DNA
aagcatgtcaatgctcagtctctttttcatcttcacactact

SEQ ID NO: 422 - DNA, 41 bp, synthetic construct
  misc_feature 1..41: note = source = /note="Description of Artificial Sequence: Synthetic oligonucleotide"
  source 1..41: mol_type = other DNA
ttacctccaaatgaagaataattattcatcttcacactact

SEQ ID NO: 423 - DNA, 48 bp, synthetic construct
  misc_feature 1..48: note = source = /note="Description of Artificial Sequence: Synthetic oligonucleotide"
  source 1..48: mol_type = other DNA
gacgggatacaattcagctcgacgtttttgactcatcttcacactact

SEQ ID NO: 424 - DNA, 48 bp, synthetic construct
  misc_feature 1..48: note = source = /note="Description of Artificial Sequence: Synthetic oligonucleotide"
  source 1..48: mol_type = other DNA
ggcgctgcgtaaccgtcttcctctacgcaatatcatcttcacactact

SEQ ID NO: 425 - DNA, 48 bp, synthetic construct
  misc_feature 1..48: note = source = /note="Description of Artificial Sequence: Synthetic oligonucleotide"
  source 1..48: mol_type = other DNA
taaggccaaagcggctaggccacagcaaggtctcatcttcacactact

SEQ ID NO: 426 - DNA, 36 bp, synthetic construct
  misc_feature 1..36: note = source = /note="Description of Artificial Sequence: Synthetic oligonucleotide"
  source 1..36: mol_type = other DNA
caatggcgccaggcaatggatcatcttcacactact

SEQ ID NO: 427 - DNA, 48 bp, synthetic construct
  misc_feature 1..48: note = source = /note="Description of Artificial Sequence: Synthetic oligonucleotide"
  source 1..48: mol_type = other DNA
ccagcgccagcgataaccggatccactgctactcatcttcacactact

SEQ ID NO: 428 - DNA, 60 bp, synthetic construct
  misc_feature 1..60: note = source = /note="Description of Artificial Sequence: Synthetic oligonucleotide"
  source 1..60: mol_type = other DNA
ttaagcgagcgccagaacgttttttaccgaacgtcactcaagtatcatcttcacactact

SEQ ID NO: 429 - DNA, 60 bp, synthetic construct
  misc_feature 1..60: note = source = /note="Description of Artificial Sequence: Synthetic oligonucleotide"
  source 1..60: mol_type = other DNA
actcgcgacagcttggtttttagtgagttttagccataacccagtcatcttcacactact

SEQ ID NO: 430 - DNA, 56 bp, synthetic construct
  misc_feature 1..56: note = source = /note="Description of Artificial Sequence: Synthetic oligonucleotide"
  source 1..56: mol_type = other DNA
tatcgtgtttcccgcaaggtaaacgcgagaacataaccattcatcttcacactact

SEQ ID NO: 431 - DNA, 42 bp, synthetic construct
  misc_feature 1..42: note = source = /note="Description of Artificial Sequence: Synthetic oligonucleotide"
  source 1..42: mol_type = other DNA
cttatcaacagggcgtaccagagtcatcatcttcacactact

SEQ ID NO: 432 - DNA, 60 bp, synthetic construct
  misc_feature 1..60: note = source = /note="Description of Artificial Sequence: Synthetic oligonucleotide"
  source 1..60: mol_type = other DNA
acgttccaagagctcaaagaaacgcggccatcaacactcagtaatcatcttcacactact

SEQ ID NO: 433 - DNA, 48 bp, synthetic construct
  misc_feature 1..48: note = source = /note="Description of Artificial Sequence: Synthetic oligonucleotide"
  source 1..48: mol_type = other DNA
agtaaatttgagcagatttgagtaattcagtgtcatcttcacactact

SEQ ID NO: 434 - DNA, 41 bp, synthetic construct
  misc_feature 1..41: note = source = /note="Description of Artificial Sequence: Synthetic oligonucleotide"
  source 1..41: mol_type = other DNA
ttaataatgttttccgtaaaggccatcatcttcacactact

SEQ ID NO: 435 - DNA, 56 bp, synthetic construct
  misc_feature 1..56: note = source = /note="Description of Artificial Sequence: Synthetic oligonucleotide"
  source 1..56: mol_type = other DNA
actttcatagccagattagagcgcatgagcaaattaaacgtcatcttcacactact

SEQ ID NO: 436 - DNA, 36 bp, synthetic construct
  misc_feature 1..36: note = source = /note="Description of Artificial Sequence: Synthetic oligonucleotide"
  source 1..36: mol_type = other DNA
tcgggcttcaataaactctgtcatcttcacactact

SEQ ID NO: 437 - DNA, 60 bp, synthetic construct
  misc_feature 1..60: note = source = /note="Description of Artificial Sequence: Synthetic oligonucleotide"
  source 1..60: mol_type = other DNA
ttaacgtatgttccatctttagcacttggtaagttggatttcgctcatcttcacactact

SEQ ID NO: 438 - DNA, 60 bp, synthetic construct
  misc_feature 1..60: note = source = /note="Description of Artificial Sequence: Synthetic oligonucleotide"
  source 1..60: mol_type = other DNA
gtcagttcacagaatgacacgacccagatacaaactcatcattatcatcttcacactact

SEQ ID NO: 439 - DNA, 60 bp, synthetic construct
  misc_feature 1..60: note = source = /note="Description of Artificial Sequence: Synthetic oligonucleotide"
  source 1..60: mol_type = other DNA
gctaaaaattaggagcctgtcgcattgcaaccaacccgatgggctcatcttcacactact

SEQ ID NO: 440 - DNA, 60 bp, synthetic construct
  misc_feature 1..60: note = source = /note="Description of Artificial Sequence: Synthetic oligonucleotide"
  source 1..60: mol_type = other DNA
gtcagtaatttagacagcacgtaaaggggccgaagcccctaatttcatcttcacactact

SEQ ID NO: 441 - DNA, 60 bp, synthetic construct
  misc_feature 1..60: note = source = /note="Description of Artificial Sequence: Synthetic oligonucleotide"
  source 1..60: mol_type = other DNA
caatagcgagggtccgccagcagtccactcgaatttagacaggctcatcttcacactact

SEQ ID NO: 442 - DNA, 36 bp, synthetic construct
  misc_feature 1..36: note = source = /note="Description of Artificial Sequence: Synthetic oligonucleotide"
  source 1..36: mol_type = other DNA
ccctttgtagcaatcgcgaatcatcttcacactact

SEQ ID NO: 443 - DNA, 60 bp, synthetic construct
  misc_feature 1..60: note = source = /note="Description of Artificial Sequence: Synthetic oligonucleotide"
  source 1..60: mol_type = other DNA
aagctagaagtcgtcgggagaggagtggaccagtagatgaagtatcatcttcacactact

SEQ ID NO: 444 - DNA, 42 bp, synthetic construct
  misc_feature 1..42: note = source = /note="Description of Artificial Sequence: Synthetic oligonucleotide"
  source 1..42: mol_type = other DNA
cagttgcgtaccaggaagtgcttctgtcatcttcacactact

SEQ ID NO: 445 - DNA, 41 bp, synthetic construct
  misc_feature 1..41: note = source = /note="Description of Artificial Sequence: Synthetic oligonucleotide"
  source 1..41: mol_type = other DNA
aaccagaacgtgaaaaagtggaagctcatcttcacactact

SEQ ID NO: 446 - DNA, 48 bp, synthetic construct
  misc_feature 1..48: note = source = /note="Description of Artificial Sequence: Synthetic oligonucleotide"
  source 1..48: mol_type = other DNA
gtcagtatcaagtaaacataagagagaaaacttcatcttcacactact

SEQ ID NO: 447 - DNA, 56 bp, synthetic construct
  misc_feature 1..56: note = source = /note="Description of Artificial Sequence: Synthetic oligonucleotide"
  source 1..56: mol_type = other DNA
gtcgcatagaaatataactggtagctttcgtattttgcgatcatcttcacactact

SEQ ID NO: 448 - DNA, 38 bp, synthetic construct
  misc_feature 1..38: note = source = /note="Description of Artificial Sequence: Synthetic oligonucleotide"
  source 1..38: mol_type = other DNA
cctacgagtcagtccttggggataacatcatggtgcat

SEQ ID NO: 449 - DNA, 50 bp, synthetic construct
  misc_feature 1..50: note = source = /note="Description of Artificial Sequence: Synthetic oligonucleotide"
  source 1..50: mol_type = other DNA
cctacgagtcagtcctcctttcaacgtgagccaggaggaagcggatgttt

SEQ ID NO: 450 - DNA, 40 bp, synthetic construct
  misc_feature 1..40: note = source = /note="Description of Artificial Sequence: Synthetic oligonucleotide"
  source 1..40: mol_type = other DNA
cctacgagtcagtcctgataatctggttgaacggcgaagc

SEQ ID NO: 451 - DNA, 40 bp, synthetic construct
  misc_feature 1..40: note = source = /note="Description of Artificial Sequence: Synthetic oligonucleotide"
  source 1..40: mol_type = other DNA
cctacgagtcagtcctactcttgtgccaattcatccacga

SEQ ID NO: 452 - DNA, 50 bp, synthetic construct
  misc_feature 1..50: note = source = /note="Description of Artificial Sequence: Synthetic oligonucleotide"
  source 1..50: mol_type = other DNA
cctacgagtcagtcctagtataaagaaatgccaacgcaagcctccgagca

SEQ ID NO: 453 - DNA, 50 bp, synthetic construct
  misc_feature 1..50: note = source = /note="Description of Artificial Sequence: Synthetic oligonucleotide"
  source 1..50: mol_type = other DNA
cctacgagtcagtcctttaatccttctcagagcgcataaagtgcaatgaa

SEQ ID NO: 454 - DNA, 60 bp, synthetic construct
  misc_feature 1..60: note = source = /note="Description of Artificial Sequence: Synthetic oligonucleotide"
  source 1..60: mol_type = other DNA
cctacgagtcagtcctaagttgggcatacatacaacgccctgcatacgaaaagacacgtc

SEQ ID NO: 455 - DNA, 60 bp, synthetic construct
  misc_feature 1..60: note = source = /note="Description of Artificial Sequence: Synthetic oligonucleotide"
  source 1..60: mol_type = other DNA
cctacgagtcagtcctcatgcggcagacgagcttagtacctcgcaacggctgcggacgac

SEQ ID NO: 456 - DNA, 40 bp, synthetic construct
  misc_feature 1..40: note = source = /note="Description of Artificial Sequence: Synthetic oligonucleotide"
  source 1..40: mol_type = other DNA
cctacgagtcagtcctcgactttgaatattagacatgcaa

SEQ ID NO: 457 - DNA, 60 bp, synthetic construct
  misc_feature 1..60: note = source = /note="Description of Artificial Sequence: Synthetic oligonucleotide"
  source 1..60: mol_type = other DNA
cctacgagtcagtccttctcttttcattttcacattctgattctgaacagcttcttaacg

SEQ ID NO: 458 - DNA, 52 bp, synthetic construct
  misc_feature 1..52: note = source = /note="Description of Artificial Sequence: Synthetic oligonucleotide"
  source 1..52: mol_type = other DNA
cctacgagtcagtcctactgatcagcattgggattatcataaaacatacgac

SEQ ID NO: 459 - DNA, 38 bp, synthetic construct
  misc_feature 1..38: note = source = /note="Description of Artificial Sequence: Synthetic oligonucleotide"
  source 1..38: mol_type = other DNA
cctacgagtcagtcctcgtcacgtcctgcgtgtagcaa

SEQ ID NO: 460 - DNA, 52 bp, synthetic construct
  misc_feature 1..52: note = source = /note="Description of Artificial Sequence: Synthetic oligonucleotide"
  source 1..52: mol_type = other DNA
cctacgagtcagtccttatccttatcatccttgatttcatcgctgaatagca

SEQ ID NO: 461 - DNA, 52 bp, synthetic construct
  misc_feature 1..52: note = source = /note="Description of Artificial Sequence: Synthetic oligonucleotide"
  source 1..52: mol_type = other DNA
cctacgagtcagtcctgatgtctcatttgaatcgttagttgatgaagccact

SEQ ID NO: 462 - DNA, 52 bp, synthetic construct
  misc_feature 1..52: note = source = /note="Description of Artificial Sequence: Synthetic oligonucleotide"
  source 1..52: mol_type = other DNA
cctacgagtcagtcctcaggttggtatccgaactgctttattcgtaaacaag

SEQ ID NO: 463 - DNA, 52 bp, synthetic construct
  misc_feature 1..52: note = source = /note="Description of Artificial Sequence: Synthetic oligonucleotide"
  source 1..52: mol_type = other DNA
cctacgagtcagtcctctgctgttaacatgcttagggattttataatagttg

SEQ ID NO: 464 - DNA, 52 bp, synthetic construct
  misc_feature 1..52: note = source = /note="Description of Artificial Sequence: Synthetic oligonucleotide"
  source 1..52: mol_type = other DNA
cctacgagtcagtcctgaaagacgaaaaatgtttcaccatatccttcatgaa

SEQ ID NO: 465 - DNA, 38 bp, synthetic construct
  misc_feature 1..38: note = source = /note="Description of Artificial Sequence: Synthetic oligonucleotide"
  source 1..38: mol_type = other DNA
cctacgagtcagtcctatagcaattcagcgcctttaag

SEQ ID NO: 466 - DNA, 36 bp, synthetic construct
  misc_feature 1..36: note = source = /note="Description of Artificial Sequence: Synthetic oligonucleotide"
  source 1..36: mol_type = other DNA
gttcagaaaactggcctaaccactgcaacaagaaaa

SEQ ID NO: 467 - DNA, 50 bp, synthetic construct
  misc_feature 1..50: note = source = /note="Description of Artificial Sequence: Synthetic oligonucleotide"
  source 1..50: mol_type = other DNA
gcgtcacctgaaacaataagaggttttgaagaaataacatcatggtaacg

SEQ ID NO: 468 - DNA, 40 bp, synthetic construct
  misc_feature 1..40: note = source = /note="Description of Artificial Sequence: Synthetic oligonucleotide"
  source 1..40: mol_type = other DNA
aaagcggcatagatattcaaataagtacggtcaggcatcc

SEQ ID NO: 469 - DNA, 28 bp, synthetic construct
  misc_feature 1..28: note = source = /note="Description of Artificial Sequence: Synthetic oligonucleotide"
  source 1..28: mol_type = other DNA
aggcgctgaacggacttcatagaagcgc

SEQ ID NO: 470 - DNA, 40 bp, synthetic construct
  misc_feature 1..40: note = source = /note="Description of Artificial Sequence: Synthetic oligonucleotide"
  source 1..40: mol_type = other DNA
tataacgtgctttaggtgtctgtagaaccagcaataaaag

SEQ ID NO: 471 - DNA, 48 bp, synthetic construct
  misc_feature 1..48: note = source = /note="Description of Artificial Sequence: Synthetic oligonucleotide"
  source 1..48: mol_type = other DNA
ctgcatgaatatcagcaccaacagattagcggcgttgacatatcaaaa

SEQ ID NO: 472 - DNA, 38 bp, synthetic construct
  misc_feature 1..38: note = source = /note="Description of Artificial Sequence: Synthetic oligonucleotide"
  source 1..38: mol_type = other DNA
acggcgctttaccaaatacctaaatagttgttatggtc

SEQ ID NO: 473 - DNA, 32 bp, synthetic construct
  misc_feature 1..32: note = source = /note="Description of Artificial Sequence: Synthetic oligonucleotide"
  source 1..32: mol_type = other DNA
ttaaactcaaaattttacattaaaatcatggt

SEQ ID NO: 474 - DNA, 46 bp, synthetic construct
  misc_feature 1..46: note = source = /note="Description of Artificial Sequence: Synthetic oligonucleotide"
  source 1..46: mol_type = other DNA
aatatgacggtaaagaaccagtagtgtcatagccagatgcccgtca

SEQ ID NO: 475 - DNA, 32 bp, synthetic construct
  misc_feature 1..32: note = source = /note="Description of Artificial Sequence: Synthetic oligonucleotide"
  source 1..32: mol_type = other DNA
ataacgataccactgaccctaatcaccatggt

SEQ ID NO: 476 - DNA, 46 bp, synthetic construct
  misc_feature 1..46: note = source = /note="Description of Artificial Sequence: Synthetic oligonucleotide"
  source 1..46: mol_type = other DNA
agaaaaaccggtcggaccaccattaccatccaaaggataaacccac

SEQ ID NO: 477 - DNA, 40 bp, synthetic construct
  misc_feature 1..40: note = source = /note="Description of Artificial Sequence: Synthetic oligonucleotide"
  source 1..40: mol_type = other DNA
tctgaatggggtcggcatcaaaagtaatcacgttcttggt

SEQ ID NO: 478 - DNA, 28 bp, synthetic construct
  misc_feature 1..28: note = source = /note="Description of Artificial Sequence: Synthetic oligonucleotide"
  source 1..28: mol_type = other DNA
accaaaaaccatttttgaatctctcaga

SEQ ID NO: 479 - DNA, 40 bp, synthetic construct
  misc_feature 1..40: note = source = /note="Description of Artificial Sequence: Synthetic oligonucleotide"
  source 1..40: mol_type = other DNA
cagcaagacaatatcacgaaaatataatcggtaaccaacc

SEQ ID NO: 480 - DNA, 38 bp, synthetic construct
  misc_feature 1..38: note = source = /note="Description of Artificial Sequence: Synthetic oligonucleotide"
  source 1..38: mol_type = other DNA
cagtatgcaaattagcacaggcaaaaaatttacaatga

SEQ ID NO: 481 - DNA, 36 bp, synthetic construct
  misc_feature 1..36: note = source = /note="Description of Artificial Sequence: Synthetic oligonucleotide"
  source 1..36: mol_type = other DNA
catcacacagtccttgacggtcgttctctaatccgc

SEQ ID NO: 482 - DNA, 32 bp, synthetic construct
  misc_feature 1..32: note = source = /note="Description of Artificial Sequence: Synthetic oligonucleotide"
  source 1..32: mol_type = other DNA
ctgtcgcacctccagccggcaaaagtggtcta

SEQ ID NO: 483 - DNA, 50 bp, synthetic construct
  misc_feature 1..50: note = source = /note="Description of Artificial Sequence: Synthetic oligonucleotide"
  source 1..50: mol_type = other DNA
actcagaagagaaataagcgaaccaaataagcagcttgcagacccataat

SEQ ID NO: 484 - DNA, 32 bp, synthetic construct; identical to SEQ ID NO: 406
  misc_feature 1..32: note = source = /note="Description of Artificial Sequence: Synthetic oligonucleotide"
  source 1..32: mol_type = other DNA

SEQ ID NO: 485 - DNA, 48 bp, synthetic construct
  misc_feature 1..48: note = source = /note="Description of Artificial Sequence: Synthetic oligonucleotide"
  source 1..48: mol_type = other DNA
gtcaatagtaaagtgcaccgcatgcggccattagctgtacaccctcgg

SEQ ID NO: 486 - DNA, 50 bp, synthetic construct
  misc_feature 1..50: note = source = /note="Description of Artificial Sequence: Synthetic oligonucleotide"
  source 1..50: mol_type = other DNA
tacccttgaaagtgggacgaccaaaaagtctcaggaggaagcggagcagt

SEQ ID NO: 487 - DNA, 36 bp, synthetic construct
  misc_feature 1..36: note = source = /note="Description of Artificial Sequence: Synthetic oligonucleotide"
  source 1..36: mol_type = other DNA
tgcggtgaaaaagcgtcctgagcgcgcataaacgta

SEQ ID NO: 488 - DNA, 40 bp, synthetic construct
  misc_feature 1..40: note = source = /note="Description of Artificial Sequence: Synthetic oligonucleotide"
  source 1..40: mol_type = other DNA
cacacaaactgtaggaagtgtccggtggtagaagtcgtca

SEQ ID NO: 489 - DNA, 32 bp, synthetic construct
  misc_feature 1..32: note = source = /note="Description of Artificial Sequence: Synthetic oligonucleotide"
  source 1..32: mol_type = other DNA
gataggtcgtagtaatggatacgcgcgccgcc

SEQ ID NO: 490 - DNA, 46 bp, synthetic construct
  misc_feature 1..46: note = source = /note="Description of Artificial Sequence: Synthetic oligonucleotide"
  source 1..46: mol_type = other DNA
gatagcggcgactggcagtcggcgtgactgtaaccataaggcgaac

SEQ ID NO: 491 - DNA, 40 bp, synthetic construct
  misc_feature 1..40: note = source = /note="Description of Artificial Sequence: Synthetic oligonucleotide"
  source 1..40: mol_type = other DNA
tctaaaccgaacgtgccaagcatagacagaatagcttctc

SEQ ID NO: 492 - DNA, 32 bp, synthetic construct
  misc_feature 1..32: note = source = /note="Description of Artificial Sequence: Synthetic oligonucleotide"
  source 1..32: mol_type = other DNA
tggtaagttggattaagcaccccagcctcaca

SEQ ID NO: 493 - DNA, 38 bp, synthetic construct
  misc_feature 1..38: note = source = /note="Description of Artificial Sequence: Synthetic oligonucleotide"
  source 1..38: mol_type = other DNA
tttggcgagaaagctcttagggtcaacgctacaatact

SEQ ID NO: 494 - DNA, 28 bp, synthetic construct
  misc_feature 1..28: note = source = /note="Description of Artificial Sequence: Synthetic oligonucleotide"
  source 1..28: mol_type = other DNA
tgttaatttgagcagactcattctagct

SEQ ID NO: 495 - DNA, 48 bp, synthetic construct
  misc_feature 1..48: note = source = /note="Description of Artificial Sequence: Synthetic oligonucleotide"
  source 1..48: mol_type = other DNA
ccaaatgttgatttcttacctattcagcatcggactcagagactcata

SEQ ID NO: 496 - DNA, 48 bp, synthetic construct
  misc_feature 1..48: note = source = /note="Description of Artificial Sequence: Synthetic oligonucleotide"
  source 1..48: mol_type = other DNA
gttagcctctgaaacaaatgcttatggtatcagggttaatgtggcatt

SEQ ID NO: 497 - DNA, 46 bp, synthetic construct
  misc_feature 1..46: note = source = /note="Description of Artificial Sequence: Synthetic oligonucleotide"
  source 1..46: mol_type = other DNA
gtcaaatgagcctgacaagagaatctatcgaaatcatcttcgtgtt

SEQ ID NO: 498 - DNA, 32 bp, synthetic construct
  misc_feature 1..32: note = source = /note="Description of Artificial Sequence: Synthetic oligonucleotide"
  source 1..32: mol_type = other DNA
tttcttctgcgtcagtaagaaccagggccttt

SEQ ID NO: 499 - DNA, 38 bp, synthetic construct
  misc_feature 1..38: note = source = /note="Description of Artificial Sequence: Synthetic oligonucleotide"
  source 1..38: mol_type = other DNA
ggaaaccataacgagctggaaacgtacggatttaaaat

SEQ ID NO: 500 - DNA, 32 bp, synthetic construct
  misc_feature 1..32: note = source = /note="Description of Artificial Sequence: Synthetic oligonucleotide"
  source 1..32: mol_type = other DNA
acggcgtcaccaatctgccgaagcttgccttt

SEQ ID NO: 501 - DNA, 40 bp, synthetic construct
  misc_feature 1..40: note = source = /note="Description of Artificial Sequence: Synthetic oligonucleotide"
  source 1..40: mol_type = other DNA
cgctctttgttcagtaacttgactttgagatggcagcaac

SEQ ID NO: 502 - DNA, 50 bp, synthetic construct
  misc_feature 1..50: note = source = /note="Description of Artificial Sequence: Synthetic oligonucleotide"
  source 1..50: mol_type = other DNA
agaggtttatgggatccaaagcggtcatcatcttgattaagctcattagg

SEQ ID NO: 503 - DNA, 40 bp, synthetic construct
  misc_feature 1..40: note = source = /note="Description of Artificial Sequence: Synthetic oligonucleotide"
  source 1..40: mol_type = other DNA
aacaccatcttaatccactgttcaatgtctacgagaaaga

SEQ ID NO: 504 - DNA, 28 bp, synthetic construct
  misc_feature 1..28: note = source = /note="Description of Artificial Sequence: Synthetic oligonucleotide"
  source 1..28: mol_type = other DNA
cggcaacataccaaaggcggctttacgt

SEQ ID NO: 505 - DNA, 36 bp, synthetic construct
  misc_feature 1..36: note = source = /note="Description of Artificial Sequence: Synthetic oligonucleotide"
  source 1..36: mol_type = other DNA
actcagcggtcagtagcaatgttgaccacctgcaat

SEQ ID NO: 506 - DNA, 44 bp, synthetic construct
  misc_feature 1..44: note = source = /note="Description of Artificial Sequence: Synthetic oligonucleotide"
  source 1..44: mol_type = other DNA
cagcaaggtagcgacagttgttccttgaacggcgtcgcgtctgc

SEQ ID NO: 507 - DNA, 26 bp, synthetic construct
  misc_feature 1..26: note = source = /note="Description of Artificial Sequence: Synthetic oligonucleotide"
  source 1..26: mol_type = other DNA
agcaccaaacttgttaaatcccagcg

SEQ ID NO: 508 - DNA, 44 bp, synthetic construct
  misc_feature 1..44: note = source = /note="Description of Artificial Sequence: Synthetic oligonucleotide"
  source 1..44: mol_type = other DNA
gtactgaatacaaaactcggtatagataagcaggagaaacaagc

SEQ ID NO: 509 - DNA, 32 bp, synthetic construct
  misc_feature 1..32: note = source = /note="Description of Artificial Sequence: Synthetic oligonucleotide"
  source 1..32: mol_type = other DNA
caacaggcgcgaatatctttttggattcttta

SEQ ID NO: 510 - DNA, 20 bp, synthetic construct
  misc_feature 1..20: note = source = /note="Description of Artificial Sequence: Synthetic oligonucleotide"
  source 1..20: mol_type = other DNA
gcgcgagcgccaaaatggta

SEQ ID NO: 511 - DNA, 44 bp, synthetic construct
  misc_feature 1..44: note = source = /note="Description of Artificial Sequence: Synthetic oligonucleotide"
  source 1..44: mol_type = other DNA
tctttaatccagcaatcacctcaccagatacaaactcatcatta

SEQ ID NO: 512 - DNA, 44 bp, synthetic construct
  misc_feature 1..44: note = source = /note="Description of Artificial Sequence: Synthetic oligonucleotide"
  source 1..44: mol_type = other DNA
gaacatgggcatattatcataaaacgcccacgcaaaaagcggct

SEQ ID NO: 513 - DNA, 47 bp, synthetic construct
  misc_feature 1..47: note = source = /note="Description of Artificial Sequence: Synthetic oligonucleotide"
  source 1..47: mol_type = other DNA
gaagatttcacgcggcggcaagttgccatctctttaaagaaggtcat

SEQ ID NO: 514 - DNA, 32 bp, synthetic construct
  misc_feature 1..32: note = source = /note="Description of Artificial Sequence: Synthetic oligonucleotide"
  source 1..32: mol_type = other DNA
gtacggggtgaatcgcaatcttttttaagtgg

SEQ ID NO: 515 - DNA, 26 bp, synthetic construct
  misc_feature 1..26: note = source = /note="Description of Artificial Sequence: Synthetic oligonucleotide"
  source 1..26: mol_type = other DNA
aagtgacgtttgagagattaccgcgc

SEQ ID NO: 516 - DNA, 25 bp, synthetic construct
  misc_feature 1..25: note = source = /note="Description of Artificial Sequence: Synthetic oligonucleotide"
  source 1..25: mol_type = other DNA
acgaagtagcggtaaagtagcctct

SEQ ID NO: 517 - DNA, 40 bp, synthetic construct
  misc_feature 1..40: note = source = /note="Description of Artificial Sequence: Synthetic oligonucleotide"
  source 1..40: mol_type = other DNA
cttcgtcgcagtcgaacaagcggcgccatctgttgacagg

SEQ ID NO: 518 - DNA, 32 bp, synthetic construct
  misc_feature 1..32: note = source = /note="Description of Artificial Sequence: Synthetic oligonucleotide"
  source 1..32: mol_type = other DNA
tttatagggtttgaattcatgcggagtcaaag

SEQ ID NO: 519 - DNA, 44 bp, synthetic construct
  misc_feature 1..44: note = source = /note="Description of Artificial Sequence: Synthetic oligonucleotide"
  source 1..44: mol_type = other DNA
agtcatggcgacctggagtaacagaagtacaggtgccataaaca

SEQ ID NO: 520 - DNA, 20 bp, synthetic construct
  misc_feature 1..20: note = source = /note="Description of Artificial Sequence: Synthetic oligonucleotide"
  source 1..20: mol_type = other DNA
acacgaaggcggctcagcgg

SEQ ID NO: 521 - DNA, 25 bp, synthetic construct
  misc_feature 1..25: note = source = /note="Description of Artificial Sequence: Synthetic oligonucleotide"
  source 1..25: mol_type = other DNA
tccgacaggagcaggaaacggagta

SEQ ID NO: 522 - DNA, 40 bp, synthetic construct
  misc_feature 1..40: note = source = /note="Description of Artificial Sequence: Synthetic oligonucleotide"
  source 1..40: mol_type = other DNA
actttcagcgaaggcatttagtcatgataaggacgtgaaa

SEQ ID NO: 523 - DNA, 40 bp, synthetic construct
  misc_feature 1..40: note = source = /note="Description of Artificial Sequence: Synthetic oligonucleotide"
  source 1..40: mol_type = other DNA
ttcatgactttttttagccatactcatccacaaccagtag

SEQ ID NO: 524 - DNA, 44 bp, synthetic construct
  misc_feature 1..44: note = source = /note="Description of Artificial Sequence: Synthetic oligonucleotide"
  source 1..44: mol_type = other DNA
cgggatgaaatgttttttccatgaccttctgcacgtaatttaga

SEQ ID NO: 525 - DNA, 26 bp, synthetic construct
  misc_feature 1..26: note = source = /note="Description of Artificial Sequence: Synthetic oligonucleotide"
  source 1..26: mol_type = other DNA
gcgacgtgtagccacgtattcgccag

SEQ ID NO: 526 - DNA, 32 bp, synthetic construct
  misc_feature 1..32: note = source = /note="Description of Artificial Sequence: Synthetic oligonucleotide"
  source 1..32: mol_type = other DNA
ggttcaatcatttttatcgaaaggtcgctcat

SEQ ID NO: 527 - DNA, 25 bp, synthetic construct
  misc_feature 1..25: note = source = /note="Description of Artificial Sequence: Synthetic oligonucleotide"
  source 1..25: mol_type = other DNA
acgctcgcaagagtaaacttggtca

SEQ ID NO: 528 - DNA, 25 bp, synthetic construct
  misc_feature 1..25: note = source = /note="Description of Artificial Sequence: Synthetic oligonucleotide"
  source 1..25: mol_type = other DNA
aattacatagaaaccaaccacttcg

SEQ ID NO: 529 - DNA, 44 bp, synthetic construct
  misc_feature 1..44: note = source = /note="Description of Artificial Sequence: Synthetic oligonucleotide"
  source 1..44: mol_type = other DNA
gacccatcaacagggacataaaaagtaaataaacgtaagaaacg

SEQ ID NO: 530 - DNA, 47 bp, synthetic construct
  misc_feature 1..47: note = source = /note="Description of Artificial Sequence: Synthetic oligonucleotide"
  source 1..47: mol_type = other DNA
acgcaaccggacgctcgacgccattaatacataataacatcactata

SEQ ID NO: 531 - DNA, 32 bp, synthetic construct
  misc_feature 1..32: note = source = /note="Description of Artificial Sequence: Synthetic oligonucleotide"
  source 1..32: mol_type = other DNA
cctcaacgacttctgccatcagaatgagacag

SEQ ID NO: 532 - DNA, 44 bp, synthetic construct
  misc_feature 1..44: note = source = /note="Description of Artificial Sequence: Synthetic oligonucleotide"
  source 1..44: mol_type = other DNA
cgaggtcagaaatccaacgcgtcagtttaagccactcagcgtac

SEQ ID NO: 533 - DNA, 26 bp, synthetic construct
  misc_feature 1..26: note = source = /note="Description of Artificial Sequence: Synthetic oligonucleotide"
  source 1..26: mol_type = other DNA
tgagtataataaatcataggctgaat

SEQ ID NO: 534 - DNA, 47 bp, synthetic construct
  misc_feature 1..47: note = source = /note="Description of Artificial Sequence: Synthetic oligonucleotide"
  source 1..47: mol_type = other DNA
acccgattctgaacagcttcttgggaagtccatatcatatctggggt

SEQ ID NO: 535 - DNA, 40 bp, synthetic construct
  misc_feature 1..40: note = source = /note="Description of Artificial Sequence: Synthetic oligonucleotide"
  source 1..40: mol_type = other DNA
cattagcaatgaaaactcaaaagtgttacagcgacgacaa

SEQ ID NO: 536 - DNA, 20 bp, synthetic construct
  misc_feature 1..20: note = source = /note="Description of Artificial Sequence: Synthetic oligonucleotide"
  source 1..20: mol_type = other DNA
ccctttgtagcagatttcat

SEQ ID NO: 537 - DNA, 20 bp, synthetic construct
  misc_feature 1..20: note = source = /note="Description of Artificial Sequence: Synthetic oligonucleotide"
  source 1..20: mol_type = other DNA
tcgtcaatctcaaattcgta

SEQ ID NO: 538 - DNA, 32 bp, synthetic construct
  misc_feature 1..32: note = source = /note="Description of Artificial Sequence: Synthetic oligonucleotide"
  source 1..32: mol_type = other DNA
ggcgctgcgtaaccgtcttcagtgtcaaatca

SEQ ID NO: 539 - DNA, 47 bp, synthetic construct
  misc_feature 1..47: note = source = /note="Description of Artificial Sequence: Synthetic oligonucleotide"
  source 1..47: mol_type = other DNA
acgttccaagagctcagaagcaataccgaacctgatctcagtaaatc

SEQ ID NO: 540 - DNA, 32 bp, synthetic construct
  misc_feature 1..32: note = source = /note="Description of Artificial Sequence: Synthetic oligonucleotide"
  source 1..32: mol_type = other DNA
catatttaacctgactattcttgaattaaaaa

SEQ ID NO: 541 - DNA, 32 bp, synthetic construct
  misc_feature 1..32: note = source = /note="Description of Artificial Sequence: Synthetic oligonucleotide"
  source 1..32: mol_type = other DNA
acgcccctgcaattaaaattaggccacgagga

SEQ ID NO: 542 - DNA, 24 bp, synthetic construct
  misc_feature 1..24: note = source = /note="Description of Artificial Sequence: Synthetic oligonucleotide"
  source 1..24: mol_type = other DNA
acccttcctgaatgaatgggatac

SEQ ID NO: 543 - DNA, 24 bp, synthetic construct
  misc_feature 1..24: note = source = /note="Description of Artificial Sequence: Synthetic oligonucleotide"
  source 1..24: mol_type = other DNA
acgcttgtgccaattcatccacga

SEQ ID NO: 544 - DNA, 22 bp, synthetic construct
  misc_feature 1..22: note = source = /note="Description of Artificial Sequence: Synthetic oligonucleotide"
  source 1..22: mol_type = other DNA
cagtcagccatataactgacca

SEQ ID NO: 545 - DNA, 36 bp, synthetic construct
  misc_feature 1..36: note = source = /note="Description of Artificial Sequence: Synthetic oligonucleotide"
  source 1..36: mol_type = other DNA
agtggaggttgcattcaaacgatacgtcagccaacg

SEQ ID NO: 546 - DNA, 22 bp, synthetic construct
  misc_feature 1..22: note = source = /note="Description of Artificial Sequence: Synthetic oligonucleotide"
  source 1..22: mol_type = other DNA
agcaatagaccaaaccatcaat

SEQ ID NO: 547 - DNA, 22 bp, synthetic construct
  misc_feature 1..22: note = source = /note="Description of Artificial Sequence: Synthetic oligonucleotide"
  source 1..22: mol_type = other DNA
ttcgcatagtgccatgctacac

SEQ ID NO: 548 - DNA, 34 bp, synthetic construct
  misc_feature 1..34: note = source = /note="Description of Artificial Sequence: Synthetic oligonucleotide"
  source 1..34: mol_type = other DNA
gtcgcaaatgaaccttcaacggcagaagcttaat

SEQ ID NO: 549 - DNA, 34 bp, synthetic construct
  misc_feature 1..34: note = source = /note="Description of Artificial Sequence: Synthetic oligonucleotide"
  source 1..34: mol_type = other DNA
aacgcagacgacaccattcgggagggtaaagaag

SEQ ID NO: 550 - DNA, 36 bp, synthetic construct
  misc_feature 1..36: note = source = /note="Description of Artificial Sequence: Synthetic oligonucleotide"
  source 1..36: mol_type = other DNA
aacaagcagaattttcaaagtaagcgttagttgatg

SEQ ID NO: 551 - DNA, 44 bp, synthetic construct
  misc_feature 1..44: note = source = /note="Description of Artificial Sequence: Synthetic oligonucleotide"
  source 1..44: mol_type = other DNA
aagttgggcatacatacaacgccctgcatacgaaaagacacgtc

SEQ ID NO: 552 - DNA, 34 bp, synthetic construct
  misc_feature 1..34: note = source = /note="Description of Artificial Sequence: Synthetic oligonucleotide"
  source 1..34: mol_type = other DNA
tttcttgttgacagtcccaagctatttaattgcg

SEQ ID NO: 553 - DNA, 36 bp, synthetic construct
  misc_feature 1..36: note = source = /note="Description of Artificial Sequence: Synthetic oligonucleotide"
  source 1..36: mol_type = other DNA
caaaaattctaagcagtggcgagattatcagaaaaa

SEQ ID NO: 554 - DNA, 44 bp, synthetic construct
  misc_feature 1..44: note = source = /note="Description of Artificial Sequence: Synthetic oligonucleotide"
  source 1..44: mol_type = other DNA
ttcttgcacagcaatcaatcaccagaacggaaaacatcctggaa

SEQ ID NO: 555 - DNA, 34 bp, synthetic construct
  misc_feature 1..34: note = source = /note="Description of Artificial Sequence: Synthetic oligonucleotide"
  source 1..34: mol_type = other DNA
cctagagatgtatgacgcgcatgacaagttgtca

SEQ ID NO: 556 - DNA, 36 bp, synthetic construct
  misc_feature 1..36: note = source = /note="Description of Artificial Sequence: Synthetic oligonucleotide"
  source 1..36: mol_type = other DNA
ccaacgaagaagcagcattaaccgtcaatgtatcca

SEQ ID NO: 557 - DNA, 36 bp, synthetic construct
  misc_feature 1..36: note = source = /note="Description of Artificial Sequence: Synthetic oligonucleotide"
  source 1..36: mol_type = other DNA
ctttgcattgggtgaatcattagccttgtactcagg

SEQ ID NO: 558 - DNA, 36 bp, synthetic construct
  misc_feature 1..36: note = source = /note="Description of Artificial Sequence: Synthetic oligonucleotide"
  source 1..36: mol_type = other DNA
agtctctcctcactaccatgaacaaaatgtaatcca

SEQ ID NO: 559 - DNA, 24 bp, synthetic construct
  misc_feature 1..24: note = source = /note="Description of Artificial Sequence: Synthetic oligonucleotide"
  source 1..24: mol_type = other DNA
attttctctctttttgcgttcgta

SEQ ID NO: 560 - DNA, 36 bp, synthetic construct
  misc_feature 1..36: note = source = /note="Description of Artificial Sequence: Synthetic oligonucleotide"
  source 1..36: mol_type = other DNA
ataagacgcatctcgaacgcaatgagtagagtcaat

SEQ ID NO: 561 - DNA, 44 bp, synthetic construct
  misc_feature 1..44: note = source = /note="Description of Artificial Sequence: Synthetic oligonucleotide"
  source 1..44: mol_type = other DNA
taactttttccgtggaagcattttcatcccgaagttgcggtttg

SEQ ID NO: 562 - DNA, 44 bp, synthetic construct
  misc_feature 1..44: note = source = /note="Description of Artificial Sequence: Synthetic oligonucleotide"
  source 1..44: mol_type = other DNA
tgcggacgacgtcagtcgcaaggtaaacgcgaacaattcaacga

SEQ ID NO: 563 - DNA, 24 bp, synthetic construct
  misc_feature 1..24: note = source = /note="Description of Artificial Sequence: Synthetic oligonucleotide"
  source 1..24: mol_type = other DNA
gttggaacgttttttacctttttg

SEQ ID NO: 564 - DNA, 22 bp, synthetic construct
  misc_feature 1..22: note = source = /note="Description of Artificial Sequence: Synthetic oligonucleotide"
  source 1..22: mol_type = other DNA
ataacgcgagggtatcctagca

SEQ ID NO: 565 - DNA, 36 bp, synthetic construct
  misc_feature 1..36: note = source = /note="Description of Artificial Sequence: Synthetic oligonucleotide"
  source 1..36: mol_type = other DNA
aacagacgatgattaacagtcgggagagtgccaaga

SEQ ID NO: 566 - DNA, 25 bp, synthetic construct
  misc_feature 1..25: note = source = /note="Description of Artificial Sequence: Synthetic oligonucleotide"
  source 1..25: mol_type = other DNA
ccgcttcggcgttataacctcacac

SEQ ID NO: 567 - DNA, 25 bp, synthetic construct
  misc_feature 1..25: note = source = /note="Description of Artificial Sequence: Synthetic oligonucleotide"
  source 1..25: mol_type = other DNA
catggaagcgataaaactctgcagg

SEQ ID NO: 568 - DNA, 25 bp, synthetic construct
  misc_feature 1..25: note = source = /note="Description of Artificial Sequence: Synthetic oligonucleotide"
  source 1..25: mol_type = other DNA
tcttgaacactcatccttaatacct

SEQ ID NO: 569 - DNA, 25 bp, synthetic construct
  misc_feature 1..25: note = source = /note="Description of Artificial Sequence: Synthetic oligonucleotide"
  source 1..25: mol_type = other DNA
ctgctttatcaagataatttttcga

SEQ ID NO: 570 - DNA, 25 bp, synthetic construct
  misc_feature 1..25: note = source = /note="Description of Artificial Sequence: Synthetic oligonucleotide"
  source 1..25: mol_type = other DNA
ttaagagggcgttcagcagccagct

SEQ ID NO: 571 - DNA, 25 bp, synthetic construct
  misc_feature 1..25: note = source = /note="Description of Artificial Sequence: Synthetic oligonucleotide"
  source 1..25: mol_type = other DNA
tagacataatttatcctcaagtaag

SEQ ID NO: 572 - DNA, 25 bp, synthetic construct
  misc_feature 1..25: note = source = /note="Description of Artificial Sequence: Synthetic oligonucleotide"
  source 1..25: mol_type = other DNA
gtggtcggcagattgcgataaacgg

SEQ ID NO: 573 - DNA, 25 bp, synthetic construct
  misc_feature 1..25: note = source = /note="Description of Artificial Sequence: Synthetic oligonucleotide"
  source 1..25: mol_type = other DNA
ccagcaaggaagccaagatgggaaa

SEQ ID NO: 574 - DNA, 15 bp, synthetic construct
  misc_feature 1..15: note = source = /note="Description of Artificial Sequence: Synthetic oligonucleotide"
  source 1..15: mol_type = other DNA
ggactgactcgtagg

SEQ ID NO: 575 - DNA, 15 bp, synthetic construct
  misc_feature 1..15: note = source = /note="Description of Artificial Sequence: Synthetic oligonucleotide"
  source 1..15: mol_type = other DNA
agtagtgtgaagatg

(19) United States
(12) Patent Application Publication
(10) Pub. No.: US 2025/0369951 A1
(43) Pub. Date: Dec. 4, 2025

(54) MEMBRANE BOUND NUCLEIC ACID NANOPORES

(71) Applicant: UCL Business Ltd, London (GB)

(72) Inventors: Stefan HOWORKA, London (GB); Yongzheng XING, London (GB)

(73) Assignee: UCL Business Ltd, London (GB)

(21) Appl. No.: 19/220,597

(22) Filed: May 28, 2025

Related U.S. Application Data
(62) Division of application No. 17/265,244, filed on Feb. 2, 2021, now Pat. No. 12,344,891.

(30) Foreign Application Priority Data
Aug. 2, 2018 (GB) 1812615.1

(51) Int. Cl. G01N 33/487; B82Y 15/00

(52) U.S. Cl. CPC G01N 33/48721; B82Y 15/00

(57) ABSTRACT

A membrane-spanning nanopore is provided, the nanopore comprising:
a. one or more polynucleotide strands that provide a scaffold component; and
b. a plurality of polynucleotide strands that provide a plurality of staple components; wherein each of the plurality of staple components hybridise to the scaffold component;
wherein the orientation of a major portion of at least one polynucleotide strand comprised within the scaffold component is substantially parallel to a planar surface of a membrane as well as embedded within and substantially coplanar with the membrane;
wherein the nanopore defines a channel that is suitable for perforating the membrane;
the channel having a longitudinal axis extending along the centre thereof and a minimum internal dimension perpendicular to the longitudinal axis of at least about 3 nm. A membrane comprising the nanopore, biological sensors comprising the membrane and a method for molecular sensing using the membrane are also disclosed

### CROSS-REFERENCE TO RELATED APPLICATIONS

This application is a divisional under 35 U.S.C. § 121 of co-pending U.S. Ser. No. 17/265,244 filed Feb. 2, 2021, which is a 35 U.S.C. § 371 National Phase Entry Application of International Application No. PCT/GB2019/052181 filed Aug. 2, 2019, which designates the U.S. and claims benefit under 35 U.S.C. § 119 (a) of G.B. Provisional Application No. 1812615.1 filed Aug. 2, 2018, the contents of which are incorporated herein by reference in their entireties.

### SEQUENCE LISTING

The instant application contains a Sequence Listing which has been submitted in XML format via Patent Center and is hereby incorporated by reference in its entirety. Said XML copy, created on May 27, 2025, is named P48126WO_sequence.xml and is 792,624 bytes in size.

### FIELD OF THE INVENTION

The invention is in the field of nucleic acid-based nanostructures that can insert into membranes and function as nanopores. Such nanopores may find utility in the fields of chemical and biological sensors, nanoscale devices and molecular gating applications.

### BACKGROUND OF THE INVENTION

Nanopores are membrane spanning polymers and complexes that can define a perforation and thereby form a channel in a membrane that forms a partition between two fluids, typically liquids, suitably aqueous solutions, through which ions and certain molecules may pass. The minimum diameter of the channel is typically in the nanometre (10-9 metre) range hence giving certain of these polypeptides the name ‘nanopores’. The channel typically is formed in a perpendicular orientation relative to the planar axis of the membrane and can be formed from proteins, peptides, synthetic organic compounds or nucleic acids, such as DNA (Howorka, Nature Nanotech., 12, July; 619-630 (2017)).

Nanopores bound in membranes have many potential uses. One example is the use of nanopores as sensors to analyse biomolecules in a label-free and portable fashion. In an embodiment of this approach, an electrical potential is applied across a membrane-bound nanopore causing ions to flow through the channel. This flow of ions can be measured as an electrical current. Suitable electrical measurement techniques using single channel recording have been described, for example, in WO 2000/28312 and D. Stoddart et al., Proc. Natl. Acad. Sci., 2010, 106, 7702-7. Multi-channel recording techniques have also been described, for example, in WO 2009/077734 and International Application WO-2011/067559. Optical measurements may be combined with electrical measurements (Soni G V et al., Rev Sci Instrum. 2010 January; 81 (1): 014301). Alternatively flow of ions through the membrane-bound nanopore may be achieved by providing an ionic gradient across the membrane. A measured electrical current can be used to assess of the size or degree of obstruction of the channel. These changes in electrical current can be used to identify that a molecule, or part of a molecule, has bound at or near the pore (molecular sensing), or in certain systems, it can be used to determine the identity of a molecule that is present within the pore based on its size (nucleic acid sequencing). Strand sequencing of nucleic acids has been described in relation to several protein based nanopores such as for mutant MspA, Clya, alpha-hemolysin and also CsgG (see WO-2016/034591).

However, protein based nanopores are very difficult to handle and design de novo. They typically exhibit pore channels with narrow lumens (less than 5 nm) that can restrict their utility in diverse bio-sensing applications. The thermodynamics of folding can be complicated and result in misfolded proteins that do not insert into and bridge the membrane. To be useful as a sensor for folded proteins or other large biomolecules, suitable membrane channels formed by a nanopore should meet certain criteria, namely:
1) the channel lumen should be at least about 5 nm wide to accommodate the large biomolecule molecules inside the channel lumen; binding of the large biomolecule within the channel results in higher read-out sensitivity than when analytes bind at the pore entrance;
2) the pores should be structurally defined to attain a constant base level in the electrical read-out (i.e. reduce background noise); and
3) the pore dimensions should be easily tunable to adapt them to different biomolecule sizes.

To date none of the existing biological or engineered pores fulfil all of these criteria.

To address the limitations of protein-based nanopores researchers have sought to use nucleic acid-based systems. Pores composed of nucleic acid duplexes, in particular DNA duplexes, have been shown to be capable of forming stable nanopores of tunable internal width (Burns J. R., et al. Angew. Chem. Int. Ed. 52, 12069-12072 (2013); and Seifert A., Göpfrich K., Burns J. R., Fertig N., Keyser U. F., Howorka S. ACS Nano 9, 1117-1126 (2015)). The modular construction principle for DNA nanopores has enabled customized pore diameter (Göpfrich et al, Nano. Lett., 15 (5), 3134-3138 (2015); WO 2013/083983) and installation of a controllable gate to regulate transport (Burns J. R., Seifert A., Fertig N., Howorka S. A., Nat. Nanotechnol. 11, 152-156 (2016)).

Membrane spanning nucleic acid nanopores are typically comprised of a structural core of a plurality of interlinked DNA duplexes that enclose a hollow channel, open at both ends. The technique of DNA origami can be used to create a stem that penetrates and spans a lipid membrane, whilst a barrel-shaped cap adheres to one side of the membrane and extends outwardly to form a funnel (Langecker et al., Science, November 16; 338 (6109): 932-936 (2012)). In this way the nucleic acid nanopore mimics the configuration of analogous protein nanopores. However, a problem associated with such configurations for both protein and nucleic acid nanopores is that the molecules, such as analytes, can be subject to non-specific binding interactions with the walls defining the internal surface of the lumen or the entry or exit apertures of the nanopore. Such non-specific binding can lead to blockage of the channel of the pore, thereby reducing the efficiency, signal and working life of devices and sensors that comprise such pores.

A further consideration with nucleic acid nanopores that are comprised of a plurality of bundled duplexes is that they can be prone to ion leakage across the pore resulting in variations in measured current. Variation of this kind is undesirable and leads to a reduction in accuracy and fidelity of signal, with significantly increased background noise. Without wishing to be bound by theory, it is considered that the variations are due to loss of structural integrity in the nucleic acid duplex bundles which are subjected to planar membrane forces causing the bundles to separate briefly or become misaligned allowing current to flow through the gaps that are formed.

It is an object of the present invention to overcome or, at least, ameliorate the problems that exist in the prior art. These and other uses, features and advantages of the invention should be apparent to those skilled in the art from the teachings provided herein.

### SUMMARY OF THE INVENTION

In a first aspect the invention provides for a membrane-spanning nanopore, the nanopore comprising:
a. one or more polynucleotide strands that provide a scaffold component; and
b. a plurality of polynucleotide strands that provide a plurality of staple components;
wherein each of the plurality of staple components hybridise to the scaffold component;
wherein the orientation of a major portion of at least one polynucleotide strand comprised within the scaffold component is substantially parallel to a planar surface of a membrane as well as embedded within and substantially coplanar with the membrane;
wherein the nanopore defines a channel that is suitable for perforating the membrane;
the channel having a longitudinal axis extending along the centre thereof and a minimum internal dimension perpendicular to the longitudinal axis of at least about 3 nm.

In a specific embodiment of the invention either or both of the one or more polynucleotide strands that provide a scaffold component and at least one of the plurality of staple components further comprise at least one hydrophobic anchor that facilitates insertion of the nanopore into the membrane.

In another embodiment, the channel has a shape perpendicular to the longitudinal axis selected from the group consisting of: annular; elliptical; and polygonal

In a specific embodiment of the first aspect of the membrane-spanning nanopore of the first aspect of the invention, the nanopore comprises:
a. one or more polynucleotide strands that provide a scaffold component; and
b. a plurality of polynucleotide strands that provide a plurality of staple components;
wherein each of the plurality of staple components hybridise to the scaffold component;
wherein the orientation of a major portion of a polynucleotide strand comprised within the scaffold component is substantially parallel to a planar surface of a membrane as well as embedded within and substantially coplanar with the membrane, such that the nanopore thereby defines a channel that perforates the membrane, the central channel having a lumen that has a minimum internal width of at least about 3 nm.

In an embodiment, the membrane-spanning nanopore of the invention comprises one or more modules. Suitably, the nanopore further comprises sub-modules connected between the one or more modules. In embodiments, the or each module are the same such that the nanopore has rotational symmetry about the longitudinal axis of the channel. In further embodiments, the or each module is connected to at least one other module. Suitably, the connection between modules is provided by structures selected from the group consisting of: a staple strand or portion thereof of one of the modules; a scaffold strand or portion thereof of one of the modules; a sub-module; one or more polynucleotide strands that provide a spacer component.

In embodiments, the scaffold component of the membrane-spanning nanopore comprises at least a second polynucleotide strand, optionally at least second and third or more scaffold polynucleotide strands. Suitably, the polynucleotide strand of the one or more polynucleotide strands comprised within the scaffold component, and/or the plurality of polynucleotide strands comprised within the staple component, and/or the spacer component, when present, comprises DNA. Typically, the assembly of the nanopores of the invention and/or components thereof is via DNA origami techniques. In a particular embodiment of the invention the one or more scaffold components, assume a stacked ring configuration that is in a coplanar orientation with respect to the membrane into which the nanopore may be embedded. In an alternative embodiment, the one or more scaffold components, assume a side-by-side configuration that, each scaffold component in a coplanar orientation with respect to the membrane into which the nanopore may be embedded.

According to specific embodiments of the invention the at least one hydrophobic anchor is comprised of a hydrophobic portion of a polynucleotide strand comprised within the scaffold component. Alternatively or additionally, the at least one hydrophobic anchor may be comprised of at least one of the plurality of polynucleotide strands comprised within the staple component which includes a hydrophobic portion. Optionally, substantially all of at least one of the staple polynucleotide strands is hydrophobic and thereby defines a hydrophobic anchor.

In a specific embodiment the at least one hydrophobic anchor comprises at least one, suitably a plurality of hydrophobic anchor molecules. Suitably, the plurality of hydrophobic anchor molecules are:
a. attached to and spaced substantially equidistantly about the periphery of the nanopore, and wherein the plurality of hydrophobic anchor molecules are orientated radially outwardly from the longitudinal axis of the channel of the nanopore; and/or
b. attached to a membrane-facing side of the nanopore or a portion thereof such that the plurality of hydrophobic anchor molecules are orientated to interact with and/or extend into the membrane once inserted.

In a further embodiment of the invention, the nanopore comprises at least three, optionally four, suitably at least five hydrophobic anchor molecules, and optionally six or more hydrophobic anchor molecules.

In embodiments of the invention the at least one hydrophobic anchor molecule is selected from: a lipid; and a porphyrin. Typically, when the anchor comprises a lipid, the lipid is selected from the group consisting of: sterols; alkylated phenols; flavones; saturated and unsaturated fatty acids; and synthetic lipid molecules (including dodecyl-beta-D-glucoside). In specific embodiments of the invention:
the sterols are selected from the group consisting of: cholesterol; derivatives of cholesterol; phytosterol; ergosterol; and bile acid;
the alkylated phenols are selected from the group consisting of: methylated phenols;
and tocopherols;
the flavones are selected from the group consisting of: flavanone containing compounds; and 6-hydroxyflavone;
the saturated and unsaturated fatty acids are selected from the group consisting of: derivatives of lauric acid; oleic acid; linoleic acid; and palmitic acids; and/or
the synthetic lipid molecule is dodecyl-beta-D-glucoside.

According to specific embodiments of the invention the nanopore is in the form of a polygon selected from the group consisting of: a triangle; a square; a quadrilateral; a pentagon; a hexagon; a heptagon; and an octagon. It is typical, but not mandatory, that the minimum internal width of the lumen defined by the channel of the nanopore is greater than about 10 nm and less than about 20 nm.

In particular embodiments of the invention the scaffold component comprises at least one polynucleotide strand is comprised of a polynucleotide having the sequence selected from the group consisting of: SEQ ID NO: 1; SEQ ID NO: 2; SEQ ID NO: 13; and SEQ ID NO: 14. Optionally, the staple components are comprised of a plurality of polynucleotide strands comprising at least one polynucleotide having a sequence selected from the group consisting of: SEQ ID NOs: 3 to 12; SEQ ID NOs: 15 to 56; SEQ ID NOs: 65 to 94; SEQ ID NOs: 105 to 140; and SEQ ID NOs: 153 to 268.

In a specific embodiment of the invention, the membrane-spanning nanopore further comprises at least one linker, wherein at least a portion of the linker is located on or proximate to a surface of the channel, when the nanopore has assumed its three-dimensional configuration. Suitably, the linker is attached to an analyte binding molecule, and wherein the analyte binding molecule is thereby located within or proximate to the channel. Suitably, the linker is a polynucleotide strand. Suitably, the linker is attached to the analyte binding molecule via a covalent linkage, electrostatic interaction or via hydrogen bonding. In specific embodiments, the analyte binding molecule comprises a molecule selected from the group consisting of: an antibody, or an antigen binding fragment thereof, including a Fab fragment; a peptide aptamer; a microprotein; an affimer; and a nucleic acid aptamer. Optionally the linker is attached to a biomolecule that possesses a catalytic activity, such as an enzyme, for example a polymerase; a helicase; a gyrase; and a telomerase. In exemplary embodiments of the invention, the biomolecule is selected from an analyte binding polypeptide or nucleic acid.

A second aspect of the invention provides a membrane-spanning nanopore, wherein the nanopore is configured to form an annular or polygonal structure that is located within a membrane, the nanopore comprising:
i. at least one scaffold polynucleotide strand; and
ii. a plurality of staple polynucleotide strands;
wherein each of the plurality of staple polynucleotide strands hybridises to the at least one scaffold polynucleotide strand to form a three-dimensional configuration of the membrane-spanning nanopore,
wherein either or both of the at least one scaffold polynucleotide strand and the plurality of staple polynucleotide strands further comprises a plurality of hydrophobic anchor molecules,
and wherein the membrane-spanning nanopore is positioned such that the orientation of a majority of the at least one scaffold polynucleotide strand is substantially parallel to the planar surface of the membrane and thereby defines a central channel with a minimum internal width of at least about 4 nm.

A third aspect of the invention provides a membrane, suitably a membrane comprising a hydrophobic bilayer or amphiphilic layer, into which is inserted at least one membrane-spanning nanopore as described in any aspect or embodiment exemplified herein. Typically the membrane comprises a semi-fluid membrane formed of polymers or a solid state membrane. Suitably, the semi-fluid membrane is composed of amphiphilic synthetic block copolymers, suitably selected from hydrophilic copolymer blocks and hydrophobic copolymer blocks. In an embodiment of the invention the hydrophilic copolymer blocks are selected from the group consisting of: poly(ethylene glycol) (PEG/PEO); and poly(2-methyloxazoline); and the hydrophobic copolymer blocks are selected from the group consisting of: polydimethylsiloxane (PDMS); poly(caprolactone (PCL); poly(lactide) (PLA); and poly(methyl methacrylate) (PMMA). In an alternative embodiment, the polymer membrane is composed of the amphiphilic block copolymer poly 2-(methacryloyloxy)ethyl phosphorylcholine-b-disisopropylamino)ethyl methacrylate (PMPC-b-PDPA). Optionally the membrane is, or comprises, a biological lipid bilayer membrane.

In a specific embodiment of the invention the membrane is in the form of a vesicle, a micelle, a planar membrane or a droplet.

In another embodiment, the membrane is a solid state membrane formed of a material selected from the group consisting of: Group II-IV and III-V oxides and nitrides, solid state organic and inorganic polymers, plastics, elastomers, and glasses.

A fourth and fifth aspect of the invention provides a biological sensor, wherein the biological sensor comprises at least one membrane as described herein and also apparatus for electrical or fluorescence measurement respectively. A further embodiment of the invention provides for a biological sensor that comprises a plurality of membrane-spanning nanopores comprised within one or more membranes.

A sixth aspect of the invention provides for a sensing device comprising one or more biological sensors as described herein. The sensing device may serve as a diagnostic or prognostic apparatus. The sensing device may comprise electrodes arranged on each side of the membrane in order to measure an ion current through an aperture/membrane perforation under a potential difference. The electrodes may be connected to an electrical circuit which includes a control circuit arranged to supply a voltage to the electrodes and a measurement circuit arranged to measure the ion flow. A common electrode may be provided to measure ion flow through the apertures between the common electrode and electrodes provided on the opposite side of the membrane.

A seventh aspect of the invention provides a method for molecular sensing comprising:
I. providing a membrane as described herein;
II. contacting the nanopore with an analyte and establishing a flow of ions through the at least one nanopore or an electron flow across the nanopore; and
III. measuring an electrical signal across the nanopore,
wherein the molecular sensing comprises analyte detection or characterisation, wherein a change in electrical measurement is indicative of the analyte.

In an embodiment of the present invention, the electrical measurement is selected from: a current measurement; an impedance measurement; a tunnelling measurement; and a field effect transistor (FET) measurement. Optionally, the electrical measurement comprises determining a flow of ions from a first side of the membrane to a second side of the membrane. Optionally, the method described comprises after step (III) the further step of determining the presence of the analyte by a change in ion flow or electron flow through or across the one or more membrane-spanning nanopores compared to the ion flow or electron flow through or across the one or more membrane-spanning nanopores when the analyte is absent.

In a eighth aspect of the invention, a method for molecular gating is provided, the method comprising:
a) Providing the membrane according to the third aspect of the invention;
b) Providing at least one biomolecule with a diameter of less than the minimum internal width of a channel in the nanopore; and
c) Incubating until the at least one biomolecule has passed through the nanopore.

In an embodiment of the eighth aspect of the invention, when at least one biomolecule has passed through the nanopore, it is subjected to a physical change that prevents it returning through the nanopore. In further embodiments, the at least one biomolecule is a globular protein, a polynucleotide-protein construct, a labelled polynucleotide or a labelled protein.

It will be appreciated that the invention may be defined by further combinations of several of the features disclosed herein but which are not explicitly recited above.

### BRIEF DESCRIPTION OF THE DRAWINGS

The invention is illustrated by the accompanying drawings in which:

FIG. 1 (a) is a representation of a nucleic acid nanopore structure according to one embodiment of the invention showing a stacked ring-like configuration but omitting the presence of separate lipid anchors in top (plan) view showing the channel that defines a lumen of internal width of 10 nm and in side view; (b) is a representation of a nucleic acid nanopore structure according to one embodiment of the invention with a linker group and analyte binding agent (biotin) tethered within the channel defined by the pore; (c) shows a representation of a nucleic acid nanopore structure according to one embodiment of the invention showing the ring-like configuration with the presence of six membrane anchors extending radially outwardly in top (plan) and oblique view-denoted as r6c; and (d) is a representation of ring nanopores according to the embodiment of (a) which are inserted into a lipid membrane, thereby permitting the passage of analytes across the membrane via the channel of the nanopore.

FIG. 2 shows a 2D DNA map of an r6c ring nanopore according to one embodiment of the present invention (the sequences of which are set out in Example 1). The scaffold strand is shown in medium grey and is a longer continuous strand, whereas the staple strands are in dark grey and are much shorter in length. 5′ and 3′ termini of DNA strands are represented as a squares and triangles, respectively. The circles around the 3′ termini along the central axis indicate placement of the anchor molecules. The circle marked (A) identify the 3′ terminus where a linker/analyte interacting molecule/functionalising molecule is attached.

FIG. 3 (a) shows a photograph of an electrophoresis gel showing the results of an experiment in which: M-100 bp DNA ladder, lane 1-scaffold only, lane 2-linear structure (L, linearized ring), lane 3-ring structure (roc, without cholesterol); lane 4 and 5-ring nanopore structure bearing four and six cholesterol tags (r4c and r6c), respectively; lane 6-cholesterol-DNA only; and (b) representative TEM images of the roc ring (left) and a ring nanopore structure incubated with SUVs-(scale bar=20 nm).

FIG. 4 shows confocal fluorescence images of GUV vesicles incubated with fluorophore-labelled DNA ring r6c. From left to right: Green channel-fluorophore-labelled lipid, Red channel-DNA ring, Merged image of the two channels. Scalebar=20 μm.

FIG. 5 shows a graph depicting time-dependent releasing of 6-Carboxyfluorescein (6-CF) from SUVs, after adding nanopore ring structures of one embodiment of the invention without or with various number of membrane anchors (cholesterol tags); roc-ring structure without cholesterol tags; r2c, r4c and r6c-ring structures with two, four and six cholesterol tags, respectively; L5c-linear structure (control) with five cholesterol tags.) Release of 6-CF from SUVs is only observed for nanopore rings with 4 and 6 cholesterols, but not for the linearized ring with 5 cholesterol lipid anchors.

FIG. 6 shows a time-series of confocal images of dye influx mediated by nanopore rings (r6c) into GUVs. FITC-dextran (Left, hydrodynamic diameter of 15 nm) and Atto633 (Right, h.d diameter of 1 nm) were measured simultaneously.

FIG. 7 shows a graph of the results shown in FIG. 6 based upon fluorescence intensity measurements of the two dyes in the GUV lumen.

FIG. 8 shows schematic representations of the design of polygonal DNA nanopores and their exemplary TEM images. All scalebars are 20 nm in length.

FIG. 9 shows (A) top view and (B) bottom view of the 10 nm wide DNA square-shaped pore structure binding with cholesterol strands (medium grey cone); (C) schematic representation of a membrane-bound 10 nm DNA nanopore; (D) 1.2% agarose gel analysis of the pore structure and its incubation with cholesterol strands and DPhPC SUVs: M-1 kb DNA ladder; lane 1-4: M13 scaffold, DNA pore, DNA pore bound with cholesterol strands, after incubating of the cholesterol-bound pore with DPhPC SUVs; (E)-(F) confocal images indicating the binding of DNA pore on the GUV [(E)-POPC with 0.5% Bodipy-C12-HPC; (F)-Atto647N-labelled DNA structure; scalebar=20 μm]; TEM analysis of the 20 nm wide nanopore (G) and membrane-bound nanopore (H). Scalebars in G and H are 50 nm.

FIG. 10 shows a comparison of single-channel current recordings of different polygonal DNA nanopores inserted into planar lipid bilayers. Example trace and IV curve of A-10 nm×10 nm triangle DNA nanopore, B-10 nm×10 nm square DNA nanopore and C-20 nm×20 nm square DNA nanopore. Experiments were carried out using membranes formed of DPhPC, and electrophysiological buffers composed of 1M KCl, 10 mM HEPES PH 8.

FIG. 11 shows a two-dimensional DNA map of a 10 nm frame of a triangular nanopore in accordance with an embodiment of the invention. Scaffold strands are depicted in light grey; staple strands are depicted in medium grey.

FIG. 12 shows a two-dimensional DNA map of a 10 nm frame of a square nanopore in accordance with an embodiment of the invention. Scaffold strands are depicted in light grey; staple strands are depicted in medium grey; and spacer strands are depicted in dark grey.

### DETAILED DESCRIPTION OF THE INVENTION

Prior to setting forth the invention, a number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘membrane’ as used herein is an enclosing or separating selectively-permeable boundary, partition, barrier or film. The membrane has two sides or surfaces which may be named the cis and trans side respectively. The membrane is thin (i.e. has a thickness substantially less than its width and length) allowing it to be spanned by the nanopore. In the context of the present invention, the membrane thickness is the typically in the nanometre (10-9 metre) range. The arrangement of the membrane is not limited and may assume any form, for example, a liposome, and a vesicle or as a planar or a non-planar sheet. Specific examples of membranes useful in the present invention include lipid bilayers, polymeric films, or solid state substrates.

The term ‘solid state membrane’ or ‘solid state substrate’ as used herein refers to a membrane formed from a solid state substance—i.e. not a semi-fluid membrane—in which one or more apertures are provided. One or more nanopores may be positioned within the respective one or more apertures disclosed for example in U.S. Pat. No. 8,828,211, hereby incorporated by reference. The solid state membrane may comprise either or both of organic and inorganic materials, including, but not limited to, microelectronic materials, whether electrically conducting, electrically semiconducting, or electrically insulating, including materials such as II-IV and III-V materials, oxides and nitrides, such as silicon nitride, Al₂O₃, and SiO₂, Si, MoS₂, solid state organic and inorganic polymers such as polyamide, plastics such as Teflon®, or elastomers such as two-component addition-cure silicone rubber, and glasses. A membrane may be formed from monatomic layers, such as graphene, or layers that are only a few atoms thick such as those disclosed in U.S. Pat. No. 8,698,481, and U.S. Patent Application Publication 2014/174927, both hereby incorporated by reference. More than one layer of material can be included, such as more than one graphene layer, as disclosed in US Patent Application Publication 2013/309776, incorporated herein by reference. Suitable silicon nitride membranes are disclosed in U.S. Pat. No. 6,627,067, and the membrane may be chemically functionalized, such as disclosed in U.S. Patent Application Publication 2011/053284, both hereby incorporated by reference. Such a structure is disclosed for example in U.S. Pat. No. 8,828,211, hereby incorporated by reference. The internal walls of the apertures may be coated with a functionalised coating, such as disclosed in published application WO 2009/020682. The one or more apertures may be hydrophobic or provided with a hydrophobic coating to assist the provision of the one or more nanopores in the respective one or more apertures. Suitable methods for providing apertures in solid state substrates are disclosed in published applications WO 03003446 and WO 2016/187519.

The term ‘modular’ as used herein refers to the use of one or more units, or modules, to design or construct a whole or part of a larger system. In the context of the present invention it refers to the use of individual modules, sub-units or building blocks to construct a nanopore. The modules may be each the same or the modules may be different. To form the nanopore, the individual modules may be connected or inter-linked to one or more other modules. The means of connection between modules may be by chemical or physical means, such as covalent or non-covalent chemical bonding or by electrostatic or other attractive forces. Alternatively, or in addition, the means of connection may be via an additional module, bracing member, portion or linkage. The modular design of a nanopore may comprise a frame or framework of modules, and additional, typically smaller, sub-modules that connect, or support the frame, acting as struts or bracing members. The modules span the membrane to enable formation the channel of the nanopore; the sub-modules do not generally span the membrane and are intended only as structural support in the nanopore. The design of the modules and sub-modules, or how they connect, may be chosen to support and strengthen the formed channel of the nanopore such that it maintains its shape and conformational integrity when inserted in a membrane. The modules or sub-modules may be formed of nucleic acids, typically DNA. Each individual unit may be assembled by DNA origami techniques described elsewhere herein using suitably selected scaffold and staple strands. The individual modules may be joined by further DNA strands, or by direct bonding between the DNA strands of the individual modules. In some cases, the individual modules are arranged radially around a central axis, and thereby form, a channel. In other cases, the individual modules may be stacked on top of one another in a direction perpendicular to the plane of the membrane to form a tower or pile. In some instances, the tower or pile may be shaped in cross section such that the tower or pile forms a channel of sufficient length to span a membrane within which the nanopore is to be inserted. Any combination or arrangement of modules is envisaged such that they can form a channel that spans a membrane.

The term ‘nucleic acid’ as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, and are typically manufactured synthetically, but may also be isolated from natural sources. Nucleic acids may further include modified DNA or RNA, for example DNA or RNA that has been methylated or that has been subject to chemical modification, for example 5′-capping with 7-methylguanosine, 3′-processing such as cleavage and polyadenylation, and splicing, or labelling with fluorophores or other compounds. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Hence, where the terms ‘DNA’ and ‘RNA’ are used herein it should be understood that these terms are not limited to only include naturally occurring nucleotides. Sizes of nucleic acids, also referred to herein as ‘polynucleotides’ are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 100 nucleotides in length are typically called ‘oligonucleotides’.

As used herein, the terms ‘3” (‘3 prime’) and ‘5” (‘5 prime’) take their usual meanings in the art, i.e. to distinguish the ends of polynucleotides. A polynucleotide has a 5′ and a 3′ end and polynucleotide sequences are conventionally written in a 5′ to 3′ direction. The term ‘complements of a polynucleotide molecule’ denotes a polynucleotide molecule having a complementary base sequence and reverse orientation as compared to a reference sequence.

The term ‘duplex’ is used herein refers to double-stranded DNA, meaning that the nucleotides of two complimentary DNA sequences have bonded together and then coiled to form a double helix. According to the present invention, homology to the nucleic acid sequences described herein is not limited simply to 100% sequence identity. Many nucleic acid sequences can demonstrate biochemical equivalence to each other despite having apparently low sequence identity. In the present invention homologous nucleic acid sequences are considered to be those that will hybridise to each other under conditions of low stringency (Sambrook J. et al, Molecular Cloning: a Laboratory Manual, Cold Spring Harbor Press, Cold Spring Harbor, NY).

As used herein, the term ‘nanostructure’ refers to a predesigned two or three dimensional molecular structure typically comprised from a biopolymer, suitably a naturally or non-naturally occurring nucleic acid, which structure has at least one dimension or an aspect of its geometry that is within the nanoscale (i.e. 10-9 metres). Nanoscale structures suitably have dimensions or geometry of less than around 100 nm, typically less than 50 nm, and most suitably less than 20 nm. Nanoscale structures suitably possess dimensions or geometry greater than around 0.1 nm, typically greater than around 1 nm, and optionally greater than around 2 nm. Assembly of nucleic acid nanostructures may occur spontaneously in solution, such as by heating and cooling a mixture of DNA strands of preselected sequences, or may require presence of additional co-factors including, but not limited to, nucleic acid scaffolds, nucleic acid aptamers, nucleic acid staples, co-enzymes, and molecular chaperones. Where desired nanostructures result from one or more predesigned spontaneously self-folding nucleic acid molecules, such as DNA, this is typically referred to as nucleic acid ‘origami’. Rational design and folding of DNA to create two dimensional or three dimensional nanoscale structures and shapes is known in the art (e.g. Rothemund (2006) Nature 440, 297-302). In the classical scaffold-and-staple approach, one or more long biogenic scaffold strand component(s) is folded into a defined DNA nanostructure with a staple component consisting of shorter synthetic staple oligonucleotides. Classical DNA nanostructures are formed of bundles of parallel aligned DNA duplexes that are arranged into polygons that enclose a channel and puncture a membrane bilayer. However, a challenge with nucleic acid nanostructures remains that the strong net negative charge of the phosphodiester background hinders insertion into amphipathic and hydrophobic planar membranes. As a result, this has often favoured their use in solid state contexts, as nanofunnels attached to or sited within a nanoscale aperture in a substrate. A problem associated with such arrangements, however, is that they can often exhibit high levels of ionic leakage in sensor applications due to poor fit between the DNA duplex and the nanoscale aperture. Ionic leakage is much reduced when nanopores are embedded within semi-fluid membranes which surround the pore.

The nucleic acid sequences that form the nanostructures will typically be manufactured synthetically, although they may also be obtained by conventional recombinant nucleic acid techniques. DNA constructs comprising the required sequences may be comprised within vectors grown within a microbial host organism (such as E. coli). This would allow for large quantities of the DNA to be prepared within a bioreactor and then harvested using conventional techniques. The vectors may be isolated, purified to remove extraneous material, with the desired DNA sequences excised by restriction endonucleases and isolated, such as by using chromatographic or electrophoretic separation.

The term ‘amino acid’ in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A ‘polypeptide’ is a polymer of amino acid residues joined by peptide bonds, whether produced naturally or in vitro by synthetic means. Polypeptides of less than around 12 amino acid residues in length are typically referred to as ‘peptides’ and those between about 12 and about 30 amino acid residues in length may be referred to as ‘oligopeptides’. The term ‘polypeptide’ as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term ‘protein’ is used herein to refer to a macromolecule comprising one or more polypeptide chains.

The term ‘folded protein’ as used herein refers to a protein that has acquired some three-dimensional shape after translation of the polypeptide chain from which it is formed (the primary structure). The term may refer to the secondary structure of the protein which is typically the first stage of the folding process where local three-dimensional structures are formed, for example, alpha helices or beta sheets. The term may more typically refer to the tertiary structure of a protein where the secondary structures of the protein have folded to stabilise the structure through hydrophobic or covalent interactions. The term also encompasses proteins having a quaternary structure where one or more protein subunits are assembled. As appropriate, the folded protein may also be termed the ‘native’ protein structure, and may be the form of the protein that exhibits its biological function.

The term ‘interior width’ when used herein refers to the straight distance spanning the interior of the channel (e.g. the lumen) from an interior face of one wall to an interior face of an opposing wall in a plane perpendicular to the longitudinal axis of the channel. The interior width of the channel may be constant along its longitudinal axis or it may vary due to the presence of one or more constrictions. The ‘minimum interior width’ is the minimum interior width along the longitudinal axis of the channel between an entrance and an exit of the channel. The minimum interior width of a channel defines the maximum size of an object, such as an analyte, that may pass through the channel.

As used herein the term ‘hydrophobic’ refers to a molecule having apolar character including organic molecules and polymers. Examples are saturated or unsaturated hydrocarbons. The molecule may have amphipathic properties.

As used herein, the term ‘hydrophobically-modified’ relates to the modification (joining, bonding or otherwise linking) of a polynucleotide strand with one or more hydrophobic moieties. A ‘hydrophobic moiety’ as defined herein is a hydrophobic organic molecule. The hydrophobic moiety may be any moiety comprising non-polar or low polarity aliphatic, aliphatic-aromatic or aromatic chains. Suitably, the hydrophobic moieties utilised in the present invention encompass molecules such as long chain carbocyclic molecules, polymers, block co-polymers, and lipids. The term ‘lipids’ as defined herein relates to fatty acids and their derivatives (including tri-, di-, monoglycerides, and phospholipids), as well as sterol-containing metabolites such as cholesterol. The hydrophobic moieties comprised within the embodiments of the present invention are capable of forming non-covalent attractive interactions with phospholipid bilayers, such as the lipid-based membranes of cells and act as membrane anchors for the nanopore. According to certain embodiments of the present invention suitable hydrophobic moieties, such as lipid molecules, possessing membrane anchoring properties may include sterols (including cholesterol, derivatives of cholesterol, phytosterol, ergosterol and bile acid), alkylated phenols (including methylated phenols and tocopherols), flavones (including flavanone containing compounds such as 6-hydroxyflavone), saturated and unsaturated fatty acids (including derivatives such as lauric, oleic, linoleic and palmitic acids), and synthetic lipid molecules (including dodecyl-beta-D-glucoside). The anchors for the polymer membrane may be the same as for lipid bilayers or they may be different. The specific hydrophobic moiety anchor may be selected based on the binding performance of the membrane chosen.

In an alternative embodiment of the invention, the hydrophobic modification is comprised within one or more synthetic nucleic acids (XNAs) incorporated into the nanopore structure itself.

The inventors have identified a structurally-defined wide-channel low profile organic nanopore. The structure of the nanopore renders it suitable for a number of uses in which a nanopore is positioned in a membrane that partitions two solutions. One such use is biomolecule sensing applications, in particular protein sensing applications where the folded protein may pass, bind or lodge within the pore. Modified versions of the nanopore may serve to further enhance the suitability of the channel for a particular folded protein or other biomolecule. Biomolecule sensing may be enhanced by the installation of a molecular receptor, binding molecule or enzyme within the pore lumen or channel.

The ability to create nanopores with controlled pore sizes and insert them into natural or synthetic membranes allows the construction of customised selectively-permeable membranes where control over the lumen dimensions, and optionally, other features of the pores enables control over which biomolecules are able to pass through these membranes. Of particular utility in the present invention is the increased control over internal width/size of the nanopores of the present invention that allows large biomolecules, including large globular proteins, to pass through such membranes. This has potential utility in medicine alongside other uses in the field of biology. For example, there are several large i.e. 10-30 nm diameter membrane pores that are either produced by immune cells to kill bacteria, such as in the ‘complement system’ (a part of the innate immune system that enhances (complements) the ability of antibodies and phagocytic cells to clear microbes and damaged cells from an organism, promotes inflammation, and attacks the pathogen's plasma membrane).

As an example, vesicles or microscale compartments formed of natural or synthetic polymers comprising nanopores according to the present invention may be used as nanoreactors. It is envisaged that substrate proteins or biomolecules may enter the vesicle interior through the nanopore where encapsulated enzymes in the interior of the vesicle enable a desired reaction to take place within the vesicle. As an example, encapsulated protease enzymes, such as trypsin may be retained inside a vesicle where the relatively large and controllable size of the nanopores could allow some substrate proteins but not others to enter for degradation, thereby protecting particular proteins from indiscriminate digestion. Similarly, control over the release of cargo contained within similar vesicles, such as protein or peptide based pharmaceutical agents would be possible with membranes with nanopores which allow passage of the cargo. Nanoreactors as described herein can be used in microfluidic biosynthetic processes and as part of lab-on-a-chip devices.

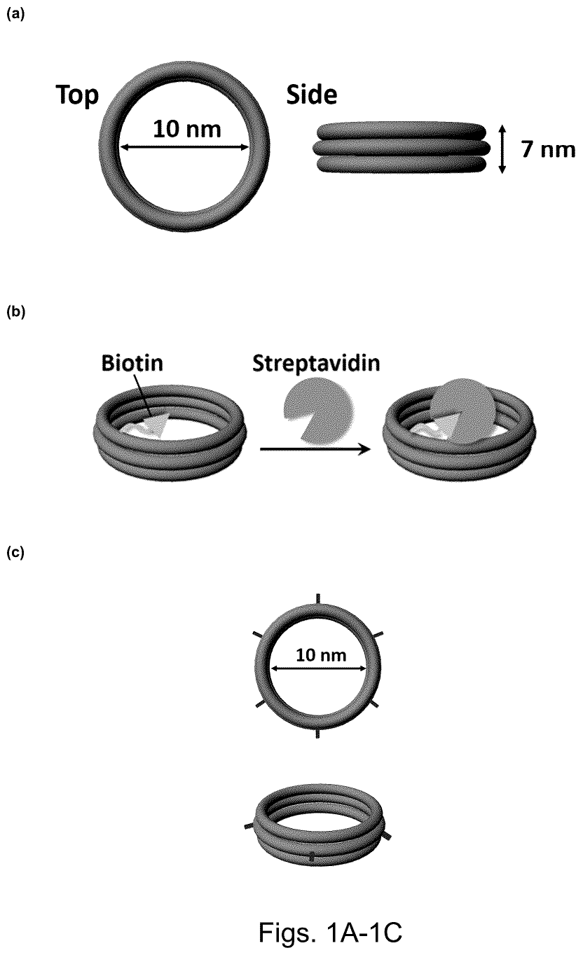

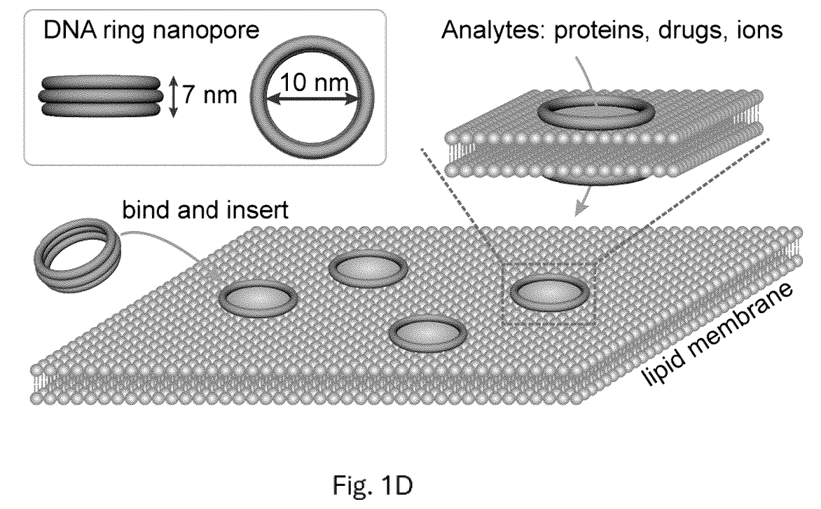

The nanopore according to an embodiment of the present invention a membrane-spanning nanostructure that is embedded within and, at least a portion thereof, is oriented substantially coplanar to a membrane. In an embodiment of the invention, the nanopore is located in a membrane in a manner akin to a grommet or eyelet mounted in a planar or curved sheet material. Hence, in a specific embodiment of the invention the nucleic acid nanostructure of the invention is defined as a nanoscale grommet or eyelet, this is irrespective of the shape of the channel(s) which may be circular or polygonal. A visual representation of one such arrangement is set out in FIG. 1 (d), where an embodiment showing a ring nanopore is shown embedded within a planar lipid membrane. According to this embodiment of the invention it is apparent that a majority of the nanostructure of the nanopore is embedded within the membrane compared to the proportion of the nanostructure extending outside of the membrane. This is in direct contrast to many previous nucleic acid nanopores.

Suitably the nanopores of the invention comprise one or more polynucleotide strands that provide a functional scaffold component, wherein the polynucleotide strands comprised within the scaffold component include a polynucleotide backbone; and a plurality of polynucleotide strands that provide a plurality of functional staple components. The scaffold strand(s) cooperate with and hybridise to the plurality of staple polynucleotide strands—e.g. via appropriate Watson-Crick base pairing hybridisation—in order to form a three-dimensional configuration of the nanopore. It is desirable that the nanopore assembles into an annular, elliptical or polygonal structure that is able to be embedded within a membrane such that a majority of the 5′ to 3′ orientation of the staple component(s) is coplanar with the membrane.

Suitably, the nanopores of the present invention are assembled via the ‘scaffold-and-staple’ approach. In this important route to DNA nanostructures, DNA is utilized as a building material in order to make nanoscale three dimensional shapes. Assembly of these complex nanostructures from a plurality of un-hybridized linear molecules is typically referred to as ‘DNA origami’.

The DNA origami process generally involves the folding of the one or more elongate, ‘scaffold’ DNA strands into a particular shape using a plurality of rationally designed ‘staple’ DNA strands. The scaffold strand can have any sufficiently non-repetitive sequence. The sequences of the staple strands are designed such that they hybridize to particular defined portions of the scaffold strands and, in doing so, these two components cooperate force the scaffold strands to assume a particular structural configuration. Methods useful in the making of DNA origami structures can be found, for example, in Rothemund, P. W., Nature 440:297-302 (2006); Douglas et al, Nature 459:414-418 (2009); Dietz et al, Science 325:725-730 (2009); and U.S. Pat. App. Pub. Nos. 2007/0117109, 2008/0287668, 2010/0069621 and 2010/0216978, each of which is incorporated by reference in its entirety. Staple sequence design can be facilitated using, for example, CaDNAno software, available at http://www.cadnano.org.

In embodiments of the invention the staple and/or scaffold components further comprise a plurality of hydrophobic membrane anchor molecules that are attached thereto. The hydrophobic anchors (or portions of the sequence) facilitate insertion of the nanopore into a curved or planar membrane such that the orientation of a major portion of the first scaffold polynucleotide strand is substantially parallel to the surface of the membrane and wherein the first scaffold polynucleotide strand is embedded within and is substantially coplanar with the membrane. In referring to ‘a major portion’ of a scaffold polynucleotide stand it is meant that substantially more than 50%, suitably more than 60%, even greater than 70%, and as much as 90%, of the total length of that strand is orientated so that it is substantially coplanar with the membrane. In a specific embodiment the entire length of the scaffold strand, excepting crossovers and/or Holliday junctions necessary to impart three dimensional structure on a resultant nanopore, is orientated so that it is substantially coplanar with the membrane. It will be appreciated that DNA origami techniques allow for variations of the embodied structures that, nevertheless, fall within the overall design constraints of the recited nanostructures of the present invention. By way of example, the scaffold strand may be comprised of a plurality of shorter scaffold strands that, when assembled following hybridisation to appropriate staple strands, will cooperate to serve in a manner equivalent to a unitary single length scaffold strand.

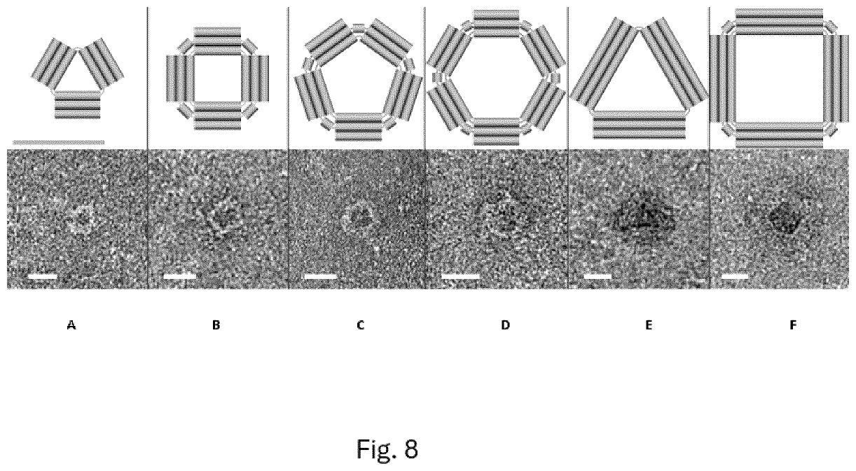

In embodiments, the nanopores of the present invention are formed or constructed from one or more modules. In embodiments, the nanopore may be formed of an arrangement of modules that forms a basic frame or framework. In embodiments, the modules of the frame are supported by additional, typically smaller, sub-modules that connect and support the structure of the frame. At least part of the module is intended to form at least part of the channel wall of the nanopore and therefore the module is designed and configured to span a membrane in which it is inserted. Sub-modules are intended for structural benefits only and therefore are not intended or configured to span the membrane. While each module of the frame, or the sub-modules, may be different, suitably, the modules of the frame are suitably substantially or completely identical units, as are the sub-modules forming the support, when present. In these embodiments, the modules and sub-modules are each formed of the same scaffold and staple DNA structure and assembled in the same way. The individual modules may be joined by DNA strands, the DNA strand either being integral with the module, or hybridised to each module. While any arrangement of the modules is contemplated, suitably, for circular or elliptical cross-section nanopores, the modules may be arranged to overlie each other to form a generally hollow stack or tower thereby forming a channel. Suitably, for nanopores having a polygonal cross-section, the modules are arranged such that they sit side by side in the plane of a membrane. A combination of the above arrangements is also contemplated. The modules may have tuneable side length (a side length in this context being defined as the longest dimension of the module parallel to the plane of the membrane), which when chosen with an appropriate final overall shape, allows for different sized and/or shaped nanopores, and different sized and/or shaped lumens within the channels of those pores to be prepared. The channels and the lumens defined thereby may be regularly or irregularly shaped. For example, the lumens defined by the channel may be a regular or irregular circle or polygon, such as a triangle, a quadrilateral (e.g. a square, a rectangle or a trapezoid), a pentagon, a hexagon, a heptagon, an octagon and so on. Alternatively, the channels may be an elongate circle (ellipse) or elongate polygon, such as rectangular, oblong or slot-shaped channels formed of 4 or more sides. Typically, the side length of the modules would be in the order of between 10 nm and 20 nm (FIG. 8). Suitably, the side length of the modules may be at least 2 nm, 3 nm, 4 nm, 5 nm, 6 nm, 7 nm, 8 nm, 9 nm or 10 nm, Suitably the side length of the modules may be at most 30 nm, 25 nm, 20 nm. The sizing of the sub-modules is determined by the spacing between the modules which is turn is determined by the shape of the pore and the size and number of modules employed. Suitably, the side length of the modules may be at least 0.5 nm, 1 nm, 1.5 nm, 2 nm, 2.5 nm, 3 nm, 3.5 nm, 4 nm or 5 nm, Suitably the side length of the modules may be at most 10 nm, 7.5 nm, or 5 nm.

As used herein, the term ‘substantially’ refers to the complete or nearly complete extent or degree of an action, characteristic, property, state, structure, item, or result. For example, an object that is ‘substantially’ coplanar with another object would mean that the object is either completely coplanar or nearly completely coplanar, perhaps varying by a few degrees of variation from complete conformity. The exact allowable degree of deviation from absolute completeness may in some cases depend on the specific context, as would be understood to the person of skill in the art. However, in general terms the nearness of conformity to the absolute will be such as to have the same overall result—e.g. functional equivalence—as if total conformity were achieved.

The three-dimensional configuration of the nanopores of the present invention defines at least one channel, suitably a single channel that spans the membrane, the channel having a lumen that has a minimum internal width of at least about 3 nm. Suitably, the nanopores of the present invention have a single channel located at least substantially centrally in the pore structure when viewed perpendicular to the plane of the membrane in which the pore is intended to reside. The channel defines the lumen that passes through the nanopore which is perpendicular to the planar axis defined by the membrane. The cross-sectional profile of the lumen parallel to the plane of the membrane may be circular, ellipsoid or polygonal and may vary in terms of the internal dimensions. Suitably, the channel has a consistent internal cross-sectional profile and size for its entire length. Suitably, the cross-sectional profile of the channel is a circle or a quadrilateral, typically a square, rectangle or trapezoid. The minimum opening of the channel in this cross-section (i.e. minimum constriction) is suitable to allow access for a folded protein or other analyte. Typically, the minimum opening is at least 3 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, or 15 nm or more. Suitably the lumen is between around 10 nm and around 20 nm in width. The maximum opening of the channel is limited only by the need to maintain structural integrity of the pore and to obtain an electrical read-out when a molecule of interest passes through. Suitably, the maximum opening of the channel (i.e. minimum constriction) is at most 200 nm, 150 nm, 100 nm, 75 nm, 50 nm, 40 nm, 30 nm, 20 nm, 18 nm, 15 nm, 12 nm, or 10 nm. Suitably, the cross-sectional area of the minimum opening of the channel (i.e. minimum constriction) is at least 5 nm², 12 nm², 15 nm², 25 nm², 35 nm², 40 nm², 45 nm² or 50 nm² or more. Suitably, the cross-sectional area of the minimum opening of the channel (i.e. minimum constriction) is at most 35 000 nm², 25 000 nm², 15 000 nm², 10 000 nm², 5 000 nm², 1500 nm², 1000 nm², 750 nm², 500 nm², 250 nm², 100 nm², 50 nm², 30 nm², 20 nm² or 15 nm², 10 nm², 7 nm² or less.

To achieve optimum performance, the variation in the channel size should be minimized from pore to pore. Even small variations in cross sectional area can lead to a significant discrepancy in ion flow through a given pore, both in the open state (devoid of any target analyte), or in the bound state (with target analyte present in, or proximate to, the channel). Such variation in ion flow leads to a lower signal to noise ratio in the electrical read-out thereby reducing the sensitivity of detection. The nanopores according to the present invention can have low variability in pore size if desired.

As mentioned, in embodiments of the present invention the nanostructures may comprise a channel that has a consistent internal profile and size (i.e. lumen width) for its entire length. However, for certain sensing applications it may be advantageous for the lumen width to vary along the length of the channel with one or more internal constrictions introduced. Such constrictions may be engineered using the same DNA origami techniques that are used in order to design the nanostructure as a whole. Alternatively, constrictions may be added post hoc by way of chemical modification or binding of a blocking molecule to effect a constriction. The introduction of one or more constrictions may affect the kinetics of ion and analyte flow through the nanopore in a similar way to the creation of a funnel.

The nanopore of the present invention may comprise two or more membrane anchors that act to attach or connect or anchor the hydrophilic DNA nanopore to the generally hydrophobic membrane (lipid bilayer or polymer). The lipid anchors are attached to the pore. Suitably attachment is via DNA oligonucleotides that carry the lipid anchor, suitably cholesterol, at the 5′ or 3′ terminus. Polynucleotides or oligonucleotides may be functionalized using a modified phosphoramidite in the strand synthesis reaction, which is easily compatible for the addition of reactive groups, such as cholesterol and lipids, or attachment groups including thiol and biotin. Enzymic modification using a terminal transferase can also be used to incorporate an oligonucleotide, which incorporates a modification such as an anchor, to the 3′ of a single stranded nucleic acid (e.g. ssDNA). These lipid modified anchor strands may hybridize via ‘adaptor’ oligonucleotides to corresponding sections of the DNA sequence forming the scaffold section of the pore. Alternatively, the lipid anchors are assembled with the pore using lipid-modified oligonucleotides that contribute as either the scaffold or staple strands. A combination of approaches to anchoring using two or more membrane anchors may also be adopted wherein anchors are incorporated into one or all of a scaffold strand, a staple strand and an adaptor oligonucleotide.

Cholesterol has been found to be a particularly suitable lipid for use as an anchor in the present invention. The use of other lipids as anchors is contemplated, although it may be expected that there is a particular preference for a particular lipid, and a given number of membrane anchors, for a given membrane.

In a particular embodiment of the invention the membrane anchors are positioned around the periphery of the nanopore (i.e. away from the channel) such that they may extend radially outwardly from the nanostructure and interact with the amphipathic membrane that surrounds and encloses the nanostructure. In an alternative embodiment, the membrane anchors are positioned on a membrane-facing surface of the nanopore such that they may extend radially outwardly from the nanostructure and interact with the amphipathic membrane that surrounds and encloses the nanostructure. Suitably the plurality of membrane anchors are positioned substantially equidistantly about the periphery of the nanostructure. In this way insertional forces may be distributed more evenly about the outer periphery of the nanopore. By way of example, where two membrane anchors are used they will be spaced about 180° from each other; where three membrane anchors are used spacing between each is about 120°; for four membrane anchors spacing is around 90°; for five membrane anchors spacing is around 72°; for six membrane anchors spacing is around 60°; and for seven spacing is around 52°. It will be appreciated that the spacing will diminish accordingly for greater number of membrane anchors.

In an alternative embodiment of the invention where the hydrophobic modification is comprised within one or more synthetic nucleic acids (XNAs) incorporated into the nanopore structure, one or more of the scaffold and/or staple strands may be fully or partially comprised of a synthetic nucleic acid analogue. Advantageously, the presence of hydrophobic synthetic nucleic acids enables the nanopore to interact and embed within a membrane. In such an embodiment the presence or one or more additional hydrophobic membrane anchor molecules may be unnecessary, with the requisite level of hydrophobic membrane anchoring capability comprised within the backbone of the nanostructure itself. Hybrid structures comprising a combination of hydrophobic synthetic nucleic acids together with lipid-based membrane anchors are also contemplated as part of the invention.

The membrane in which the nanopore of the present invention may be inserted may be of any suitable type. Depending on the intended use, the membrane may be a lipid bilayer or a polymer sheet or film. The membrane is suitably an amphiphilic layer. The amphiphilic layer may be a monolayer or a bilayer. An amphiphilic layer is a layer formed from amphiphilic molecules which have both hydrophilic and lipophilic properties. The amphiphilic molecules may be synthetic or naturally occurring. The lipophilic properties of the molecules comprising the membrane promote anchoring by lipid anchors or other hydrophobic anchoring regions of the nanopore. It is surprising that nucleic acid nanostructures of the invention are able to be inserted successfully into membranes at all given that the hydrophobic nature of the membrane leads to repulsion of the predominantly negatively charged DNA backbone. It might be expected that the nanostructures of the invention would simply form clustered aggregates on one surface of the membrane as is often the case with complex nucleic acid nanostructures that fail to insert successfully into an amphiphilic mono- or bilayer. The ability of the nanostructures to be embedded within such membranes is, therefore, surprising and most unexpected.

In a specific embodiment of the invention the amphiphilic layer may be a lipid bilayer. The lipid composition may comprise naturally-occurring lipids such as phospholipids and bipolar tetraether lipids, and/or artificial lipids. The lipids typically comprise a head group, an interfacial moiety and two hydrophobic tail groups which may be the same or different. Suitable head groups include, but are not limited to, neutral head groups, zwitterionic head groups, negatively charged head groups and positively charged headgroups. The head group or the tail group of the lipids may be chemically-modified.

It is envisaged that nanopores according to the present invention may be inserted into a membrane of a target cell or vesicle to facilitate translocation of specific folded proteins across the membrane. The specificity of the translocation to certain folded proteins may be controlled through variation of the size of the channel in the nanopore.

Proprietary and non-proprietary synthetic polymer films or sheets are widely used in ‘chip-based’ nanopore sequencing and analytical applications such as the MinION® system sold by Oxford Nanopore Technologies®; the GS FLX+® and the GS Junior® System sold by Roche®; the HiSeq®, Genome Analyzer IIx®, MiSeq® and the HiScanSQR systems sold by Illumina®; the lon PGM® System and the lon Proton System® sold by Life Technologies; the CEQ® system sold by Beckman Coulter®; and the PacBio RS® and the SMRT® system sold by Pacific Biosciences®. The ability of nanopores to insert successfully into polymer membranes of this type allows these systems to be adapted for diverse folded protein sensing applications, for example.

Non-naturally occurring amphiphiles and amphiphiles which form an amphiphilic membrane layer are known in the art and include, for example, block copolymers (Gonzalez-Perez et al., Langmuir, 2009, 25, 10447-10450). The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane may be chosen one of the membranes disclosed in PCT/GB2013/052767, hereby incorporated by reference in its entirety. The amphiphilic molecules may be chemically-modified or functionalised to facilitate coupling an insertion of the nanostructure. The membrane can comprise both a lipid and an amphiphilic polymer such as disclosed in PCT/US2016/040665.

Polymer-based membrane may be formed of any suitable material. Typically, synthetic membranes are composed of amphiphilic synthetic block copolymers. Examples of hydrophilic block copolymers are poly(ethylene glycol) (PEG/PEO) or poly(2-methyloxazoline), while examples of hydrophobic blocks are polydimethylsiloxane (PDMS), poly(caprolactone (PCL), poly(lactide) (PLA), or poly(methyl methacrylate) (PMMA). In embodiments, the polymer membrane used may be formed from the amphiphilic block copolymer poly 2-(methacryloyloxy)ethyl phosphorylcholine-b-disisopropylamino)ethyl methacrylate (PMPC-b-PDPA). DNA nanopores may be inserted into the walls of such polymersomes through incubation. Without wishing to be bound by theory, it is believed that one process of insertion broadly involves first steps of membrane tethering, followed by second steps of orientation of the DNA pore relative to the membrane to achieve complete insertion. This however requires lipid membrane anchors to be comprised within, or at least attached to, the pores, without which insertion does not take place. For alternative configurations of the invention, particularly where nucleic acid analogues (e.g. XNAs) comprise some or all of the backbone of the scaffold and/or staple strands, it is envisaged that more complex insertional dynamics may be observed. It will be appreciated that where the scaffold and staple strands incorporate synthetic biopolymer backbone constituents capable of mediating a hydrophobic interaction with a membrane, the insertion process may or may not involve initial stages of coplanar alignment with the membrane followed by a phase of insertion.

The membrane is typically planar, although in certain embodiments it may be curved or shaped. Amphiphilic membrane layers may also be supported. Suitably the membrane is a lipid bilayer or monolayer. Methods for forming lipid bilayers are known in the art such as disclosed in International Application Number PCT/GB2008/000563. Lipid bilayers are commonly formed by the method of Montal and Mueller (Proc. Natl. Acad. Sci. USA., 1972; 69:3561-3566).

In another embodiment of the invention, the membrane may comprise a solid state layer. Solid state layers can be formed from organic and/or inorganic materials including, but not limited to, microelectronic materials, insulating materials such as Si3N4, Al₂O₃, and SiO, glasses, organic and inorganic polymers such as polyamide and plastics. The solid state layer may be formed from graphene such as disclosed in PCT/US2008/010637. The membrane may be provided with one or more apertures or through-holes of nanometre scale dimensions extending from one side of the membrane to the other. The internal walls of the solid state aperture may be coated with a lipid such as disclosed in US2017/0023544 or chemically functionalized such as disclosed in PCT/US2008/063066, so as to facilitate suitable anchoring of the nanostructures of the invention to the solid state layer.

The ability to adapt the nanopores of the present invention such that they may be inserted into membranes of varying thickness is a significant advantage. In embodiments of the invention one or more scaffold strands may be utilised to form a coil or stacked ring structure in order to vary the depth of the pore and, thus, its ability to be adapted to span membranes of a given thickness (as shown in FIG. 1). In any case, a significant advantage of the nanopores of the present invention is that length of the channel is not required to be considerably greater than the width of the membrane. This ensures that non-specific binding of analyte to the interior walls of thechannel is reduced. In addition, the time taken for ions to traverse the membrane, via the pore, is reduced leading to faster readouts.

A further advantage of the nanostructures of the present invention is that they are more structurally stable compared to nucleic acid nanopores that are comprised of a plurality of bundled duplexes that are oriented perpendicularly to the plane of the membrane. As mentioned previously, structural instability due to planar membrane pressures can lead to ion leakage across the pore, reducing accuracy and fidelity of signal, and significantly increasing background noise in sensors that comprise such nanopores. The nanostructures of the present invention are more resistant to the compressive membrane pressures leading to improved structural integrity, greater consistency of pore diameter (i.e. homogeneity of pores and signal) and reduced ion leakage.

In a specific embodiment of the present invention the nanostructures of the invention comprise a membrane spanning nanopore which is functionalised by addition of one or more target molecule binding moieties. The one or more target binding moieties may be placed on one or more modules of the nanopore. There may be one or more binding moieties placed on a single module of the nanopore, or alternatively, there may be one or more binding moieties placed on a different module of the nanopore. The simplicity of functionalising individual modules of the nanopore provides a particular advantage that a specific arrangement or number of binding moieties may be provided on the nanopore allowing more control and efficiency of binding of a target module than offered by prior art nanopores. It is envisioned, for example, that specific binding moieties may be positioned on opposite sides of the opening of the channel to ensure that when a target is captured it more effectively blocks flow through the channel.

The target may comprise an analyte in solution or a molecule that is capable of generating a detectable signal, such as a fluorophore or radiolabel. Binding of the analyte will serve to fully or partially occlude the channel of the nanopore such that a detectable electrical readout can be obtained. The target molecule binding moiety may be comprised within or attached to one or more polynucleotide strands that contribute to the formation of the nanopore, or alternatively added post hoc by binding to an exposed thiol group or biotin tag (via avidin or an analogue thereof) incorporated within the one or more polynucleotide strands. The binding moiety may comprise a polynucleotide or a polypeptide that is capable of binding to an analyte present in a solution that surrounds the membrane-embedded nanopore. Where the binding molecule comprises a polypeptide that is tethered to the nanopore, either within the channel or proximate to the cis or trans side of the nanopore, it may be selected from one of the group consisting of:
I. an enzyme—including a polymerase, a helicase, a gyrase, and a telomerase, as well as nucleic acid binding sub domains or derivatives thereof
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including drugs, fluorophores, metabolites and chemokines; and
VI. signalling molecules and/or polypeptide receptors thereof, including binding domains of receptors, and receptor complexes.

The nanopore structures of the present invention are suited to use in sensor applications that allow for the detection of a diverse range of potential analytes that may exist in a solution that is under test. Exemplary analytes may include:
peptides/polypeptides/proteins-folded, partially/completely unfolded;
enzymes;
protein/nucleic acid constructs;
molecules defined by size;
macromolecules within specified size ranges, for example, in ranges selected from: 1-10 KD, 1-50 kD, 1-100 kD, 10-50 kD, 10-100 kD, 20-50 kD, and 20-100 kD;
multi-protein complexes;
antigens;
glycoproteins;
carbohydrates;
biopolymers;
toxins;
metabolites and by products thereof;
cytokines;
nucleic acids.

The nanopore structures of the present invention may be incorporated within a plurality of improved devices and sensors. Such devices and sensors are useful in applications requiring to sensing and characterization of a variety of materials and analytes. By way of non-limiting example, particularly useful applications, including genome sequencing, protein sequencing, other biomolecular sequencing, and detection of ions, molecules, chemicals, small molecules, biomolecules, metal atoms, contaminants, polymers, nanoparticles etc. Such detecting and characterizing can, in turn, be used to diagnose diseases, in drug development, to identify contamination or adulteration or food or water supplies, and in quality control and standardization.

According to exemplary embodiments of the present invention as described, a sensor device typically comprises a substrate that includes a membrane into which one or more nanopores are embedded. The substrate is placed to facilitate contact with a fluid (optionally an electrolytic solution) which comprises an analyte. At least one, and optionally a plurality of, device(s) are positioned relative to the substrate, wherein a given device generates a signal (e.g. mechanical, electrical, and/or optical) in response to detecting binding to and/or passage through the nanopore(s) of one or one or more analytes. The plurality of devices can be greater than 2 and as many as 100, or as many as 20, or as many as 10, or between 2 and 8 devices. Each device may be selected from one or more of the group consisting of: a field effect sensor; a plasmonic sensor; a laser based sensor; an interferometric sensor; a wave-guide sensor; a cantilever sensor; an acoustic sensor; a quartz crystal microbalance (QCM) sensor; an ultrasonic sensor; a mechanical sensor; a thermal sensor; an optical dye based sensor; a fluorimetric sensor; a calorimetric sensor; a luminometric sensor; a graphene sensor; a quantum dot sensor; a quantum-well sensor; a photoelectric sensor; a 2D material sensor; a nanotube or nanowire sensor; an enzymatic sensor; an electrochemical sensor, including a FET or BioFET sensor; a potentiometric sensor; a conductometric sensor; a capacitive sensors; and an electron-spin sensor. The devices may cooperate in the form of arrays allowing for multiplexed testing of multiple analytes. The sensor devices may further comprise special purpose hardware and systems (e.g., circuitry, processors, memory, GUIs etc.) that perform the specified functions or acts, or combinations of special purpose hardware and computer instructions, in order to render a functioning sensor device capable of providing a meaningful readout to a user.

Hence, according to embodiments of the invention suitably configured nanopore devices may enable a variety of different types of sensor measurements to be made. This includes without limitation: electrical measurements and optical measurements. A suitable optical method involving the measurement of fluorescence is disclosed by J. Am. Chem. Soc. 2009, 131 1652-1653. Possible electrical measurements include: current measurements, impedance measurements, tunnelling measurements (Ivanov A P et al., Nano Lett. 2011 Jan. 12; 11 (1): 279-85), and FET measurements (International Application WO 2005/124888). Optical measurements may be combined with electrical measurements (Soni G V et al., Rev Sci Instrum. 2010 January; 81 (1): 014301). The measurement may be a transmembrane current measurement such as measurement of ionic current flowing through the pore.

The invention is further illustrated by the following non-limiting examples.

### EXAMPLES

### Example 1

### Construction of Nucleic Acid Planar Ring Nanopore

This nanostructure design was aimed at creating a novel large nanopore (FIG. 1a) that can embed in a semi-fluid lipid membrane and specifically permit the selective passage of analyte (FIG. 1b) and ions through the membrane barrier. To enable the insertion of the negatively charged DNA nanopore to the lipid bilayer, hydrophobic cholesterol tags were also incorporated at selected positions in the structure—in this case at six positions equally spaced around the periphery of the nanopore (FIG. 1c).

The main structure body was composed of DNA molecules which were designed with the aid of the caDNAno software (see above) and then rationally modified to fulfil both the structural and functional needs.

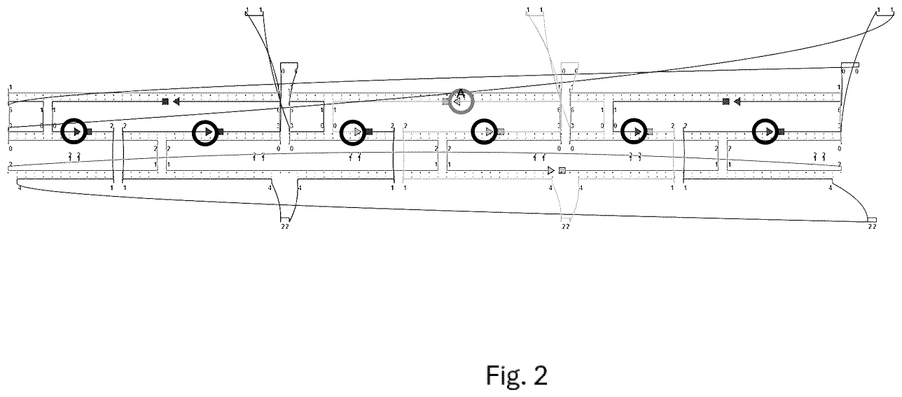

As shown in FIG. 2, the structure was composed of one long scaffold and nine short staple strands. The long scaffold serves as a template for the assembly of the structure, and its laypath defines the overall shape and size of the nanopore. The short staples were modified with cholesterol tags, fluorophores and biotin moieties, with the functionalization achieved through the folding process.

As shown in FIGS. 1 and 2, the minimal design principle of the pore structure avoided extra parts, modifications or extraneous additions to the overall structure. Notably, the design of the pore was minimized to only have the 10 nm wide transmembrane portion and its 7 nm height defined by the three DNA duplexes was only slightly greater than the thickness of the lipid bilayer (5 nm). This novel design possesses many advantages over all the existing conventional pores based on large DNA capped origami structures. In particular, the current design enhances stability of the nanostructure by reducing electrostatic repulsion within the structure leading to considerably reduced folding times. In certain instances, folding times of a few hours are observed rather than the conventional expectation of several days.

TABLE 1
Scaffold strands for nucleic acid planar
ring nanopore:

| | |
|---|---|
| S1 (SEQ | TGATAGACGGTTTTTCGCCCTTTGACGTTGGAGTCCA |
| ID NO: 1) | CGTTCTTTAATAGTGGACTCTTGTTCCAAACTGGAAC |
| | AACACTCAACCCTATCTCGGGCTATTCTTTTGATTTA |
| | TAAGGGATTTTGCCGATTTCGGAACCACCATCAAACA |
| | GGATTTTCGCCTG |
| S2 (SEQ | CTGGGGCAAACCAGCGTGGACCGCTTGCTGCAACTCT |
| ID NO: 2) | CTCAGGGCCAGGCGGTGAAGGGCAATCAGCTGTTGCC |
| | CGTCTCACTGGTGAAAAGAAAAACCACCCTGGCGCCC |
| | AATACGCAAACCGCCT |
| L (SEQ | GGTTTGCCCCAGCAGGCGAAAATCC |
| ID NO: 3) | |

Strand S2 was 5′-phosphorylated.

TABLE 2
Staple strands for nucleic acid planar ring
nanopore:

| | |
|---|---|
| R1 (SEQ | CACCAGTGAGACGGATTG |
| ID NO: 4) | |
| R2 (SEQ | AGTCCACTATTAAATCCA |
| ID NO: 5) | |
| R3 (SEQ | ATCCTGTTTGATGGATCG |
| ID NO: 6) | |
| R4 (SEQ | GCAAAAATCAAAAGAATTGCCCGAGATAGGGGGCGAC |
| ID NO: 7) | AAAGTTGAGT |
| R5 (SEQ | ACGTAAAACCGTCTATTTAGGCGGTTTGCGTGCCTGT |
| ID NO: 8) | CACCATTGGG |
| R6 (SEQ | CCCTGCCCTGAGAGAGTTGCAGCAAGCGGTCCTTATA |
| ID NO: 9) | ATCCCACGCT |
| R7 (SEQ | CGCCAGGGTTTTGGACGAACGTTTTGGTTTTTCTTTT |
| ID NO: 10) | |
| R8 (SEQ | GTTGTTCCTTTCCGAATGGTTTTTAGTTTGGAACAAG |
| ID NO: 11) | |
| R9 (SEQ | GGTTTGCCTTTAGCTGGCAACTTTCCAGCAGGCGAAA |
| ID NO: 12) | |

Strands R1~R6 were modified with 3′-cholesterol tags, and strand R7 was modified with 5′-Atto647N.

### Example 2

### Assembly and Membrane Insertion of Nucleic Acid Planar Ring Nanopore

The DNA ring structure was folded from ten DNA strands, i.e. one long scaffold strand S and nine short staple strands.

Firstly, the long scaffold strand S was prepared from the enzymatic ligation of two shorter strands S1 and S2 (SEQ ID NO: 1&2). In a typical reaction, 200 pmol of S1 and S2 (5′-phosphorylated) were mixed with 240 pmol of linking strand L (SEQ ID NO: 3), and annealed in a 1×T4 DNA ligase buffer from 65° C. to 25° C. in 2 hours. The T4 DNA ligase (NEB) was added, and incubated overnight at 16° C. and then heated at 65° C. for 20 min. The ligated product was purified by 2% agarose gel. The harvested strand S was then quantified by the UV absorption at 260 nm and was stored at −20° C. for the further use.

Next, to prepare DNA ring structures, with or without cholesterol-modified (Chl-DNA) and dye-labeled (Atto647N) strands, 10 pmol of scaffold strand S and 5× excess corresponding staples were mixed in the folding buffer (0.5×TAE with 12 mM MgCl2) and annealed with a 2-hour folding program (heating at 85° C. for 5 min, temperature ramping from 65° C. to 25° C. at 1° C./2.5 min, from 25° C. to 5° C. at 1° C./0.5 min) on a thermocycler. The folded structures were then characterized and purified with 1.5% agarose gel.

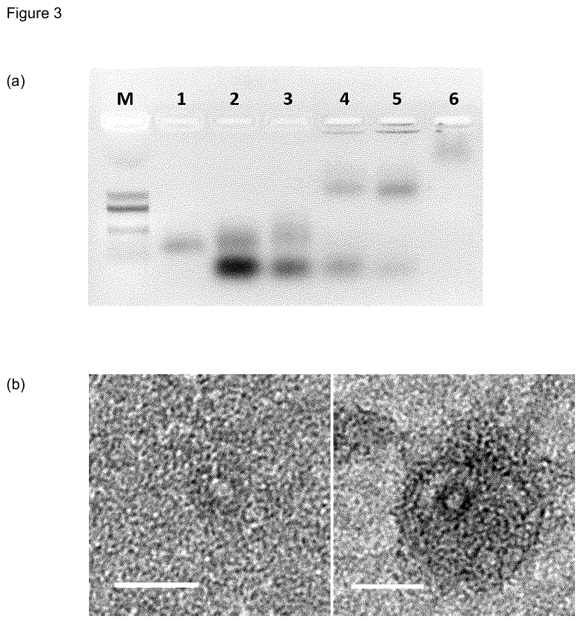

M-100 bp DNA ladder was used as the reference standard, lane 1-scaffold only, lane 2-linear structure (L, linearized ring), lane 3-ring structure (rOc, without cholesterol); lane 4 and 5-ring structure bearing four and six cholesterol tags (r4c and r6c), respectively; lane 6-cholesterol-DNA only. Gels were run at 65 V for 1 h at 8° C. DNA bands were visualized by staining with ethidium bromide solution and ultraviolet illumination (see FIG. 3a)

The nanostructures were further analysed by transmission electron microscopy (TEM) in order to verify their stability before and after insertion into a small unilamellar vesicle (SUV) membrane (see FIG. 3b).

To check the binding ability of the cholesterol-bearing DNA ring structures with the lipid membrane, two characterization methods, TEM and confocal microscope, were used.

For the TEM samples (see FIG. 3b), the DNA ring structures, carrying cholesterol-labeled staples, were incubated with 1-palmitoyl-2-oleoyl-sn-glycero-3-phosphocholine (POPC) SUVs 1× incubation buffer (0.5×TAE with 500 mM NaCl) for 20 min and then were used to prepare TEM grids and checked by TEM observation. The POPC SUVs solution was prepared by adding 50 μL of 20 mg/mL POPC (in chloroform) in a 2 mL glass vial and dried by argon airflow, and resuspended with 1× incubation buffer and sonicated for 30 min.

For the confocal samples (see FIG. 4), the DNA ring structures, carrying cholesterol-labeled staples and an Atto647N-labeled staple, were incubated with POPC GUVs in 1× incubation buffer for 30 min, and the incubated samples were then observed with an inverted confocal microscope (Leica). The POPC GUVs were prepared with the electroformation method. Briefly, two droplets of POPC solution (10 mg/mL in chloroform, 3 μL each) were added onto a ITO glass slide. After 5 min, the solvent was evaporated and two dried lipid film patches were formed. The glass slide was then applied on a Vesicle Prep device (Nanion). After adding 600 μL sucrose solution (1 M in water) to the lipid film patches confined by two O-rings, another ITO glass slide was applied from the top, and a sealed chamber was thus formed. An alternating electric field was applied between the two slides by running a programmed protocol, briefly, 3 V, at 5 Hz for 120 min. After the program was done, the solution was collected and then stored at 4° C. for the future use.

### Example 3

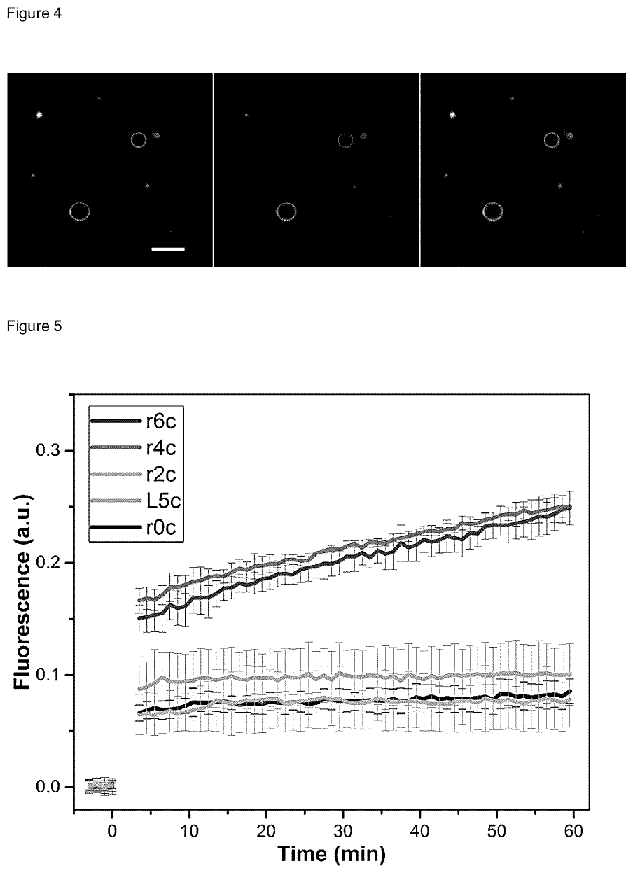

The DNA Ring Mediated Dye Releasing Tests with POPC SUVs (FIG. 5)

Firstly, the POPC SUVs was prepared with a modified method from the last section, not using the conventional 1× incubation buffer, but changing to 1× dye-containing buffer (0.5 TAE with 500 mM 6-Carboxyfluorescein (6-CF)), to resuspend the lipid film to form the dye-loaded SUVs. The excess dyes were then removed by G-25 gel filtration. The purified SUVs solution was diluted to 10 times volume in 1× incubation buffer for further use.

Next, the dye-loaded SUVs solution was used to incubate with different DNA ring structures, varying from the shapes of the structure or the number of cholesterol tags bearing on the structures. The dye-releasing tests were done on a fluorometer (Varian) by measuring the fluorescence intensity (λex=492 nm, λem=517 nm).

### Example 4

Functional Nanopores Mediate the Influx of Dye into GUVs Vesicles in Size-Dependent Fashion

The DNA ring structures, carrying cholesterol-labeled staples and an Atto647N-labeled staple, were added to POPC GUVs in 1× incubation buffer and mixed gently in a dish with a wait for 2 minutes for the GUVs to settle down for the confocal observation.

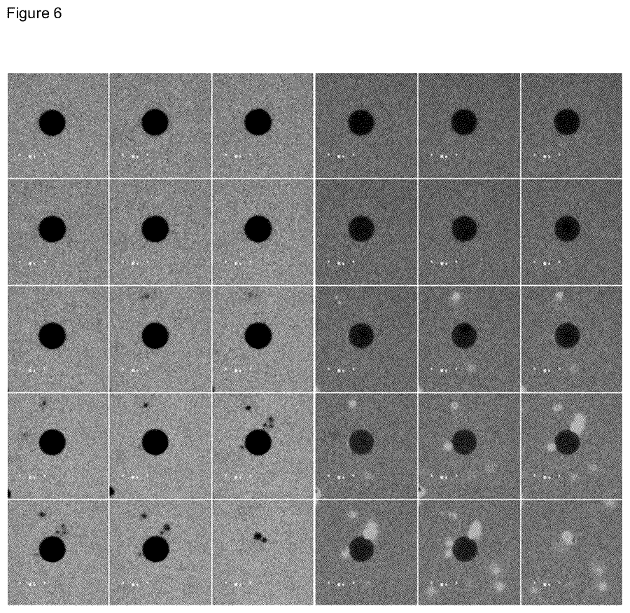

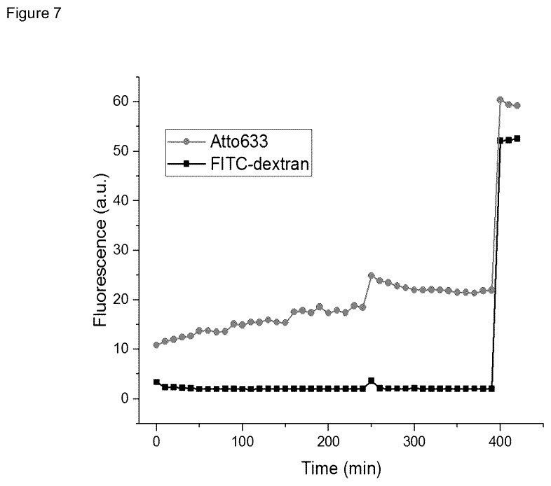

To check whether the DNA ring structures can perforate the lipid membrane as transmembrane channels to transport molecular cargo, we used two kinds of dyes, FITC-dextran (green, MW=500 kD) and Atto633 (red), and checked with fluorescence intensity whether the dyes flow into the lumen of the GUVs. The dye Atto633 is a small molecule and expected to pass the ASUpore, while FITC-dextran (MW=500 kDa) with a nominal diameter of ˜ 29.4 nm is not able to pass through the channel of DNA nanopore (nominal diameter ˜10 nm). The fluorescence intensity of the two dyes was measured by examining the same microscopic region-of-interest within the GUV lumen. As shown in FIGS. 6 and 7, the intensity of the Atto633 increased with time, while the intensity of the FITC-dextran remained very low during the whole measurement over 5 hours.

### Example 5

### Construction of Nucleic Acid Polygonal Ring Nanopore

The design of generic polygonal pores is inspired by oligomeric protein pores and relies on a polygonal framework of multiple modular blocks. Referring to FIG. 8, the blocks or modules of the polygonal pores are suitably composed of DNA helix bundles that are arranged such a major portion of the scaffold polynucleotide chains run substantially parallel to the membrane once inserted. The polygonal nanopores of the present invention may have any suitable shape, including triangles, and quadrilaterals such as squares or rectangles, pentagons, hexagons, or trapezoid shape. The modules may have tuneable side length such as 10 or 20 nm as shown in FIG. 8. The modular blocks are composed of bundles of parallel aligned DNA duplexes. In FIG. 8A to D (triangle, square pore, pentagon and hexagon respectively), the units are made up of bundles of 3×2 duplexes having a side length of approximately 10 nm. By comparison, FIG. 8E to 8F (triangle and square respectively), the units are composed of 3×4 duplexes having a side length of approximately 20 nm. In this example, the frames enclose a channel area of about 43 to about 400 nm².

The modular blocks are connected via single stranded DNA linkers at innermost duplex layer of the blocks. To avoid instability and/or collapse of the pore and ensure the overall structure, rigid duplex stabilizers (sub-modules) are positioned between the blocks at the second duplex layer of square, pentagon and hexagon pores.

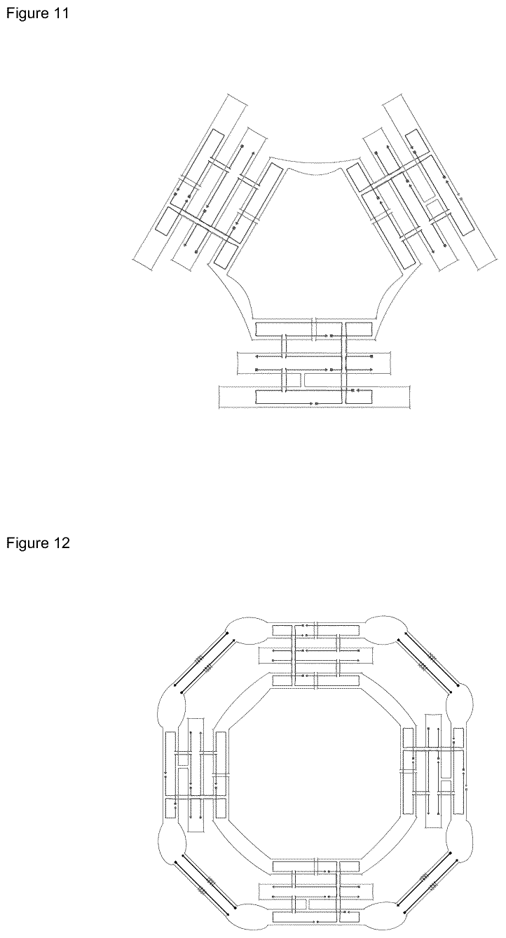

The two-dimensional DNA map of the 10 nm triangle and the 10 nm square pore frame are shown in FIGS. 11 and 12.

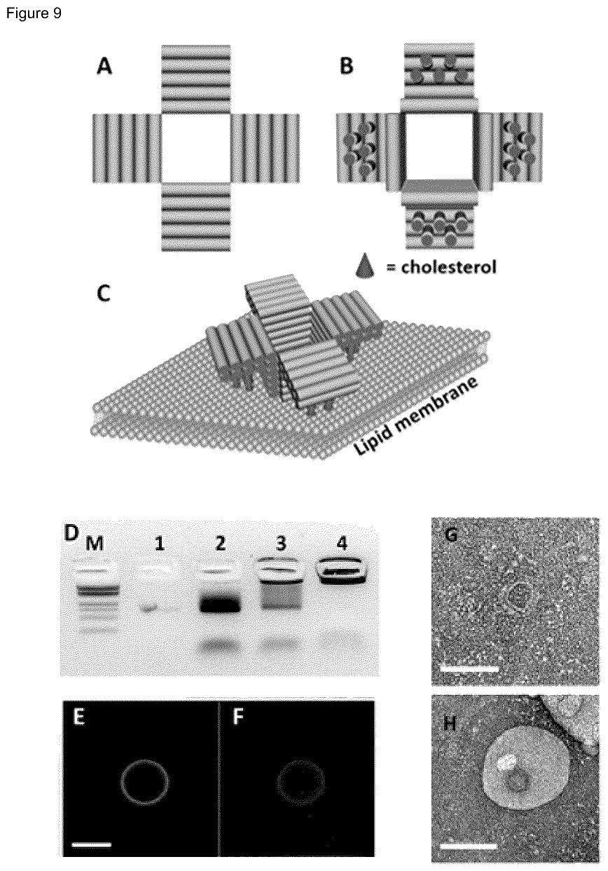

As best seen in FIGS. 9B and 9C, as an additional component, the pores form a channel that punctures the membrane on insertion, as well as lipid membrane anchors.

TABLE 3
Scaffold strands for nucleic acid polygonal nanopore:

| | |
|---|---|
| M13 | AATGCTACTACTATTAGTAGAATTGATGCCACCTTTTCAGCTCGCGCCCCAAATGAAAA |
| (SEQ ID NO: 13) | TATAGCTAAACAGGTTATTGACCATTTGCGAAATGTATCTAATGGTCAAACTAAATCTAC |
| | TCGTTCGCAGAATTGGGAATCAACTGTTATATGGAATGAAACTTCCAGACACCGTACTT |
| | TAGTTGCATATTTAAAACATGTTGAGCTACAGCATTATATTCAGCAATTAAGCTCTAAGC |
| | CATCCGCAAAAATGACCTCTTATCAAAAGGAGCAATTAAAGGTACTCTCTAATCCTGAC |
| | CTGTTGGAGTTTGCTTCCGGTCTGGTTCGCTTTGAAGCTCGAATTAAAACGCGATATTT |
| | GAAGTCTTTCGGGCTTCCTCTTAATCTTTTTGATGCAATCCGCTTTGCTTCTGACTATAA |
| | TAGTCAGGGTAAAGACCTGATTTTTGATTTATGGTCATTCTCGTTTTCTGAACTGTTTAA |
| | AGCATTTGAGGGGGATTCAATGAATATTTATGACGATTCCGCAGTATTGGACGCTATCC |
| | AGTCTAAACATTTTACTATTACCCCCTCTGGCAAAACTTCTTTTGCAAAAGCCTCTCGCT |
| | ATTTTGGTTTTTATCGTCGTCTGGTAAACGAGGGTTATGATAGTGTTGCTCTTACTATGC |
| | CTCGTAATTCCTTTTGGCGTTATGTATCTGCATTAGTTGAATGTGGTATTCCTAAATCTC |
| | AACTGATGAATCTTTCTACCTGTAATAATGTTGTTCCGTTAGTTCGTTTTATTAACGTAGA |
| | TTTTTCTTCCCAACGTCCTGACTGGTATAATGAGCCAGTTCTTAAAATCGCATAAGGTAA |
| | TTCACAATGATTAAAGTTGAAATTAAACCATCTCAAGCCCAATTTACTACTCGTTCTGGT |
| | GTTTCTCGTCAGGGCAAGCCTTATTCACTGAATGAGCAGCTTTGTTACGTTGATTTGGG |
| | TAATGAATATCCGGTTCTTGTCAAGATTACTCTTGATGAAGGTCAGCCAGCCTATGCGC |
| | CTGGTCTGTACACCGTTCATCTGTCCTCTTTCAAAGTTGGTCAGTTCGGTTCCCTTATG |
| | ATTGACCGTCTGCGCCTCGTTCCGGCTAAGTAACATGGAGCAGGTCGCGGATTTCGAC |
| | ACAATTTATCAGGCGATGATACAAATCTCCGTTGTACTTTGTTTCGCGCTTGGTATAATC |
| | GCTGGGGGTCAAAGATGAGTGTTTTAGTGTATTCTTTTGCCTCTTTCGTTTTAGGTTGG |
| | TGCCTTCGTAGTGGCATTACGTATTTTACCCGTTTAATGGAAACTTCCTCATGAAAAAGT |
| | CTTTAGTCCTCAAAGCCTCTGTAGCCGTTGCTACCCTCGTTCCGATGCTGTCTTTCGCT |
| | GCTGAGGGTGACGATCCCGCAAAAGCGGCCTTTAACTCCCTGCAAGCCTCAGCGACC |
| | GAATATATCGGTTATGCGTGGGCGATGGTTGTTGTCATTGTCGGCGCAACTATCGGTAT |
| | CAAGCTGTTTAAGAAATTCACCTCGAAAGCAAGCTGATAAACCGATACAATTAAAGGCT |
| | CCTTTTGGAGCCTTTTTTTTGGAGATTTTCAACGTGAAAAAATTATTATTCGCAATTCCTT |
| | TAGTTGTTCCTTTCTATTCTCACTCCGCTGAAACTGTTGAAAGTTGTTTAGCAAAATCCC |
| | ATACAGAAAATTCATTTACTAACGTCTGGAAAGACGACAAAACTTTAGATCGTTACGCTA |
| | ACTATGAGGGCTGTCTGTGGAATGCTACAGGCGTTGTAGTTTGTACTGGTGACGAAAC |
| | TCAGTGTTACGGTACATGGGTTCCTATTGGGCTTGCTATCCCTGAAAATGAGGGTGGT |
| | GGCTCTGAGGGTGGCGGTTCTGAGGGTGGCGGTTCTGAGGGTGGCGGTACTAAACCT |
| | CCTGAGTACGGTGATACACCTATTCCGGGCTATACTTATATCAACCCTCTCGACGGCAC |
| | TTATCCGCCTGGTACTGAGCAAAACCCCGCTAATCCTAATCCTTCTCTTGAGGAGTCTC |
| | AGCCTCTTAATACTTTCATGTTTCAGAATAATAGGTTCCGAAATAGGCAGGGGGCATTA |
| | ACTGTTTATACGGGCACTGTTACTCAAGGCACTGACCCCGTTAAAACTTATTACCAGTA |
| | CACTCCTGTATCATCAAAAGCCATGTATGACGCTTACTGGAACGGTAAATTCAGAGACT |
| | GCGCTTTCCATTCTGGCTTTAATGAGGATTTATTTGTTTGTGAATATCAAGGCCAATCGT |
| | CTGACCTGCCTCAACCTCCTGTCAATGCTGGCGGCGGCTCTGGTGGTGGTTCTGGTG |
| | GCGGCTCTGAGGGTGGTGGCTCTGAGGGTGGCGGTTCTGAGGGTGGCGGCTCTGAG |
| | GGAGGCGGTTCCGGTGGTGGCTCTGGTTCCGGTGATTTTGATTATGAAAAGATGGCAA |
| | ACGCTAATAAGGGGGCTATGACCGAAAATGCCGATGAAAACGCGCTACAGTCTGACGC |
| | TAAAGGCAAACTTGATTCTGTCGCTACTGATTACGGTGCTGCTATCGATGGTTTCATTG |
| | GTGACGTTTCCGGCCTTGCTAATGGTAATGGTGCTACTGGTGATTTTGCTGGCTCTAAT |
| | TCCCAAATGGCTCAAGTCGGTGACGGTGATAATTCACCTTTAATGAATAATTTCCGTCA |
| | ATATTTACCTTCCCTCCCTCAATCGGTTGAATGTCGCCCTTTTGTCTTTGGCGCTGGTA |
| | AACCATATGAATTTTCTATTGATTGTGACAAAATAAACTTATTCCGTGGTGTCTTTGCGT |
| | TTCTTTTATATGTTGCCACCTTTATGTATGTATTTTCTACGTTTGCTAACATACTGCGTAA |
| | TAAGGAGTCTTAATCATGCCAGTTCTTTTGGGTATTCCGTTATTATTGCGTTTCCTCGGT |
| | TTCCTTCTGGTAACTTTGTTCGGCTATCTGCTTACTTTTCTTAAAAAGGGCTTCGGTAAG |
| | ATAGCTATTGCTATTTCATTGTTTCTTGCTCTTATTATTGGGCTTAACTCAATTCTTGTGG |
| | GTTATCTCTCTGATATTAGCGCTCAATTACCCTCTGACTTTGTTCAGGGTGTTCAGTTAA |
| | TTCTCCCGTCTAATGCGCTTCCCTGTTTTTATGTTATTCTCTCTGTAAAGGCTGCTATTT |
| | TCATTTTTGACGTTAAACAAAAAATCGTTTCTTATTTGGATTGGGATAAATAATATGGCT |
| | GTTTATTTTGTAACTGGCAAATTAGGCTCTGGAAAGACGCTCGTTAGCGTTGGTAAGAT |
| | TCAGGATAAAATTGTAGCTGGGTGCAAAATAGCAACTAATCTTGATTTAAGGCTTCAAAA |
| | CCTCCCGCAAGTCGGGAGGTTCGCTAAAACGCCTCGCGTTCTTAGAATACCGGATAAG |
| | CCTTCTATATCTGATTTGCTTGCTATTGGGCGCGGTAATGATTCCTACGATGAAAATAAA |
| | AACGGCTTGCTTGTTCTCGATGAGTGCGGTACTTGGTTTAATACCCGTTCTTGGAATGA |
| | TAAGGAAAGACAGCCGATTATTGATTGGTTTCTACATGCTCGTAAATTAGGATGGGATA |
| | TTATTTTTCTTGTTCAGGACTTATCTATTGTTGATAAACAGGCGCGTTCTGCATTAGCTG |
| | AACATGTTGTTTATTGTCGTCGTCTGGACAGAATTACTTTACCTTTTGTCGGTACTTTAT |
| | ATTCTCTTATTACTGGCTCGAAAATGCCTCTGCCTAAATTACATGTTGGCGTTGTTAAAT |
| | ATGGCGATTCTCAATTAAGCCCTACTGTTGAGCGTTGGCTTTATACTGGTAAGAATTTG |
| | TATAACGCATATGATACTAAACAGGCTTTTTCTAGTAATTATGATTCCGGTGTTTATTCTT |
| | ATTTAACGCCTTATTTATCACACGGTCGGTATTTCAAACCATTAAATTTAGGTCAGAAGA |
| | TGAAATTAACTAAAATATATTTGAAAAAGTTTTCTCGCGTTCTTTGTCTTGCGATTGGATT |
| | TGCATCAGCATTTACATATAGTTATATAACCCAACCTAAGCCGGAGGTTAAAAAGGTAG |
| | TCTCTCAGACCTATGATTTTGATAAATTCACTATTGACTCTTCTCAGCGTCTTAATCTAA |
| | GCTATCGCTATGTTTTCAAGGATTCTAAGGGAAAATTAATTAATAGCGACGATTTACAGA |
| | AGCAAGGTTATTCACTCACATATATTGATTTATGTACTGTTTCCATTAAAAAAGGTAATTC |
| | AAATGAAATTGTTAAATGTAATTAATTTTGTTTTCTTGATGTTTGTTTCATCATCTTCTTTT |
| | GCTCAGGTAATTGAAATGAATAATTCGCCTCTGCGCGATTTTGTAACTTGGTATTCAAA |
| | GCAATCAGGCGAATCCGTTATTGTTTCTCCCGATGTAAAAGGTACTGTTACTGTATATTC |
| | ATCTGACGTTAAACCTGAAAATCTACGCAATTTCTTTATTTCTGTTTTACGTGCAAATAAT |
| | TTTGATATGGTAGGTTCTAACCCTTCCATTATTCAGAAGTATAATCCAAACAATCAGGAT |
| | TATATTGATGAATTGCCATCATCTGATAATCAGGAATATGATGATAATTCCGCTCCTTCT |
| | GGTGGTTTCTTTGTTCCGCAAAATGATAATGTTACTCAAACTTTTAAAATTAATAACGTTC |
| | GGGCAAAGGATTTAATACGAGTTGTCGAATTGTTTGTAAAGTCTAATACTTCTAAATCCT |
| | CAAATGTATTATCTATTGACGGCTCTAATCTATTAGTTGTTAGTGCTCCTAAAGATATTTT |
| | AGATAACCTTCCTCAATTCCTTTCAACTGTTGATTTGCCAACTGACCAGATATTGATTGA |
| | GGGTTTGATATTTGAGGTTCAGCAAGGTGATGCTTTAGATTTTTCATTTGCTGCTGGCT |
| | CTCAGCGTGGCACTGTTGCAGGCGGTGTTAATACTGACCGCCTCACCTCTGTTTTATCT |
| | TCTGCTGGTGGTTCGTTCGGTATTTTTAATGGCGATGTTTTAGGGCTATCAGTTCGCGC |
| | ATTAAAGACTAATAGCCATTCAAAAATATTGTCTGTGCCACGTATTCTTACGCTTTCAGG |
| | TCAGAAGGGTTCTATCTCTGTTGGCCAGAATGTCCCTTTTATTACTGGTCGTGTGACTG |
| | GTGAATCTGCCAATGTAAATAATCCATTTCAGACGATTGAGCGTCAAAATGTAGGTATTT |
| | CCATGAGCGTTTTTCCTGTTGCAATGGCTGGCGGTAATATTGTTCTGGATATTACCAGC |
| | AAGGCCGATAGTTTGAGTTCTTCTACTCAGGCAAGTGATGTTATTACTAATCAAAGAAG |
| | TATTGCTACAACGGTTAATTTGCGTGATGGACAGACTCTTTTACTCGGTGGCCTCACTG |
| | ATTATAAAAACACTTCTCAGGATTCTGGCGTACCGTTCCTGTCTAAAATCCCTTTAATCG |
| | GCCTCCTGTTTAGCTCCCGCTCTGATTCTAACGAGGAAAGCACGTTATACGTGCTCGTC |
| | AAAGCAACCATAGTACGCGCCCTGTAGCGGCGCATTAAGCGCGGCGGGTGTGGTGGT |
| | TACGCGCAGCGTGACCGCTACACTTGCCAGCGCCCTAGCGCCCGCTCCTTTCGCTTTC |
| | TTCCCTTCCTTTCTCGCCACGTTCGCCGGCTTTCCCCGTCAAGCTCTAAATCGGGGGC |
| | TCCCTTTAGGGTTCCGATTTAGTGCTTTACGGCACCTCGACCCCAAAAAACTTGATTTG |
| | GGTGATGGTTCACGTAGTGGGCCATCGCCCTGATAGACGGTTTTTCGCCCTTTGACGT |
| | TGGAGTCCACGTTCTTTAATAGTGGACTCTTGTTCCAAACTGGAACAACACTCAACCCT |
| | ATCTCGGGCTATTCTTTTGATTTATAAGGGATTTTGCCGATTTCGGAACCACCATCAAAC |
| | AGGATTTTCGCCTGCTGGGGCAAACCAGCGTGGACCGCTTGCTGCAACTCTCTCAGG |
| | GCCAGGCGGTGAAGGGCAATCAGCTGTTGCCCGTCTCACTGGTGAAAAGAAAAACCA |
| | CCCTGGCGCCCAATACGCAAACCGCCTCTCCCCGCGCGTTGGCCGATTCATTAATGCA |
| | GCTGGCACGACAGGTTTCCCGACTGGAAAGCGGGCAGTGAGCGCAACGCAATTAATG |
| | TGAGTTAGCTCACTCATTAGGCACCCCAGGCTTTACACTTTATGCTTCCGGCTCGTATG |
| | TTGTGTGGAATTGTGAGCGGATAACAATTTCACACAGGAAACAGCTATGACCATGATTA |
| | CGAATTCGAGCTCGGTACCCGGGGATCCTCTAGAGTCGACCTGCAGGCATGCAAGCTT |
| | GGCACTGGCCGTCGTTTTACAACGTCGTGACTGGGAAAACCCTGGCGTTACCCAACTT |
| | AATCGCCTTGCAGCACATCCCCCTTTCGCCAGCTGGCGTAATAGCGAAGAGGCCCGCA |
| | CCGATCGCCCTTCCCAACAGTTGCGCAGCCTGAATGGCGAATGGCGCTTTGCCTGGTT |
| | TCCGGCACCAGAAGCGGTGCCGGAAAGCTGGCTGGAGTGCGATCTTCCTGAGGCCGA |
| | TACTGTCGTCGTCCCCTCAAACTGGCAGATGCACGGTTACGATGCGCCCATCTACACC |
| | AACGTGACCTATCCCATTACGGTCAATCCGCCGTTTGTTCCCACGGAGAATCCGACGG |
| | GTTGTTACTCGCTCACATTTAATGTTGATGAAAGCTGGCTACAGGAAGGCCAGACGCG |
| | AATTATTTTTGATGGCGTTCCTATTGGTTAAAAAATGAGCTGATTTAACAAAAATTTAATG |
| | CGAATTTTAACAAAATATTAACGTTTACAATTTAAATATTTGCTTATACAATCTTCCTGTTT |
| | TTGGGGCTTTTCTGATTATCAACCGGGGTACATATGATTGACATGCTAGTTTTACGATTA |
| | CCGTTCATCGATTCTCTTGTTTGCTCCAGACTCTCAGGCAATGACCTGATAGCCTTTGT |
| | AGATCTCTCAAAAATAGCTACCCTCTCCGGCATTAATTTATCAGCTAGAACGGTTGAATA |
| | TCATATTGATGGTGATTTGACTGTCTCCGGCCTTTCTCACCCTTTTGAATCTTTACCTAC |
| | ACATTACTCAGGCATTGCATTTAAAATATATGAGGGTTCTAAAAATTTTTATCCTTGCGTT |
| | GAAATAAAGGCTTCTCCCGCAAAAGTATTACAGGGTCATAATGTTTTTGGTACAACCGA |
| | TTTAGCTTTATGCTCTGAGGCTTTATTGCTTAATTTTGCTAATTCTTTGCCTTGCCTGTAT |
| | GATTTATTGGATGTT |
| PhiX174 | GAGTTTTATCGCTTCCATGACGCAGAAGTTAACACTTTCGGATATTTCTGATGAGTCGA |
| (SEQ ID NO: 14) | AAAATTATCTTGATAAAGCAGGAATTACTACTGCTTGTTTACGAATTAAATCGAAGTGGA |
| | CTGCTGGCGGAAAATGAGAAAATTCGACCTATCCTTGCGCAGCTCGAGAAGCTCTTAC |
| | TTTGCGACCTTTCGCCATCAACTAACGATTCTGTCAAAAACTGACGCGTTGGATGAGGA |
| | GAAGTGGCTTAATATGCTTGGCACGTTCGTCAAGGACTGGTTTAGATATGAGTCACATT |
| | TTGTTCATGGTAGAGATTCTCTTGTTGACATTTTAAAAGAGCGTGGATTACTATCTGAGT |
| | CCGATGCTGTTCAACCACTAATAGGTAAGAAATCATGAGTCAAGTTACTGAACAATCCG |
| | TACGTTTCCAGACCGCTTTGGCCTCTATTAAGCTCATTCAGGCTTCTGCCGTTTTGGAT |
| | TTAACCGAAGATGATTTCGATTTTCTGACGAGTAACAAAGTTTGGATTGCTACTGACCG |
| | CTCTCGTGCTCGTCGCTGCGTTGAGGCTTGCGTTTATGGTACGCTGGACTTTGTAGGA |
| | TACCCTCGCTTTCCTGCTCCTGTTGAGTTTATTGCTGCCGTCATTGCTTATTATGTTCAT |
| | CCCGTCAACATTCAAACGGCCTGTCTCATCATGGAAGGCGCTGAATTTACGGAAAACAT |
| | TATTAATGGCGTCGAGCGTCCGGTTAAAGCCGCTGAATTGTTCGCGTTTACCTTGCGT |
| | GTACGCGCAGGAAACACTGACGTTCTTACTGACGCAGAAGAAAACGTGCGTCAAAAAT |
| | TACGTGCAGAAGGAGTGATGTAATGTCTAAAGGTAAAAAACGTTCTGGCGCTCGCCCT |
| | GGTCGTCCGCAGCCGTTGCGAGGTACTAAAGGCAAGCGTAAAGGCGCTCGTCTTTGG |
| | TATGTAGGTGGTCAACAATTTTAATTGCAGGGGCTTCGGCCCCTTACTTGAGGATAAAT |
| | TATGTCTAATATTCAAACTGGCGCCGAGCGTATGCCGCATGACCTTTCCCATCTTGGCT |
| | TCCTTGCTGGTCAGATTGGTCGTCTTATTACCATTTCAACTACTCCGGTTATCGCTGGC |
| | GACTCCTTCGAGATGGACGCCGTTGGCGCTCTCCGTCTTTCTCCATTGCGTCGTGGCC |
| | TTGCTATTGACTCTACTGTAGACATTTTTACTTTTTATGTCCCTCATCGTCACGTTTATGG |
| | TGAACAGTGGATTAAGTTCATGAAGGATGGTGTTAATGCCACTCCTCTCCCGACTGTTA |
| | ACACTACTGGTTATATTGACCATGCCGCTTTTCTTGGCACGATTAACCCTGATACCAATA |
| | AAATCCCTAAGCATTTGTTTCAGGGTTATTTGAATATCTATAACAACTATTTTAAAGCGC |
| | CGTGGATGCCTGACCGTACCGAGGCTAACCCTAATGAGCTTAATCAAGATGATGCTCG |
| | TTATGGTTTCCGTTGCTGCCATCTCAAAAACATTTGGACTGCTCCGCTTCCTCCTGAGA |
| | CTGAGCTTTCTCGCCAAATGACGACTTCTACCACATCTATTGACATTATGGGTCTGCAA |
| | GCTGCTTATGCTAATTTGCATACTGACCAAGAACGTGATTACTTCATGCAGCGTTACCA |
| | TGATGTTATTTCTTCATTTGGAGGTAAAACCTCTTATGACGCTGACAACCGTCCTTTACT |
| | TGTCATGCGCTCTAATCTCTGGGCATCTGGCTATGATGTTGATGGAACTGACCAAACGT |
| | CGTTAGGCCAGTTTTCTGGTCGTGTTCAACAGACCTATAAACATTCTGTGCCGCGTTTC |
| | TTTGTTCCTGAGCATGGCACTATGTTTACTCTTGCGCTTGTTCGTTTTCCGCCTACTGC |
| | GACTAAAGAGATTCAGTACCTTAACGCTAAAGGTGCTTTGACTTATACCGATATTGCTG |
| | GCGACCCTGTTTTGTATGGCAACTTGCCGCCGCGTGAAATTTCTATGAAGGATGTTTTC |
| | CGTTCTGGTGATTCGTCTAAGAAGTTTAAGATTGCTGAGGGTCAGTGGTATCGTTATGC |
| | GCCTTCGTATGTTTCTCCTGCTTATCACCTTCTTGAAGGCTTCCCATTCATTCAGGAACC |
| | GCCTTCTGGTGATTTGCAAGAACGCGTACTTATTCGCCACCATGATTATGACCAGTGTT |
| | TCCAGTCCGTTCAGTTGTTGCAGTGGAATAGTCAGGTTAAATTTAATGTGACCGTTTAT |
| | CGCAATCTGCCGACCACTCGCGATTCAATCATGACTTCGTGATAAAAGATTGAGTGTGA |
| | GGTTATAACGCCGAAGCGGTAAAAATTTTAATTTTTGCCGCTGAGGGGTTGACCAAGC |
| | GAAGCGCGGTAGGTTTTCTGCTTAGGAGTTTAATCATGTTTCAGACTTTTATTTCTCGCC |
| | ATAATTCAAACTTTTTTTCTGATAAGCTGGTTCTCACTTCTGTTACTCCAGCTTCTTCGG |
| | CACCTGTTTTACAGACACCTAAAGCTACATCGTCAACGTTATATTTTGATAGTTTGACGG |
| | TTAATGCTGGTAATGGTGGTTTTCTTCATTGCATTCAGATGGATACATCTGTCAACGCC |
| | GCTAATCAGGTTGTTTCTGTTGGTGCTGATATTGCTTTTGATGCCGACCCTAAATTTTTT |
| | GCCTGTTTGGTTCGCTTTGAGTCTTCTTCGGTTCCGACTACCCTCCCGACTGCCTATGA |
| | TGTTTATCCTTTGGATGGTCGCCATGATGGTGGTTATTATACCGTCAAGGACTGTGTGA |
| | CTATTGACGTCCTTCCCCGTACGCCGGGCAATAATGTTTATGTTGGTTTCATGGTTTGG |
| | TCTAACTTTACCGCTACTAAATGCCGCGGATTGGTTTCGCTGAATCAGGTTATTAAAGA |
| | GATTATTTGTCTCCAGCCACTTAAGTGAGGTGATTTATGTTTGGTGCTATTGCTGGCGG |
| | TATTGCTTCTGCTCTTGCTGGTGGCGCCATGTCTAAATTGTTTGGAGGCGGTCAAAAAG |
| | CCGCCTCCGGTGGCATTCAAGGTGATGTGCTTGCTACCGATAACAATACTGTAGGCAT |
| | GGGTGAGAGTTTTATCGCTTCCATGACGCAGAAGTTAACACTTTCGGATATTTCTGATG |
| | AGTCGAAAAATTATCTTGATAAAGCAGGAATTACTACTGCTTGTTTACGAATTAAATCGA |
| | AGTGGACTGCTGGCGGAAAATGAGAAAATTCGACCTATCCTTGCGCAGCTCGAGAAGC |
| | TCTTACTTTGCGACCTTTCGCCATCAACTAACGATTCTGTCAAAAACTGACGCGTTGGA |
| | TGAGGAGAAGTGGCTTAATATGCTTGGCACGTTCGTCAAGGACTGGTTTAGATATGAGT |
| | CACATTTTGTTCATGGTAGAGATTCTCTTGTTGACATTTTAAAAGAGCGTGGATTACTAT |
| | CTGAGTCCGATGCTGTTCAACCACTAATAGGTAAGAAATCATGAGTCAAGTTACTGAAC |
| | AATCCGTACGTTTCCAGACCGCTTTGGCCTCTATTAAGCTCATTCAGGCTTCTGCCGTT |
| | TTGGATTTAACCGAAGATGATTTCGATTTTCTGACGAGTAACAAAGTTTGGATTGCTACT |
| | GACCGCTCTCGTGCTCGTCGCTGCGTTGAGGCTTGCGTTTATGGTACGCTGGACTTTG |
| | TAGGATACCCTCGCTTTCCTGCTCCTGTTGAGTTTATTGCTGCCGTCATTGCTTATTATG |
| | TTCATCCCGTCAACATTCAAACGGCCTGTCTCATCATGGAAGGCGCTGAATTTACGGAA |
| | AACATTATTAATGGCGTCGAGCGTCCGGTTAAAGCCGCTGAATTGTTCGCGTTTACCTT |
| | GCGTGTACGCGCAGGAAACACTGACGTTCTTACTGACGCAGAAGAAAACGTGCGTCAA |
| | AAATTACGTGCAGAAGGAGTGATGTAATGTCTAAAGGTAAAAAACGTTCTGGCGCTCGC |
| | CCTGGTCGTCCGCAGCCGTTGCGAGGTACTAAAGGCAAGCGTAAAGGCGCTCGTCTTT |
| | GGTATGTAGGTGGTCAACAATTTTAATTGCAGGGGCTTCGGCCCCTTACTTGAGGATAA |
| | ATTATGTCTAATATTCAAACTGGCGCCGAGCGTATGCCGCATGACCTTTCCCATCTTGG |
| | CTTCCTTGCTGGTCAGATTGGTCGTCTTATTACCATTTCAACTACTCCGGTTATCGCTG |
| | GCGACTCCTTCGAGATGGACGCCGTTGGCGCTCTCCGTCTTTCTCCATTGCGTCGTGG |
| | CCTTGCTATTGACTCTACTGTAGACATTTTTACTTTTTATGTCCCTCATCGTCACGTTTAT |
| | GGTGAACAGTGGATTAAGTTCATGAAGGATGGTGTTAATGCCACTCCTCTCCCGACTGT |
| | TAACACTACTGGTTATATTGACCATGCCGCTTTTCTTGGCACGATTAACCCTGATACCAA |
| | TAAAATCCCTAAGCATTTGTTTCAGGGTTATTTGAATATCTATAACAACTATTTTAAAGCG |
| | CCGTGGATGCCTGACCGTACCGAGGCTAACCCTAATGAGCTTAATCAAGATGATGCTC |
| | GTTATGGTTTCCGTTGCTGCCATCTCAAAAACATTTGGACTGCTCCGCTTCCTCCTGAG |
| | ACTGAGCTTTCTCGCCAAATGACGACTTCTACCACATCTATTGACATTATGGGTCTGCA |
| | AGCTGCTTATGCTAATTTGCATACTGACCAAGAACGTGATTACTTCATGCAGCGTTACC |
| | ATGATGTTATTTCTTCATTTGGAGGTAAAACCTCTTATGACGCTGACAACCGTCCTTTAC |
| | TTGTCATGCGCTCTAATCTCTGGGCATCTGGCTATGATGTTGATGGAACTGACCAAACG |
| | TCGTTAGGCCAGTTTTCTGGTCGTGTTCAACAGACCTATAAACATTCTGTGCCGCGTTT |
| | CTTTGTTCCTGAGCATGGCACTATGTTTACTCTTGCGCTTGTTCGTTTTCCGCCTACTG |
| | CGACTAAAGAGATTCAGTACCTTAACGCTAAAGGTGCTTTGACTTATACCGATATTGCT |
| | GGCGACCCTGTTTTGTATGGCAACTTGCCGCCGCGTGAAATTTCTATGAAGGATGTTTT |
| | CCGTTCTGGTGATTCGTCTAAGAAGTTTAAGATTGCTGAGGGTCAGTGGTATCGTTATG |
| | CGCCTTCGTATGTTTCTCCTGCTTATCACCTTCTTGAAGGCTTCCCATTCATTCAGGAAC |
| | CGCCTTCTGGTGATTTGCAAGAACGCGTACTTATTCGCCACCATGATTATGACCAGTGT |
| | TTCCAGTCCGTTCAGTTGTTGCAGTGGAATAGTCAGGTTAAATTTAATGTGACCGTTTAT |
| | CGCAATCTGCCGACCACTCGCGATTCAATCATGACTTCGTGATAAAAGATTGAGTGTGA |
| | GGTTATAACGCCGAAGCGGTAAAAATTTTAATTTTTGCCGCTGAGGGGTTGACCAAGC |
| | GAAGCGCGGTAGGTTTTCTGCTTAGGAGTTTAATCATGTTTCAGACTTTTATTTCTCGCC |
| | ATAATTCAAACTTTTTTTCTGATAAGCTGGTTCTCACTTCTGTTACTCCAGCTTCTTCGG |
| | CACCTGTTTTACAGACACCTAAAGCTACATCGTCAACGTTATATTTTGATAGTTTGACGG |
| | TTAATGCTGGTAATGGTGGTTTTCTTCATTGCATTCAGATGGATACATCTGTCAACGCC |
| | GCTAATCAGGTTGTTTCTGTTGGTGCTGATATTGCTTTTGATGCCGACCCTAAATTTTTT |
| | GCCTGTTTGGTTCGCTTTGAGTCTTCTTCGGTTCCGACTACCCTCCCGACTGCCTATGA |
| | TGTTTATCCTTTGGATGGTCGCCATGATGGTGGTTATTATACCGTCAAGGACTGTGTGA |
| | CTATTGACGTCCTTCCCCGTACGCCGGGCAATAATGTTTATGTTGGTTTCATGGTTTGG |
| | TCTAACTTTACCGCTACTAAATGCCGCGGATTGGTTTCGCTGAATCAGGTTATTAAAGA |
| | GATTATTTGTCTCCAGCCACTTAAGTGAGGTGATTTATGTTTGGTGCTATTGCTGGCGG |
| | TATTGCTTCTGCTCTTGCTGGTGGCGCCATGTCTAAATTGTTTGGAGGCGGTCAAAAAG |
| | CCGCCTCCGGTGGCATTCAAGGTGATGTGCTTGCTACCGATAACAATACTGTAGGCAT |
| | GGGTGATGCTGGTATTAAATCTGCCATTCAAGGCTCTAATGTTCCTAACCCTGATGAGG |
| | CCGTCCCTAGTTTTGTTTCTGGTGCTATGGCTAAAGCTGGTAAAGGACTTCTTGAAGGT |
| | ACGTTGCAGGCTGGCACTTCTGCCGTTTCTGATAAGTTGCTTGATTTGGTTGGACTTGG |
| | TGGCAAGTCTGCCGCTGATAAAGGAAAGGATACTCGTGATTATCTTGCTGCTGCATTTC |
| | CTGAGCTTAATGCTTGGGAGCGTGCTGGTGCTGATGCTTCCTCTGCTGGTATGGTTGA |
| | CGCCGGATTTGAGAATCAAAAAGAGCTTACTAAAATGCAACTGGACAATCAGAAAGAGA |
| | TTGCCGAGATGCAAAATGAGACTCAAAAAGAGATTGCTGGCATTCAGTCGGCGACTTC |
| | ACGCCAGAATACGAAAGACCAGGTATATGCACAAAATGAGATGCTTGCTTATCAACAGA |
| | AGGAGTCTACTGCTCGCGTTGCGTCTATTATGGAAAACACCAATCTTTCCAAGCAACAG |
| | CAGGTTTCCGAGATTATGCGCCAAATGCTTACTCAAGCTCAAACGGCTGGTCAGTATTT |
| | TACCAATGACCAAATCAAAGAAATGACTCGCAAGGTTAGTGCTGAGGTTGACTTAGTTC |
| | ATCAGCAAACGCAGAATCAGCGGTATGGCTCTTCTCATATTGGCGCTACTGCAAAGGA |
| | TATTTCTAATGTCGTCACTGATGCTGCTTCTGGTGTGGTTGATATTTTTCATGGTATTGA |
| | TAAAGCTGTTGCCGATACTTGGAACAATTTCTGGAAAGACGGTAAAGCTGATGGTATTG |
| | GCTCTAATTTGTCTAGGAAATAACCGTCAGGATTGACACCCTCCCAATTGTATGTTTTCA |
| | TGCCTCCAAATCTTGGAGGCTTTTTTATGGTTCGTTCTTATTACCCTTCTGAATGTCACG |
| | CTGATTATTTTGACTTTGAGCGTATCGAGGCTCTTAAACCTGCTATTGAGGCTTGTGGC |
| | ATTTCTACTCTTTCTCAATCCCCAATGCTTGGCTTCCATAAGCAGATGGATAACCGCATC |
| | AAGCTCTTGGAAGAGATTCTGTCTTTTCGTATGCAGGGCGTTGAGTTCGATAATGGTGA |
| | TATGTATGTTGACGGCCATAAGGCTGCTTCTGACGTTCGTGATGAGTTTGTATCTGTTA |
| | CTGAGAAGTTAATGGATGAATTGGCACAATGCTACAATGTGCTCCCCCAACTTGATATT |
| | AATAACACTATAGACCACCGCCCCGAAGGGGACGAAAAATGGTTTTTAGAGAACGAGA |
| | AGACGGTTACGCAGTTTTGCCGCAAGCTGGCTGCTGAACGCCCTCTTAAGGATATTCG |
| | CGATGAGTATAATTACCCCAAAAAGAAAGGTATTAAGGATGAGTGTTCAAGATTGCTGG |
| | AGGCCTCCACTATGAAATCGCGTAGAGGCTTTGCTATTCAGCGTTTGATGAATGCAATG |
| | CGACAGGCTCATGCTGATGGTTGGTTTATCGTTTTTGACACTCTCACGTTGGCTGACGA |
| | CCGATTAGAGGCGTTTTATGATAATCCCAATGCTTTGCGTGACTATTTTCGTGATATTGG |
| | TCGTATGGTTCTTGCTGCCGAGGGTCGCAAGGCTAATGATTCACACGCCGACTGCTAT |
| | CAGTATTTTTGTGTGCCTGAGTATGGTACAGCTAATGGCCGTCTTCATTTCCATGCGGT |
| | GCACTTTATGCGGACACTTCCTACAGGTAGCGTTGACCCTAATTTTGGTCGTCGGGTAC |
| | GCAATCGCCGCCAGTTAAATAGCTTGCAAAATACGTGGCCTTATGGTTACAGTATGCCC |
| | ATCGCAGTTCGCTACACGCAGGACGCTTTTTCACGTTCTGGTTGGTTGTGGCCTGTTG |
| | ATGCTAAAGGTGAGCCGCTTAAAGCTACCAGTTATATGGCTGTTGGTTTCTATGTGGCT |
| | AAATACGTTAACAAAAAGTCAGATATGGACCTTGCTGCTAAAGGTCTAGGAGCTAAAGA |
| | ATGGAACAACTCACTAAAAACCAAGCTGTCGCTACTTCCCAAGAAGCTGTTCAGAATCA |
| | GAATGAGCCGCAACTTCGGGATGAAAATGCTCACAATGACAAATCTGTCCACGGAGTG |
| | CTTAATCCAACTTACCAAGCTGGGTTACGACGCGACGCCGTTCAACCAGATATTGAAG |
| | CAGAACGCAAAAAGAGAGATGAGATTGAGGCTGGGAAAAGTTACTGTAGCCGACGTTT |
| | TGGCGGCGCAACCTGTGACGACAAATCTGCTCAAATTTATGCGCGCTTCGATAAAAAT |
| | GATTGGCGTATCCAACCTGCA |

TABLE 4
Staple strands for nucleic acid polygonal nanopore:
Staple sequences for the polygonal frames (folded with M13 scaffold) and
other nanopores (folded with PhiX174 scaffold).

| | SEQ ID | |
|---|---|---|
| Name | NO. | Sequence |
| 10nmTriangle01 | 15 | AGTAAGCAGATAGCTCAGAGCAGGCGAACAA |
| 10nmTriangle02 | 16 | GAAATAGCTAATAACGGAAGAGCCCACCATACCC |
| 10nmTriangle03 | 17 | AGTTTACATACATAAAGGTGGCAACATAAGAACCACGCCGCCAG |
| 10nmTriangle04 | 18 | AAACGCAAAATAGCTAAGAAA |
| 10nmTriangle05 | 19 | AGGTTGAGCGATTGGCAAATCCTCATAACAAT |
| 10nmTriangle06 | 20 | AAAAGAACTGGCATGTAGAAAAACCAGAAG |
| 10nmTriangle07 | 21 | TAGCGTTTCCCTCAGAGCCTAATCCACCGACCAC |
| 10nmTriangle08 | 22 | CCCTGCAAAGACACCACGGAATAAGTTTGATAGCAGAGTAGCGA |
| 10nmTriangle09 | 23 | CCTCAGAGCCGCCAAAAGAAACCAGAGCCG |
| 10nmTriangle10 | 24 | GAGCCACCACCGGATCAGATAGCGACCGCCT |
| 10nmTriangle11 | 25 | AACCGCCAGCCATCTTAACCA |
| 10nmTriangle12 | 26 | TTTGCCTTCTGTAGCGGTCATAGCCCCCTTAT |
| 10nmTriangle13 | 27 | ATTCTGAAACCATTTGTCACATTAGCAA |
| 10nmTriangle14 | 28 | TACCAATCAATACGCAGTATGTTAGCAATTAAGACTCCAAAGAC |
| 10nmTriangle15 | 29 | GTCACCAACACCGACTGCCAG |
| 10nmTriangle16 | 30 | CATTCAACAGGGAAGGTCATTAAAGGTGAATTATC |
| 10nmTriangle17 | 31 | CAAAATCACCAGTAGAGGGCGATTGCACCAT |
| 10nmTriangle18 | 32 | ACCGTATATGGTTTACCAGCGCCTTATTAGAAA |
| 10nmSquare01 | 33 | GACTTATAGCCCCCACAGACAGCCCTCACAACGCCTGGAGGTTT |
| 10nmSquare02 | 34 | CCCTCAGATCAGAACCGAACCGAACTTTTGAT |
| 10nmSquare03 | 35 | GATTAGCGAGTGTACTGAAAC |
| 10nmSquare04 | 36 | GATACAGGTGTATCACCGTACTCAGTAGCATTGGAA |
| 10nmSquare05 | 37 | ATGAAAGTATTAAGCACCCACCGCAGGCTGA |
| 10nmSquare06 | 38 | TAGGGGGTTTTGGATATAAGCCTCAAGA |
| 10nmSquare07 | 39 | TTTTGTACAAACGTTAGCGTTACCGTAA |
| 10nmSquare08 | 40 | ATAGCAAGCCCAATCTAAATTTTGAGGAACC |
| 10nmSquare09 | 41 | CCATGTTAGTCGTCTTTCCAGACGTTTAATTGAAAG |
| 10nmSquare10 | 42 | CATGAACGATCTTATCGGTTTATCAGCTAAGGAGCCTTAGTAAA |
| 10nmSquare11 | 43 | CGTCACCACTTAGCCGCAGGG |
| 10nmSquare12 | 44 | GTATGGGACAACTTTCAAATCCGCGACCTGCT |
| 10nmSquare13 | 45 | GCCACTACGTTAATGCTGAGTAACAGTGCCCGAGGC |
| 10nmSquare14 | 46 | CTCAGCAGGAATACACAAGGCCGCAACG |
| 10nmSquare15 | 47 | AAAAAGGAAGTTTCCATTAAGGCGGATATTTT |
| 10nmSquare16 | 48 | CTAATAAAGACTAGTGCCGTCGAGAGGGCAGTACCAACGGGTAA |
| 10nmSquare17 | 49 | AAAGTATAAACAGAAGGCACCAAC |
| 10nmSquare18 | 50 | CATGCGAAAGACTTGAGGACTTTTGCGG |
| 10nmSquare19 | 51 | AATTGTGAATTTGCAACGGCTACAGAGGCATCGGAATGCGCCGA |
| 10nmSquare20 | 52 | AACCATCGACCGATATTATACCAAGCGCGAAA |
| 10nmSquare21 | 53 | AACAAAGGCTCCCTTTCGAGTTTTCACG |
| 10nmSquare22 | 54 | CCAAAAAAAACGGAGAGAACA |
| 10nmSquare23 | 55 | ACTAAAGGAATTGCGCATACCCACGAATAAT |
| 10nmSquare24 | 56 | CAAAGTACGCTTGATACCGATAGTCGAGGGTACTTA |
| 10nmSquare- | 57 | TGTATCATCGCCTGATAAATTGTGT |
| spacer01 | | |
| 10nmSquare- | 58 | AAACACTCATCTTTGACCCCCAGCG |
| spacer02 | | |
| 10nmSquare- | 59 | CACCCTCAGAGCCACCACCCTCATT |
| spacer03 | | |
| 10nmSquare- | 60 | CCTGCCTATTTCGGAACCTATTATT |
| spacer04 | | |
| 10nmSquare- | 61 | CAGTTTCAGCGGAGTGAGAATAGAA |
| spacer05 | | |
| 10nmSquare- | 62 | TAATAAGTTTTAACGGGGTCAGTGC |
| spacer06 | | |
| 10nmSquare- | 63 | ACGAGGCGCAGACGGTCAATCATAA |
| spacer07 | | |
| 10nmSquare- | 64 | TCGGTCGCTGAGGCTTGCAGGGAGT |
| spacer08 | | |
| 10nmPentagon01 | 65 | CTTGCCCTACTAATGCAGATACATATAGCGTCCACA |
| 10nmPentagon02 | 66 | AATAAAACGACGAGAAAACTG |
| 10nmPentagon03 | 67 | TTCAGAACTAACTGAGATTTGGAAGAAA |
| 10nmPentagon04 | 68 | GAGGCATACACTATCAATATGTACCCATAAGG |
| 10nmPentagon05 | 69 | GTTGAGGAATACCAATACTGCGGAATCGTAGACTGGAACGCCAA |
| 10nmPentagon06 | 70 | GCTCATTATACCAGAGCAAGTAAGTCAGGAC |
| 10nmPentagon07 | 71 | GAGAATCGAAATGCTTTAAACAGTAACCAGACTCCC |
| 10nmPentagon08 | 72 | CATAAATCTCTTTACCAGTCTGGAGCAAACAA |
| 10nmPentagon09 | 73 | CCAAAATAGCGAGATCAGGAAAAAGGCTTTT |
| 10nmPentagon10 | 74 | CCTCGTAAAATGATAAATATAAGAAGTT |
| 10nmPentagon11 | 75 | GCAATCATTGAACGGAAGCAAACTCCAATCAAAGCGTCAGAAAA |
| 10nmPentagon12 | 76 | GGGTAATAATGAACGGAAAAA |
| 10nmPentagon13 | 77 | GGAGAGCCTCAGGACCCTGTAAAGAATT |
| 10nmPentagon14 | 78 | ATGTAAGCCTTTATTTCAACTATTACAGTGCG |
| 10nmPentagon15 | 79 | ATGCAATACTTTGTAGAAAGATTCATCAAACAACATGCAAGGAT |
| 10nmPentagon16 | 80 | AGCAATAAGTAGGTAAGGCAAGGCAATG |
| 10nmPentagon17 | 81 | AGAACCCTTAATCATTCTTGAGATGGTTTAATAGTA |
| 10nmPentagon18 | 82 | CCTGTTCAACTTCATATATTTTAA |
| 10nmPentagon19 | 83 | TGATTCCCTGATATTCATATG |
| 10nmPentagon20 | 84 | TGGCATCATAGTAGTAGAGACAGTCAAATCAC |
| 10nmPentagon21 | 85 | AAGTGTCAATAATTGTACCAAAAACATTCATAAAGCGGGCGCGA |
| 10nmPentagon22 | 86 | CAACTAAAGTACGGACTAAATTCTTGTCTGG |
| 10nmPentagon23 | 87 | TTTAAATTCTGCCGCAAATGTTCATTCC |
| 10nmPentagon24 | 88 | CATCAATAGCTATATTTTCATTTGTAAATCGGCCTG |
| 10nmPentagon25 | 89 | TTTTTGAGATTGCTCCTTTTGATAATTTAGTTTACC |
| 10nmPentagon26 | 90 | TTTAAATTCGAGGGTCAGGACCGAAAGA |
| 10nmPentagon27 | 91 | TGCATCAAAAAGATAATTGAGCTTTAAGAGG |
| 10nmPentagon28 | 92 | AAGCTTAGAGAGTGACCATTAGATACATACGAGTAGAGAGGTCA |
| 10nmPentagon29 | 93 | TGGCTTAGCTGAATATCCGGAGAGGGTAGCTA |
| 10nmPentagon30 | 94 | CGCGTTTTAGATCTACCGGAT |
| 10nmPentagon- | 95 | ACCAGAACGAGTAGTAAATTG |
| spacer01 | | |
| 10nmPentagon- | 96 | GACTATTATAGTCAGAAGCAA |
| spacer02 | | |
| 10nmPentagon- | 97 | ATCGTAAAACTAGCATGTCAA |
| spacer03 | | |
| 10nmPentagon- | 98 | TGCTGTAGCTCAACATGTTTT |
| spacer04 | | |
| 10nmPentagon- | 99 | GAATTACCTTATGCGATTTTA |
| spacer05 | | |
| 10nmPentagon- | 100 | ACCCTCGTTTACCAGACGACG |
| spacer06 | | |
| 10nmPentagon- | 101 | ATTCAAAAGGGTGAGAAAGGC |
| spacer07 | | |
| 10nmPentagon- | 102 | ATTAACATCCAATAAATCATA |
| spacer08 | | |
| 10nmPentagon- | 103 | AGGCTATCAGGTCATTGCCTG |
| spacer09 | | |
| 10nmPentagon- | 104 | CCGTTCTAGCTGATAAATTAA |
| spacer10 | | |
| 10nmHexagon01 | 105 | AATAGCGACAAAAGAACCGTGCATCTAACTTT |
| 10nmHexagon02 | 106 | AATCATTGTAACCCTCGTTTACCATTAAACAGCACT |
| 10nmHexagon03 | 107 | AAAGAAGAGCAATTCAGAAAACGAGAATCAAATGCTGACGACGA |
| 10nmHexagon04 | 108 | ATCACACATTCAGGCATAGTATTCATCA |
| 10nmHexagon05 | 109 | AGGAATACTGAATTACTAACG |
| 10nmHexagon06 | 110 | GAACAACATTATTATTTTGGAGGCCAGGTAG |
| 10nmHexagon07 | 111 | TATTCATTGTCACGTTTAAAA |
| 10nmHexagon08 | 112 | TGGGATAGACCCTGACTATTATAGCTCCTTTTAGGT |
| 10nmHexagon09 | 113 | AATACAAAAATCGATAAGAGGTCATTTTTTTAATTGTCAGAAGC |
| 10nmHexagon10 | 114 | CTTTGAATCCCCCCATAAATCTGCGGAA |
| 10nmHexagon11 | 115 | GCATCAAAGGAAGCCCGGCGGATTGACCGTAA |
| 10nmHexagon12 | 116 | TGTTTAGACTGGATTAAGAAAGATAGCGTCC |
| 10nmHexagon13 | 117 | TTATATGCAATGTTGCCTGAGAGTCTGGAAAGGCTATCAAAAGG |
| 10nmHexagon14 | 118 | CCGGAGACCCATCAATTATTTTGTTAAAATTC |
| 10nmHexagon15 | 119 | AGTAGCCTTTATTATTTTAAGACCCTGT |
| 10nmHexagon16 | 120 | AAATCGGTTGTACCAATCAAGTCAAAAAACA |
| 10nmHexagon17 | 121 | CGGGAGAATTTTTGTTAAGCT |
| 10nmHexagon18 | 122 | GCATTAAAATGTGTAGGTAAAGATTCAGGTCACCTG |
| 10nmHexagon19 | 123 | CAGGGGACGTTGAATCAGAAAAGC |
| 10nmHexagon20 | 124 | CGGTTGATGGAAGAAACTCATTATACCAGTCAAAGA |
| 10nmHexagon21 | 125 | CCCAAGAATCGACGCCAAAAGGAATTACTAATGCAGTGTCAATC |
| 10nmHexagon22 | 126 | TATTTTTGATAAGCAAGATAAATTAAAA |
| 10nmHexagon23 | 127 | CGGTAGAGATCTCAAACAAGAATGCCGG |
| 10nmHexagon24 | 128 | TTGTAATCGTAAAACTAGCAATACATAATGAA |
| 10nmHexagon25 | 129 | ACTAGGTCAATAGAAAAGGTTTGACCAT |
| 10nmHexagon26 | 130 | GGCAAGGCCAAAATTAATAGGAACGCCATCAA |
| 10nmHexagon27 | 131 | AAATAATTATAGTAGTAGCATTAAGGATAAAATTCT |
| 10nmHexagon28 | 132 | AATTCTGCGAACGAATTAGAAAGAGTAGATT |
| 10nmHexagon29 | 133 | TCGCAAATCGCGTCTGTTCCC |
| 10nmHexagon30 | 134 | TAGTGGCATCAAATTTTTAGAACCCTCACAACGCAACATCCAAT |
| 10nmHexagon31 | 135 | TGCTAGAGAGTACGGATGGCGAAGCAAA |
| 10nmHexagon32 | 136 | ACCGTTAGAGCTTCATTTGGGGCGCGAGCTGTTTAGCTCAACAT |
| 10nmHexagon33 | 137 | TCAGGATTCAACCCGTTTAAT |
| 10nmHexagon34 | 138 | TCGAGCTTCAAAGCCGGTGAAGTAGAACCAG |
| 10nmHexagon35 | 139 | TGCAACTATCTGGAAGTCAACATTAAATGTGA |
| 10nmHexagon36 | 140 | GCGAGTAAGAATATAATGCTGTAGCTATATTTTAAT |
| 10nmHexagon- | 141 | ATTTAAATTGTAAACGTT |
| spacer01 | | |
| 10nmHexagon- | 142 | TCATTCCATATAACAGTT |
| spacer02 | | |
| 10nmHexagon- | 143 | TGTAGATGGGCGCATCGT |
| spacer03 | | |
| 10nmHexagon- | 144 | GATTCTCCGTGGGAACAA |
| spacer04 | | |
| 10nmHexagon- | 145 | ATCAGCTCATTTTTTAAC |
| spacer05 | | |
| 10nmHexagon- | 146 | CTTCCTGTAGCCAGCTTT |
| spacer06 | | |
| 10nmHexagon- | 147 | TCTACGTTAATAAAACGA |
| spacer07 | | |
| 10nmHexagon- | 148 | TTTGCCAGAGGGGGTAAT |
| spacer08 | | |
| 10nmHexagon- | 149 | AAGACTTCAAATATCGCG |
| spacer09 | | |
| 10nmHexagon- | 150 | TATGCGATTTTAAGAACT |
| spacer10 | | |
| 10nmHexagon- | 151 | CAATAAAGCCTCAGAGCA |
| spacer11 | | |
| 10nmHexagon- | 152 | GATATTCAACCGTTCTAG |
| spacer12 | | |
| 20nmTriangle01 | 153 | GTAGTCGTCGCTTAGCTTAGTGAGAAGAG |
| 20nmTriangle02 | 154 | CGTTAGAACGCGAAAAAAGCCTGTGCTAAGTTCATTAG |
| 20nmTriangle03 | 155 | AATAGTTTAACGTCAGATGAACCATATCATCATTTTATAT |
| 20nmTriangle04 | 156 | ATTATTTGCACGTAAAACAGATTGC |
| 20nmTriangle05 | 157 | AATAAATGATGAAACAAATTAATTACATTTAACAATTTCACTT |
| 20nmTriangle06 | 158 | CTTGAAAACGAATTATAGCAAAAGAAGAGAAA |
| 20nmTriangle07 | 159 | CGACAATAAACAACATTGCAGAACTGTTTTGA |
| 20nmTriangle08 | 160 | AAAAGAAAATTTTCAGCCAAGTTACAAAA |
| 20nmTriangle09 | 161 | GAACAATTACTAGGAAAACTTTTTCAAATAATAGATTCAATTCGA |
| 20nmTriangle10 | 162 | GAGCGGAATTGTTTGGGGAAGGGTTAGAACCTATA |
| 20nmTriangle11 | 163 | AACCATTAAATCTAACAGTAACAATAACGGAT |
| 20nmTriangle12 | 164 | TCGCGCAGAGGCATAGCGAATT |
| 20nmTriangle13 | 165 | TCGCCTGATTGCTATTAGACGAA |
| 20nmTriangle14 | 166 | GCAATTCATCAATATAATCCTGATTATCATCGCG |
| 20nmTriangle15 | 167 | ATAATACATTAAGACAAATTAATTTTGTAACATTAAA |
| 20nmTriangle16 | 168 | ATTAAGGATTTAGAAGTTTG |
| 20nmTriangle17 | 169 | TACAGCTTTGCCCTTTACAAAAGAGCCGTCAATAG |
| 20nmTriangle18 | 170 | TGAGTGAATAACATGGAAACCCAGACGA |
| 20nmTriangle19 | 171 | TCAATAGTGAATTTACTATATGTATTTTCCCTATG |
| 20nmTriangle20 | 172 | AATTAAAATGCTGTTATCAACAATAGATAAATTCTGTAGTACATA |
| 20nmTriangle21 | 173 | TTTTACTTGCTTCGCGCCTGTATGCAAATCCAATCGCTGAGACGC |
| 20nmTriangle22 | 174 | TCCTGATTATCAGATATTACTTTGAGATG |
| 20nmTriangle23 | 175 | TATCATATGCGTTATACAAATTCGAATCATAAAGA |
| 20nmTriangle24 | 176 | TTGCTCACTTGCCTATCATTCC |
| 20nmTriangle25 | 177 | TAGAGTCTGTCCCTTCTGACACGTGGCACAGA |
| 20nmTriangle26 | 178 | AATAAAACCCTCAATTGAGGAAAATATCT |
| 20nmTriangle27 | 179 | TTAGGAGCACTAACATTTTAGTTTCTGGTCAAAAG |
| 20nmTriangle28 | 180 | TTTACCCGCCAGCCATTGCAACAGGAAAAGCC |
| 20nmTriangle29 | 181 | ATATAATCGCCATATTTAACAACGCCAATCTTTCCTTGAG |
| 20nmTriangle30 | 182 | CATCACCTTGCTGAGCCAGCAAGCCAAC |
| 20nmTriangle31 | 183 | AAGGTCTGAGAAGTGTATGG |
| 20nmTriangle32 | 184 | AATGGTTTGAAACGGGTATTAAACCAAGTACCAAAGGGATGCCACCGA |
| 20nmTriangle33 | 185 | CAATAAATTTCATTAAATAAGAATAAACACCAGTATAAGCAAATG |
| 20nmTriangle34 | 186 | GCTCAACAGTAGGGCTGTGATAAAAATTTCTT |
| 20nmTriangle35 | 187 | CAATATTTTTGATTTTATAAAAT |
| 20nmTriangle36 | 188 | GCTGAGAACCTCATAAGGCGTCTTCTGACCTAAATTTATCTATCT |
| 20nmTriangle37 | 189 | AAGAATACCGACCGTTAATTGAGCCAGAA |
| 20nmTriangle38 | 190 | GTCACCCACCAGCAGAAGGCGGTCAGTATTAACACCGCCTCAC |
| 20nmTriangle39 | 191 | ATCAACAGGCCCTAAATACCGAACGAAACGAC |
| 20nmTriangle40 | 192 | AATACATCAAAGGGACTAGTCTTTAATGC |
| 20nmTriangle41 | 193 | CAATAAGTGCGCAACTTAATTGGCAGATTCACCACAGT |
| 20nmTriangle42 | 194 | TAACCGTTGTAGCTGATTAGTTTA |
| 20nmTriangle43 | 195 | GCGAACTGATATTGAAAGGAAT |
| 20nmTriangle44 | 196 | CTATATTCTGGCCAACAGAGGAAATGGAAATAACATGGTA |
| 20nmTriangle45 | 197 | GAACCATCACGCATCAGTGAGTTTAGACAGGAACGGTACGCCAGA |
| 20nmTriangle46 | 198 | AACTATCGACGCTCATTGACGCTCAATCGTCTATA |
| 20nmTriangle47 | 199 | TGAACGCTACAGCTTTCCTCGAGCGGGAG |
| 20nmTriangle48 | 200 | TTTAAGGGGAAAGCCGAGGAAGGGAAGAAAGCGAAAGGAGGGC |
| 20nmTriangle49 | 201 | CACCACACCCGCCAAGTGTAGGCAGAGG |
| 20nmTriangle50 | 202 | GTATAACAGGGAGAATTAACTGAACACCCAAGTTTTCAATAGCACGC |
| 20nmTriangle51 | 203 | GTTAGATTACCGCGCCTTGG |
| 20nmTriangle52 | 204 | GAGCACGTGAACCCTATAGAGCTTGACCGTCA |
| 20nmTriangle53 | 205 | CATTTTCGAGCCAGTATTACGAGCATGTCCCG |
| 20nmTriangle54 | 206 | ATTTATATAAAGTACCGACAAAAGGTAACCTGAACAAAATCAAGTCCG |
| 20nmTriangle55 | 207 | CTATTTTGGAGCCTAATAAACAGCCATATTATAACATAAATCTA |
| 20nmTriangle56 | 208 | GGTCCCTTTACAGAGAGAATTTATCCCAGAGGTTTTTACA |
| 20nmTriangle57 | 209 | GCACTAAATCGATAACGTGGGC |
| 20nmTriangle58 | 210 | CCTCCGGAATATCCCATCCTAATATAAGAGATATCCT |
| 20nmTriangle59 | 211 | GAACCCGCAATAGCAGGAGGTGCCGTAAA |
| 20nmTriangle60 | 212 | GCGTAGAACAAGCACCAATCAATAATCGGGTAATTTAGCGGTCAC |
| 20nmTriangle61 | 213 | AGAAAGCAAATCGGTCTGAGAGACTACCTTTTAGGACATCGTTAA |
| 20nmTriangle62 | 214 | CAAATCCAACGCTAACGAGCGTCTTTCCACACCCAGCGAA |
| 20nmTriangle63 | 215 | GAATCGCTAGCGGGCTTAAAGAAACGATTTTTTGAAAA |
| 20nmTriangle64 | 216 | GAGGCGTTTTAGCACTTGCGGATC |
| 20nmTriangle65 | 217 | GCCTTAGAAAAATCTTAGGTTGGGTTATAAAATCATAAGATATAG |
| 20nmTriangle66 | 218 | GCTGGCGCGCTTAATGTAGAAAAGCCGTTTTTATTTTATCAATCA |
| 20nmTriangle67 | 219 | CTAAACAGGAGGCCGCTCATCGACTATGGTTTAAC |
| 20nmsquare01 | 220 | TAGTACCAAAATAGCGAGAGGCTTTTGCCATAACGCTCATAAAT |
| 20nmsquare02 | 221 | CGAGTAGTAAGATTCATCAG |
| 20nmsquare03 | 222 | ATAGCGTCACCATAAAGGTAACGCCAGG |
| 20nmsquare04 | 223 | GTTTTCCCAGTCCAGAGGGGCGGA |
| 20nmsquare05 | 224 | GATAAAAAGGAAGATTTAGAAGTTTTGCACGACGTTTAAAC |
| 20nmsquare06 | 225 | ATTCATTGAAATGCTTGTAAAACGACGCAGAA |
| 20nmsquare07 | 226 | ATCGCAAAAGGAATTACGAGGCATAGTACGATAAAAAAAATGTT |
| 20nmsquare08 | 227 | TTGATACCACATAATATTTTGTTAAAATTTAAATTGCGGAACAA |
| 20nmsquare09 | 228 | AGTTCAGAAAACGAGAATGCAATACTGGTAA |
| 20nmsquare10 | 229 | CTAATAAACGTTTCAACTAATGCA |
| 20nmsquare11 | 230 | AATCTACGTTAATACCCTCAATCCAAACGAA |
| 20nmsquare12 | 231 | CAGGTAGAAAATTGGGAGAAA |
| 20nmsquare13 | 232 | ACGCCAGCCTTCAAATATCG |
| 20nmsquare14 | 233 | GAAGCTGTCGAGTTAATTAGCTCAACATATCGGTGCGACCA |
| 20nmsquare15 | 234 | CGTTCTTCAAAGCCCTCGTTTACCAGACAGCAACACAAGATTAA |
| 20nmsquare16 | 235 | CAGAAGCAAAGCGGGTAGAAACGAATTGCAT |
| 20nmsquare17 | 236 | CAACAGAGCTTAATTGCTGAATATAATGCAAACTCCGATTCCCA |
| 20nmsquare18 | 237 | CAAATATCATAACGAACCAGACCG |
| 20nmsquare19 | 238 | AGTTAACAGGTCAGGATTAGAGAGTACCGATGGCTTTAAAGTAC |
| 20nmsquare20 | 239 | GAAGTTTCGGTCAATAGCTGCGCAACTG |
| 20nmsquare21 | 240 | TTAGATACATTTCGCAAATATTCCATATATG |
| 20nmsquare22 | 241 | TTGGGAAGGGCGGTTTTAAATAAC |
| 20nmsquare23 | 242 | CCGAAAGATGGCGAAAATAGT |
| 20nmsquare24 | 243 | ATTCTGCGTTTAGTTTGGGCCTCTTCGCTATT |
| 20nmsquare25 | 244 | CAGCTCATTTTTGCCCCAAAATGTGAGC |
| 20nmsquare26 | 245 | TCAAAACATTAAAACAGGAAGATT |
| 20nmsquare27 | 246 | GATGGGCGTCGTAAAACTAG |
| 20nmsquare28 | 247 | GTATAAGCAAATGCATTAAACGCGTCTG |
| 20nmsquare29 | 248 | AGCACCTGTAATGTTGATAATCAG |
| 20nmsquare30 | 249 | AACGGTAACATCGTAATATCA |
| 20nmsquare31 | 250 | GAGTAACATTCTCCGTAGGTCACGTTGGTGTA |
| 20nmsquare32 | 251 | GTAGCCAGTAAGAACTATTGTGAATTACCTTATTGACCGTGCCA |
| 20nmsquare33 | 252 | CATGGTACCCCGACTTTTGCGGGAGAAGATTATGACAACAAGAG |
| 20nmsquare34 | 253 | AAAATAACATATTCAATCCAATAGGAACAATGGGATGGGA |
| 20nmsquare35 | 254 | ACAAACGGCGGATGCGATTTCTTT |
| 20nmsquare36 | 255 | CATCAAATAATTTTTTTGTTAAAT |
| 20nmsquare37 | 256 | GGTCATTGCCTGAGTCGGAACCCGAGTCTGG |
| 20nmsquare38 | 257 | GCCGAAAGCTAAATCGGTTGTACCAAAATTTATTTCGTAATGTG |
| 20nmsquare39 | 258 | GCAATAGCAAAATAAGAGGTCATTTTTGTAATTGCTAATAGTAG |
| 20nmsquare40 | 259 | CTGAAACGCAAGGATAAAAATTTTTAGACAGAGCATGAGACAGT |
| 20nmsquare41 | 260 | CTAGCTGATAAATTAATGCAGGGTGAGATGC |
| 20nmsquare42 | 261 | TACTCCTTTTGATTAAGCAATAAA |
| 20nmsquare43 | 262 | GCCTACCAGAATGGCAACTCATATATTTGCACTCCACCGTT |
| 20nmsquare44 | 263 | ACATCCAAGTGCCGGAGCGAG |
| 20nmsquare45 | 264 | CGCTTCTGTAAATCATACAG |
| 20nmsquare46 | 265 | CTCAGGAAGATCTAAATGCAAAAG |
| 20nmsquare47 | 266 | GATTCAAACGGAGAGGGACAGTATCGGC |
| 20nmsquare48 | 267 | CAAATCACATATTCAAGCCAGCTTTCCGGCAC |
| 20nmsquare49 | 268 | CTGAAAAGGTGGCAATATGCATCATCAATTC |
| 20nmsquare- | 269 | AAAAATCAGGTCTTTACCCTGACTA |
| spacer01 | | |
| 20nmsquare- | 270 | CTGTTTAGCTATATTTTCATTTGGG |
| spacer02 | | |
| 20nmsquare- | 271 | TGAGATGGTTTAATTTCAACTTTAA |
| spacer03 | | |
| 20nmsquare- | 272 | CCAGGCAAAGCGCCATTCGCCATTC |
| spacer04 | | |
| 20nmsquare- | 273 | CTCATTATACCAGTCAGGACGTTGG |
| spacer05 | | |
| 20nmsquare- | 274 | GTGCATCTGCCAGTTTGAGGGGACG |
| spacer06 | | |
| 20nmsquare- | 275 | AGCTATTTTTGAGAGATCTACAAAG |
| spacer07 | | |
| 20nmsquare- | 276 | GGGATGTGCTGCAAGGCGATTAAGT |
| spacer08 | | |
| 10nm3merTip-01 | 277 | AAACAGGTCGTCAAACTATCAAAACATTACCA |
| 10nm3merTip-02 | 278 | TCCTAAGCGCCGAAGAAGCTGGAGTGTCTGTA |
| 10nm3merTip-03 | 279 | GCGCACCTAAGAAAAAGAGTAAACAGATTAAAC |
| 10nm3merTip-04 | 280 | GACGGGAGTGGAGAATGTAGCTTTAACAGAAGT |
| 10nm3merTip-05 | 281 | GAGAACCAGCTTATCAGAAAAAAAGTAGTGTT |
| 10nm3merTip-06 | 282 | AACAGTCGGGGTTAATCGTGCCAAGATTTTAT |
| 10nm3merTip-07 | 283 | CTCAGGAAGTCGCAGTAGGCGGAAGTACTGAA |
| 10nm3merTip-08 | 284 | TGGCGAGAAATAAAAGTCTGAAACGTGCCATG |
| 10nm3merTip-09 | 285 | CATGAAACGCAAAGGTCAATATAACTTGAATTA |
| 10nm3merTip-10 | 286 | GCATTAACCGTTGACAGATGTATCACAACCTGAATTAAAA |
| 10nm3merTip-11 | 287 | TCTCTTTAGCCAGCAATATCGGTACATACAAACCAGAAAA |
| 10nm3merTip-12 | 288 | TGGTATCAGTTGTTATAGATATTCACGGCGCTTGTTCACC |
| 10nm3merTip-13 | 289 | CAGCAATCAAGAAGGTATACGAAGGGCGAATAAGTACGCGTGGTtcatctt |
| | | cacactact |
| 10nm3merTip-14 | 290 | GACGGAAGGAGTGAGAGCGCCAACGGCGCATGAACTGTAAtcatcttcaca |
| | | ctact |
| 10nm3merTip-15 | 291 | TGGGAAGCCTTCAATTTAACATCAtcatcttcacactact |
| 10nm3merTip-16 | 292 | TGGTATAGCCAGATGCCCAGCAGTtcatcttcacactact |
| 10nm3merTip-17 | 293 | CGGTACGGCAACGGAATCTTGATTGCGGAGCAtcatcttcacactact |
| 10nm3merTip-18 | 294 | CACGTCCAAATGGTCAGCGTCATAAGAGGGTCTGTTTCAAtcatcttcacact |
| | | act |
| 10nm3merTip-19 | 295 | TATATCAGGCATATAACCCTAAGCTCATtcatcttcacactact |
| 10nm3merTip-20 | 296 | GTCAGGCAAGTTAGTCAAAGAACATCCTtcatcttcacactact |
| 10nm3merTip-21 | 297 | ACCGAAGACATCATGGAAACATCAGACCAGCAAGGAAGCCATAAtcatctt |
| | | cacactact |
| 10nm3merTip-22 | 298 | GGTAGAAGTCGTGTTTTACCTTCTtcatcttcacactact |
| 10nm3merTip-23 | 299 | GATGGCAGCATTTGGCAGGAGGAACAGTATGCAAATTAGCTAATtcatctt |
| | | cacactact |
| 10nm3merTip-24 | 300 | CACGCGGCTTAAACTTGAACGGAAGCGCATAAtcatcttcacactact |
| 10nm3merTip-25 | 301 | ATATCAGCAGACTCAAAAAAAATTTAGGCAGTtcatcttcacactact |
| 10nm3merTip-26 | 302 | ATCTGTCTACAGTAGAGTCACATAtcatcttcacactact |
| 10nm3merTip-27 | 303 | CATACTGACTATATAAACGGTCACATTAACCCCTCACACTtcatcttcacact |
| | | act |
| 10nm3merTip-28 | 304 | AGTTTGAACCACTCCATCTCACCAtcatcttcacactact |
| 10nm3merTip-29 | 305 | CCTTACCAACAGTCTGAATGTAGGGTCGtcatcttcacactact |
| 10nm3merTip-30 | 306 | TGGTCTTTTATCACGAAGTCTAACtcatcttcacactact |
| 10nm3merTip-31 | 307 | cctacgagtcagtcctTCCAGAACACGAACAGGGTCTGTT |
| 10nm3merTip-32 | 308 | cctacgagtcagtcctGCATGAAGATAAGCAGGTCAATAGATGT |
| 10nm3merTip-33 | 309 | cctacgagtcagtcctCTCAGCGGCAAAATTAGCGGCTTG |
| 10nm3merTip-34 | 310 | cctacgagtcagtcctAAATCAATAACAGGCATTGAAGAAAACCTAACGTT |
| 10nm3merTip-35 | 311 | cctacgagtcagtcctATGAGGGAATAGCAAGGAAAGACGCGCCAGCG |
| 10nm3merTip-36 | 312 | cctacgagtcagtcctCGTTTGGTAGATTAGAGGACGGTTAAGAAATA |
| 10nm3merTip-37 | 313 | cctacgagtcagtcctAAATGGTAAAGATGGGACGCTCGGCGCC |
| 10nm3merTip-38 | 314 | cctacgagtcagtcctCAATCACCTTCGCCGCGCTTTAGCGTTACGAACAA |
| 10nm3merTip-39 | 315 | cctacgagtcagtcctAAAATAATCCACTTAAAATACCAT |
| 10nm3merTip-40 | 316 | cctacgagtcagtcctCATCGAACAGAACGGCAACAAATGCTTAAAAGCGG |
| 10nm3merTip-41 | 317 | cctacgagtcagtcctCGGCGTTAATGATTGAAGATTGCGTCCACTGC |
| 10nm3merTip-42 | 318 | cctacgagtcagtcctGGAAACACTTCTTGCATTCCTGAATGAA |
| 10nm4merTip-01 | 319 | ACAGTATTGTTATCGGTAGCAAGCAACTTAAT |
| 10nm4merTip-02 | 320 | CCACTGTTTTAACAGTCGGGAGAGAATATAAC |
| 10nm4merTip-03 | 321 | CAGGTAAACCACCAGTTAGGAACATCCATGCCT |
| 10nm4merTip-04 | 322 | TAACAGGTCTTTATACCATCCTTCAATCACCTT |
| 10nm4merTip-05 | 323 | ACAGAATGCAAGCGCAAGAGTAAAGTAGGCGG |
| 10nm4merTip-06 | 324 | GAATGCCACCGGAGGCGGCTTTTTAACGCGGC |
| 10nm4merTip-07 | 325 | AGAACAGCAACCGCATAAAAGTCTGCAAGAGCA |
| 10nm4merTip-08 | 326 | AATGGCAGGAAACAAAACTAGGGATTTAGCCA |
| 10nm4merTip-09 | 327 | GAAGCAATATTTAATACCAGCATCGAGCCTTG |
| 10nm4merTip-10 | 328 | ATGCGAAAAAAGCATCAGGAACAAACCGCCTCC |
| 10nm4merTip-11 | 329 | AAACTCCTCAGAAAAAAAGTTTGAGTGAGAAC |
| 10nm4merTip-12 | 330 | AAACAATTTAGACATGGCGCCACCACATGATT |
| 10nm4merTip-13 | 331 | CATATCAAGCAAACCTTCAAGGCAGACTtcatcttcacactact |
| 10nm4merTip-14 | 332 | AAATCCGGTCGGCAATAGTTGCATGACCAGCAAGGAAGCCATAAtcatctt |
| | | cacactact |
| 10nm4merTip-15 | 333 | CAACCAAACGTCAACCTTATCAGCTTTAGTAAtcatcttcacactact |
| 10nm4merTip-16 | 334 | GACGGAAGGAGTCATCATCTCAACGGCGAAAAGTAAGAAAtcatcttcacact |
| | | act |
| 10nm4merTip-17 | 335 | AGTTTGTGCATCTCCATCTCACCAtcatcttcacactact |
| 10nm4merTip-18 | 336 | ATCTGAGAGCGCTGATTAAGGCAAtcatcttcacactact |
| 10nm4merTip-19 | 337 | AGAACCCTGAAACGTGCCAAAAAATAGTtcatcttcacactact |
| 10nm4merTip-20 | 338 | AACGGAGCGCATTAAACTTCAGATtcatcttcacactact |
| 10nm4merTip-21 | 339 | ATCATTGTCAGCAGCAATCTGACAAGTAAACTGGCCAGAGtcatcttcacact |
| | | act |
| 10nm4merTip-22 | 340 | AAGCGGAGATGTCAATGTCATTTGCTGCATGAAGTAATCATAACtcatcttc |
| | | acactact |
| 10nm4merTip-23 | 341 | TGCAGACCCATAAAGGACGGTGGTtcatcttcacactact |
| 10nm4merTip-24 | 342 | TCAAATAACAGTCCAAGGCGCTTTGCGAGAAAtcatcttcacactact |
| 10nm4merTip-25 | 343 | AAGGATCCGCGGAGCTCAGGTGATtcatcttcacactact |
| 10nm4merTip-26 | 344 | AAACAGGCAAACATCATAGTCGGAACGTCAATAGTCACACCGGCtcatctt |
| | | cacactact |
| 10nm4merTip-27 | 345 | GTACAGTTAGACAAGCATTACATTTAGTCACCTCACCGAAtcatcttcacact |
| | | act |
| 10nm4merTip-28 | 346 | ACTATCAAAAAAAATTAACCACCAACCGAAGAtcatcttcacactact |
| 10nm4merTip-29 | 347 | CATCCAAAGGATAGCGGTAAGGGGtcatcttcacactact |
| 10nm4merTip-30 | 348 | AAATAATATAACGCCGAAGATTACCAGCtcatcttcacactact |
| 10nm4merTip-31 | 349 | CAAAACAGAGCAGGAGAAATTTCATGAATGAAtcatcttcacactact |
| 10nm4merTip-32 | 350 | GGCGAATAAGTAAGTCATGATAACtcatcttcacactact |
| 10nm4merTip-33 | 351 | AGGTGATACGCGTTCTGCGGTTCCCTATTCCACTGCAACAATTAtcatcttc |
| | | acactact |
| 10nm4merTip-34 | 352 | CTTGGGTCGCCAAGGTACTGCGCGGCGGtcatcttcacactact |
| 10nm4merTip-35 | 353 | CTGACAATCTTTGATTAGCGGTTAtcatcttcacactact |
| 10nm4merTip-36 | 354 | AATTTTGAATCGAACAACCTTATCACGAGTCAACCCCTCAtcatcttcacact |
| | | act |
| 10nm4merTip-37 | 355 | cctacgagtcagtcctGCCACGACCTCATTAGATAACGAGCGCCAGCG |
| 10nm4merTip-38 | 356 | cctacgagtcagtcctAAATGGTAAAGATGGGACGCTCGGCGCC |
| 10nm4merTip-39 | 357 | cctacgagtcagtcctTGGAAAATGTCTGGGATTTTGGGA |
| 10nm4merTip-40 | 358 | cctacgagtcagtcctGACGGAAGATGAGTGACGTCCTTTACCAGCCTCAT |
| 10nm4merTip-41 | 359 | cctacgagtcagtcctTGAAGAAACGTTCTTGGCATAAGCAGCT |
| 10nm4merTip-42 | 360 | cctacgagtcagtcctGCCCTAACGACGTATAAGTCGACC |
| 10nm4merTip-43 | 361 | cctacgagtcagtcctATTAGAAAACACTGTTGAAGCGGCATGGGTGGCAT |
| 10nm4merTip-44 | 362 | cctacgagtcagtcctTCATAGCCTTAGACGATGACCCTCGTCATAAG |
| 10nm4merTip-45 | 363 | cctacgagtcagtcctTCAGTTAAGTGGACGGCAGAACAT |
| 10nm4merTip-46 | 364 | cctacgagtcagtcctACCAAGCACCAAATAGCTGGAGTAACAGTATGGCG |
| 10nm4merTip-47 | 365 | cctacgagtcagtcctTTATTGCCAGTCCTTGCATCATGGCGAC |
| 10nm4merTip-48 | 366 | cctacgagtcagtcctTAATAACCAAATGCAGACGCTCCCCAAACCAT |
| 10nm4merTip-49 | 367 | cctacgagtcagtcctGCTTCGGCGCGTTGACCCAACAGACGAGTGGT |
| 10nm4merTip-50 | 368 | cctacgagtcagtcctTAACCTCAGCGGTAGCTTTATTCG |
| 10nm4merTip-51 | 369 | cctacgagtcagtcctCACTAATCGCGCCCTACCTCTTTAGTCGTAGTGCC |
| 10nm4merTip-52 | 370 | cctacgagtcagtcctACGGTCACACTGAACGCATAATCATGGT |
| 10nm4merTip-53 | 371 | TAGCACCAAGTGCCAGCCTGCAACTATCAGAACTGGAGAC |
| 10nm4merTip-54 | 372 | CAGTAGTGATTGGTATCAGGGTTAAATGCTTAACAGTAGA |
| 10nm4merTip-55 | 373 | AAAACGAAAAAGCACCTTTAGCGTAATATCGGTTTGGTCA |
| 10nm4merTip-56 | 374 | CAGCTTATGGTGTCTGTAAAACAGTGACGATGCAAAAATT |
| 10nm4merTip-57 | 375 | TAGCACCAAGTGCCAGCCTGCAACtagctcactcagtctca |
| 10nm4merTip-58 | 376 | TATCAGAACTGGAGAC |
| 10nm4merTip-59 | 377 | CAGTAGTGATTGGTATCAGGGTTAtagctcactcagtctca |
| 10nm4merTip-60 | 378 | AATGOTTAACAGTAGA |
| 10nm4merTip-61 | 379 | AAAACGAAAAAGCACCTTTAGCGTtaacttcctatctcact |
| 10nm4merTip-62 | 380 | AATATCGGTTTGGTCA |
| 10nm4merTip-63 | 381 | CAGCTTATGGTGTCTGTAAAACAGtaacttcctatctcact |
| 10nm4merTip-64 | 382 | TGACGATGCAAAAATT |
| 10nm4mersp-01 | 383 | ACCAGCAGAGGAAGCATCAGCACCA |
| 10nm4mersp-02 | 384 | GTTTTTGAGATGGCAGCAACGGAAA |
| 10nm4mersp-03 | 385 | ACATACGAAGGCGCATAACGATACC |
| 10nm4mersp-04 | 386 | GGGTCGGCATCAAAAGCAATATCAG |
| 10nm4mersp-05 | 387 | GCAAGATAATCACGAGTATCCTTTC |
| 10nm4mersp-06 | 388 | TTAGCCTCGGTACGGTCAGGCATCC |
| 10nm4mersp-07 | 389 | CACCAGAACGGAAAACATCCTTCAT |
| 10nm4mersp-08 | 390 | ATGTATCCATCTGAATGCAATGAAG |
| 20nm3mertip-01 | 391 | GTTGACCACCTACATACCAAATACGCTCCTGA |
| 20nm3mertip-02 | 392 | CGAGAGTCAGGTACGGCGGTCAGTAGTTTGTTACTCGTCAGAAAATCG |
| | | AA |
| 20nm3mertip-03 | 393 | TGCGCTGGTAATAAGACGACCAGGAATT |
| 20nm3mertip-04 | 394 | GTTAATCGAGCGGCATGGTCAATATTAACACCAAACGTGA |
| 20nm3mertip-05 | 395 | TCTCCTCACTACCATGAACAAAATGTAATCCACGCTCTTT |
| 20nm3mertip-06 | 396 | GGAAGACGGCAGCAATAAACAGTAGTTGAAAGTACA |
| 20nm3mertip-07 | 397 | TAAAAAGTGAGAGCGCGAAGGAGTCAAGGAAG |
| 20nm3mertip-08 | 398 | TAAAATGTCAACAAACCAATCCAAGAGAATCTTCCAAC |
| 20nm3mertip-09 | 399 | ATCATCTTATTAGGGTTAGCCTCGGCATCCACGGCGCTTTGTATCAGG |
| 20nm3mertip-10 | 400 | GCGTCAGCGAAACGCAGTTTTTGACATATCCTACAAAGTCCAAGCA |
| 20nm3mertip-11 | 401 | GTGGAGCTTGCAGACCCATATCGTTCTCTAACCCAG |
| 20nm3mertip-12 | 402 | AGATGAAACCATTTTTGAATCTCTCAGA |
| 20nm3mertip-13 | 403 | CGGTCGTCAGAGTGTCAAAAACGATCATCAAAAGTGGAGG |
| 20nm3mertip-14 | 404 | ATAACTCCAAAGCAGCCTGAAACAAAGTCATTTGGCGAGAAAAGAT |
| 20nm3mertip-15 | 405 | TTGAACACAGAGGGCGCGGCAAAAGTGGTCTA |
| 20nm3mertip-16 | 406 | AGCAGCCTTATGGCCGTCAAGGAGCACATCGG |
| 20nm3mertip-17 | 407 | CAGAAGCCTGAATGAGGGAAACCATAACGAGCATTCAA |
| 20nm3mertip-18 | 408 | TTGAGTTGCGTAGCCATGGCAGCAACCTTAATAGAGGCCAAAGCGGTC |
| | | TG |
| 20nm3mertip-19 | 409 | TTATAGATATCATCTTGATTAAGCGTTAAATCCAAAACGG |
| 20nm3mertip-20 | 410 | GAAACGTATCGGCGTGTGAATCATACCCTCGGCAGCAAGACCTCTAAT |
| 20nm3mertip-21 | 411 | GACTCATGATTTCTTAACCATACTCAGGCACACGAAAA |
| 20nm3mertip-22 | 412 | CCAACTGCGCCGCCAAGGGAAGTACGTG |
| 20nm3mertip-23 | 413 | TAGTCATGGAACCGCACGCAAAGCATGGTCAACGCTACCTGTCGAC |
| 20nm3mertip-24 | 414 | CAATATCACAAAAATACTGATAGCGATTGTTCAGTAACTT |
| 20nm3mertip-25 | 415 | GACTCAGAGACTCATATCTAAACCGAACGTGCCAAGCATAGAAAGGTC |
| 20nm3mertip-26 | 416 | GACAGATTTGTCATTGTGAGTGCGTTCTCTAC |
| 20nm3mertip-27 | 417 | GACCTTTAACTGGCGGCGATTCGTCACAGGTAGCCA |
| 20nm3mertip-28 | 418 | ATCAGAAAATACGCCACGCGCATAACTTTTCC |
| 20nm3mertip-29 | 419 | GACGGTTGACAGTCCCCATTAGCTGTCCTATTAGTGGTTGAACAGCAT |
| | | CG |
| 20nm3mertip-30 | 420 | GCAAAGTAGAGCTGCGCAAGGATACGATTTAATCAAGATA |
| 20nm3mertip-31 | 421 | AAGCATGTCAATGCTCAGTCTCTTTTtcatcttcacactact |
| 20nm3mertip-32 | 422 | TTACCTCCAAATGAAGAATAATTATtcatcttcacactact |
| 20nm3mertip-33 | 423 | GACGGGATACAATTCAGCTCGACGTTTTTGACtcatcttcacactact |
| 20nm3mertip-34 | 424 | GGCGCTGCGTAACCGTCTTCCTCTACGCAATAtcatcttcacactact |
| 20nm3mertip-35 | 425 | TAAGGCCAAAGCGGCTAGGCCACAGCAAGGTCtcatcttcacactact |
| 20nm3mertip-36 | 426 | CAATGGCGCCAGGCAATGGAtcatcttcacactact |
| 20nm3mertip-37 | 427 | CCAGCGCCAGCGATAACCGGATCCACTGCTACtcatcttcacactact |
| 20nm3mertip-38 | 428 | TTAAGCGAGCGCCAGAACGTTTTTTACCGAACGTCACTCAAGTAtcatcttc |
| | | acactact |
| 20nm3mertip-39 | 429 | ACTCGCGACAGCTTGGTTTTTAGTGAGTTTTAGCCATAACCCAGtcatcttc |
| | | acactact |
| 20nm3mertip-40 | 430 | TATCGTGTTTCCCGCAAGGTAAACGCGAGAACATAACCATtcatcttcacact |
| | | act |
| 20nm3mertip-41 | 431 | CTTATCAACAGGGCGTACCAGAGTCAtcatcttcacactact |
| 20nm3mertip-42 | 432 | ACGTTCCAAGAGCTCAAAGAAACGCGGCCATCAACACTCAGTAAtcatctt |
| | | cacactact |
| 20nm3mertip-43 | 433 | AGTAAATTTGAGCAGATTTGAGTAATTCAGTGtcatcttcacactact |
| 20nm3mertip-44 | 434 | TTAATAATGTTTTCCGTAAAGGCCAtcatcttcacactact |
| 20nm3mertip-45 | 435 | ACTTTCATAGCCAGATTAGAGCGCATGAGCAAATTAAACGtcatcttcacact |
| | | act |
| 20nm3mertip-46 | 436 | TCGGGCTTCAATAAACTCTGtcatcttcacactact |
| 20nm3mertip-47 | 437 | TTAACGTATGTTCCATCTTTAGCACTTGGTAAGTTGGATTTCGCtcatcttca |
| | | cactact |
| 20nm3mertip-48 | 438 | GTCAGTTCACAGAATGACACGACCCAGATACAAACTCATCATTAtcatcttc |
| | | acactact |
| 20nm3mertip-49 | 439 | GCTAAAAATTAGGAGCCTGTCGCATTGCAACCAACCCGATGGGCtcatctt |
| | | cacactact |
| 20nm3mertip-50 | 440 | GTCAGTAATTTAGACAGCACGTAAAGGGGCCGAAGCCCCTAATTtcatctt |
| | | cacactact |
| 20nm3mertip-51 | 441 | CAATAGCGAGGGTCCGCCAGCAGTCCACTCGAATTTAGACAGGCtcatct |
| | | tcacactact |
| 20nm3mertip-52 | 442 | CCCTTTGTAGCAATCGCGAAtcatcttcacactact |
| 20nm3mertip-53 | 443 | AAGCTAGAAGTCGTCGGGAGAGGAGTGGACCAGTAGATGAAGTAtcatct |
| | | tcacactact |
| 20nm3mertip-54 | 444 | CAGTTGCGTACCAGGAAGTGCTTCTGtcatcttcacactact |
| 20nm3mertip-55 | 445 | AACCAGAACGTGAAAAAGTGGAAGCtcatcttcacactact |
| 20nm3mertip-56 | 446 | GTCAGTATCAAGTAAACATAAGAGAGAAAACTtcatcttcacactact |
| 20nm3mertip-57 | 447 | GTCGCATAGAAATATAACTGGTAGCTTTCGTATTTTGCGAtcatcttcacact |
| | | act |
| 20nm3mertip-58 | 448 | cctacgagtcagtcctTGGGGATAACATCATGGTGCAT |
| 20nm3mertip-59 | 449 | cctacgagtcagtcctCCTTTCAACGTGAGCCAGGAGGAAGCGGATGTTT |
| 20nm3mertip-60 | 450 | cctacgagtcagtcctGATAATCTGGTTGAACGGCGAAGC |
| 20nm3mertip-61 | 451 | cctacgagtcagtcctACTCTTGTGCCAATTCATCCACGA |
| 20nm3mertip-62 | 452 | cctacgagtcagtcctAGTATAAAGAAATGCCAACGCAAGCCTCCGAGCA |
| 20nm3mertip-63 | 453 | cctacgagtcagtcctTTAATCCTTCTCAGAGCGCATAAAGTGCAATGAA |
| 20nm3mertip-64 | 454 | cctacgagtcagtcctAAGTTGGGCATACATACAACGCCCTGCATACGAAAAG |
| | | ACACGTC |
| 20nm3mertip-65 | 455 | cctacgagtcagtcctCATGCGGCAGACGAGCTTAGTACCTCGCAACGGCTG |
| | | CGGACGAC |
| 20nm3mertip-66 | 456 | cctacgagtcagtcctCGACTTTGAATATTAGACATGCAA |
| 20nm3mertip-67 | 457 | cctacgagtcagtcctTCTCTTTTCATTTTCACATTCTGATTCTGAACAGCTT |
| | | CTTAACG |
| 20nm3mertip-68 | 458 | cctacgagtcagtcctACTGATCAGCATTGGGATTATCATAAAACATACGAC |
| 20nm3mertip-69 | 459 | cctacgagtcagtcctCGTCACGTCCTGCGTGTAGCAA |
| 20nm3mertip-70 | 460 | cctacgagtcagtcctTATCCTTATCATCCTTGATTTCATCGCTGAATAGCA |
| 20nm3mertip-71 | 461 | cctacgagtcagtcctGATGTCTCATTTGAATCGTTAGTTGATGAAGCCACT |
| 20nm3mertip-72 | 462 | cctacgagtcagtcctCAGGTTGGTATCCGAACTGCTTTATTCGTAAACAAG |
| 20nm3mertip-73 | 463 | cctacgagtcagtcctCTGCTGTTAACATGCTTAGGGATTTTATAATAGTTG |
| 20nm3mertip-74 | 464 | cctacgagtcagtcctGAAAGACGAAAAATGTTTCACCATATCCTTCATGAA |
| 20nm3mertip-75 | 465 | cctacgagtcagtcctATAGCAATTCAGCGCCTTTAAG |
| 20nm4mertip-01 | 466 | GTTCAGAAAACTGGCCTAACCACTGCAACAAGAAAA |
| 20nm4mertip-02 | 467 | GCGTCACCTGAAACAATAAGAGGTTTTGAAGAAATAACATCATGGTAAC |
| | | G |
| 20nm4mertip-03 | 468 | AAAGCGGCATAGATATTCAAATAAGTACGGTCAGGCATCC |
| 20nm4mertip-04 | 469 | AGGCGCTGAACGGACTTCATAGAAGCGC |
| 20nm4mertip-05 | 470 | TATAACGTGCTTTAGGTGTCTGTAGAACCAGCAATAAAAG |
| 20nm4mertip-06 | 471 | CTGCATGAATATCAGCACCAACAGATTAGCGGCGTTGACATATCAAAA |
| 20nm4mertip-07 | 472 | ACGGCGCTTTACCAAATACCTAAATAGTTGTTATGGTC |
| 20nm4mertip-08 | 473 | TTAAACTCAAAATTTTACATTAAAATCATGGT |
| 20nm4mertip-09 | 474 | AATATGACGGTAAAGAACCAGTAGTGTCATAGCCAGATGCCCGTCA |
| 20nm4mertip-10 | 475 | ATAACGATACCACTGACCCTAATCACCATGGT |
| 20nm4mertip-11 | 476 | AGAAAAACCGGTCGGACCACCATTACCATCCAAAGGATAAACCCAC |
| 20nm4mertip-12 | 477 | TCTGAATGGGGTCGGCATCAAAAGTAATCACGTTCTTGGT |
| 20nm4mertip-13 | 478 | ACCAAAAACCATTTTTGAATCTCTCAGA |
| 20nm4mertip-14 | 479 | CAGCAAGACAATATCACGAAAATATAATCGGTAACCAACC |
| 20nm4mertip-15 | 480 | CAGTATGCAAATTAGCACAGGCAAAAAATTTACAATGA |
| 20nm4mertip-16 | 481 | CATCACACAGTCCTTGACGGTCGTTCTCTAATCCGC |
| 20nm4mertip-17 | 482 | CTGTCGCACCTCCAGCCGGCAAAAGTGGTCTA |
| 20nm4mertip-18 | 483 | ACTCAGAAGAGAAATAAGCGAACCAAATAAGCAGCTTGCAGACCCATA |
| | | AT |
| 20nm4mertip-19 | 484 | AGCAGCCTTATGGCCGTCAAGGAGCACATCGG |
| 20nm4mertip-20 | 485 | GTCAATAGTAAAGTGCACCGCATGCGGCCATTAGCTGTACACCCTCGG |
| 20nm4mertip-21 | 486 | TACCCTTGAAAGTGGGACGACCAAAAAGTCTCAGGAGGAAGCGGAGC |
| | | AGT |
| 20nm4mertip-22 | 487 | TGCGGTGAAAAAGCGTCCTGAGCGCGCATAAACGTA |
| 20nm4mertip-23 | 488 | CACACAAACTGTAGGAAGTGTCCGGTGGTAGAAGTCGTCA |
| 20nm4mertip-24 | 489 | GATAGGTCGTAGTAATGGATACGCGCGCCGCC |
| 20nm4mertip-25 | 490 | GATAGCGGCGACTGGCAGTCGGCGTGACTGTAACCATAAGGCGAAC |
| 20nm4mertip-26 | 491 | TCTAAACCGAACGTGCCAAGCATAGACAGAATAGCTTCTC |
| 20nm4mertip-27 | 492 | TGGTAAGTTGGATTAAGCACCCCAGCCTCACA |
| 20nm4mertip-28 | 493 | TTTGGCGAGAAAGCTCTTAGGGTCAACGCTACAATACT |
| 20nm4mertip-29 | 494 | TGTTAATTTGAGCAGACTCATTCTAGCT |
| 20nm4mertip-30 | 495 | CCAAATGTTGATTTCTTACCTATTCAGCATCGGACTCAGAGACTCATA |
| 20nm4mertip-31 | 496 | GTTAGCCTCTGAAACAAATGCTTATGGTATCAGGGTTAATGTGGCATT |
| 20nm4mertip-32 | 497 | GTCAAATGAGCCTGACAAGAGAATCTATCGAAATCATCTTCGTGTT |
| 20nm4mertip-33 | 498 | TTTCTTCTGCGTCAGTAAGAACCAGGGCCTTT |
| 20nm4mertip-34 | 499 | GGAAACCATAACGAGCTGGAAACGTACGGATTTAAAAT |
| 20nm4mertip-35 | 500 | ACGGCGTCACCAATCTGCCGAAGCTTGCCTTT |
| 20nm4mertip-36 | 501 | CGCTCTTTGTTCAGTAACTTGACTTTGAGATGGCAGCAAC |
| 20nm4mertip-37 | 502 | AGAGGTTTATGGGATCCAAAGCGGTCATCATCTTGATTAAGCTCATTAG |
| | | G |
| 20nm4mertip-38 | 503 | AACACCATCTTAATCCACTGTTCAATGTCTACGAGAAAGA |
| 20nm4mertip-39 | 504 | CGGCAACATACCAAAGGCGGCTTTACGT |
| 20nm4mertip-40 | 505 | ACTCAGCGGTCAGTAGCAATGTTGACCACCTGCAAT |
| 20nm4mertip-41 | 506 | CAGCAAGGTAGCGACAGTTGTTCCTTGAACGGCGTCGCGTCTGC |
| 20nm4mertip-42 | 507 | AGCACCAAACTTGTTAAATCCCAGCG |
| 20nm4mertip-43 | 508 | GTACTGAATACAAAACTCGGTATAGATAAGCAGGAGAAACAAGC |
| 20nm4mertip-44 | 509 | CAACAGGCGCGAATATCTTTTTGGATTCTTTA |
| 20nm4mertip-45 | 510 | GCGCGAGCGCCAAAATGGTA |
| 20nm4mertip-46 | 511 | TCTTTAATCCAGCAATCACCTCACCAGATACAAACTCATCATTA |
| 20nm4mertip-47 | 512 | GAACATGGGCATATTATCATAAAACGCCCACGCAAAAAGCGGCT |
| 20nm4mertip-48 | 513 | GAAGATTTCACGCGGCGGCAAGTTGCCATCTCTTTAAAGAAGGTCAT |
| 20nm4mertip-49 | 514 | GTACGGGGTGAATCGCAATCTTTTTTAAGTGG |
| 20nm4mertip-50 | 515 | AAGTGACGTTTGAGAGATTACCGCGC |
| 20nm4mertip-51 | 516 | ACGAAGTAGCGGTAAAGTAGCCTCT |
| 20nm4mertip-52 | 517 | CTTCGTCGCAGTCGAACAAGCGGCGCCATCTGTTGACAGG |
| 20nm4mertip-53 | 518 | TTTATAGGGTTTGAATTCATGCGGAGTCAAAG |
| 20nm4mertip-54 | 519 | AGTCATGGCGACCTGGAGTAACAGAAGTACAGGTGCCATAAACA |
| 20nm4mertip-55 | 520 | ACACGAAGGCGGCTCAGCGG |
| 20nm4mertip-56 | 521 | TCCGACAGGAGCAGGAAACGGAGTA |
| 20nm4mertip-57 | 522 | ACTTTCAGCGAAGGCATTTAGTCATGATAAGGACGTGAAA |
| 20nm4mertip-58 | 523 | TTCATGACTTTTTTTAGCCATACTCATCCACAACCAGTAG |
| 20nm4mertip-59 | 524 | CGGGATGAAATGTTTTTTCCATGACCTTCTGCACGTAATTTAGA |
| 20nm4mertip-60 | 525 | GCGACGTGTAGCCACGTATTCGCCAG |
| 20nm4mertip-61 | 526 | GGTTCAATCATTTTTATCGAAAGGTCGCTCAT |
| 20nm4mertip-62 | 527 | ACGCTCGCAAGAGTAAACTTGGTCA |
| 20nm4mertip-63 | 528 | AATTACATAGAAACCAACCACTTCG |
| 20nm4mertip-64 | 529 | GACCCATCAACAGGGACATAAAAAGTAAATAAACGTAAGAAACG |
| 20nm4mertip-65 | 530 | ACGCAACCGGACGCTCGACGCCATTAATACATAATAACATCACTATA |
| 20nm4mertip-66 | 531 | CCTCAACGACTTCTGCCATCAGAATGAGACAG |
| 20nm4mertip-67 | 532 | CGAGGTCAGAAATCCAACGCGTCAGTTTAAGCCACTCAGCGTAC |
| 20nm4mertip-68 | 533 | TGAGTATAATAAATCATAGGCTGAAT |
| 20nm4mertip-69 | 534 | ACCCGATTCTGAACAGCTTCTTGGGAAGTCCATATCATATCTGGGGT |
| 20nm4mertip-70 | 535 | CATTAGCAATGAAAACTCAAAAGTGTTACAGCGACGACAA |
| 20nm4mertip-71 | 536 | CCCTTTGTAGCAGATTTCAT |
| 20nm4mertip-72 | 537 | TCGTCAATCTCAAATTCGTA |
| 20nm4mertip-73 | 538 | GGCGCTGCGTAACCGTCTTCAGTGTCAAATCA |
| 20nm4mertip-74 | 539 | ACGTTCCAAGAGCTCAGAAGCAATACCGAACCTGATCTCAGTAAATC |
| 20nm4mertip-75 | 540 | CATATTTAACCTGACTATTCTTGAATTAAAAA |
| 20nm4mertip-76 | 541 | ACGCCCCTGCAATTAAAATTAGGCCACGAGGA |
| 20nm4mertip-77 | 542 | ACCCTTCCTGAATGAATGGGATAC |
| 20nm4mertip-78 | 543 | ACGCTTGTGCCAATTCATCCACGA |
| 20nm4mertip-79 | 544 | CAGTCAGCCATATAACTGACCA |
| 20nm4mertip-80 | 545 | AGTGGAGGTTGCATTCAAACGATACGTCAGCCAACG |
| 20nm4mertip-81 | 546 | AGCAATAGACCAAACCATCAAT |
| 20nm4mertip-82 | 547 | TTCGCATAGTGCCATGCTACAC |
| 20nm4mertip-83 | 548 | GTCGCAAATGAACCTTCAACGGCAGAAGCTTAAT |
| 20nm4mertip-84 | 549 | AACGCAGACGACACCATTCGGGAGGGTAAAGAAG |
| 20nm4mertip-85 | 550 | AACAAGCAGAATTTTCAAAGTAAGCGTTAGTTGATG |
| 20nm4mertip-86 | 551 | AAGTTGGGCATACATACAACGCCCTGCATACGAAAAGACACGTC |
| 20nm4mertip-87 | 552 | TTTCTTGTTGACAGTCCCAAGCTATTTAATTGCG |
| 20nm4mertip-88 | 553 | CAAAAATTCTAAGCAGTGGCGAGATTATCAGAAAAA |
| 20nm4mertip-89 | 554 | TTCTTGCACAGCAATCAATCACCAGAACGGAAAACATCCTGGAA |
| 20nm4mertip-90 | 555 | CCTAGAGATGTATGACGCGCATGACAAGTTGTCA |
| 20nm4mertip-91 | 556 | CCAACGAAGAAGCAGCATTAACCGTCAATGTATCCA |
| 20nm4mertip-92 | 557 | CTTTGCATTGGGTGAATCATTAGCCTTGTACTCAGG |
| 20nm4mertip-93 | 558 | AGTCTCTCCTCACTACCATGAACAAAATGTAATCCA |
| 20nm4mertip-94 | 559 | ATTTTCTCTCTTTTTGCGTTCGTA |
| 20nm4mertip-95 | 560 | ATAAGACGCATCTCGAACGCAATGAGTAGAGTCAAT |
| 20nm4mertip-96 | 561 | TAACTTTTTCCGTGGAAGCATTTTCATCCCGAAGTTGCGGTTTG |
| 20nm4mertip-97 | 562 | TGCGGACGACGTCAGTCGCAAGGTAAACGCGAACAATTCAACGA |
| 20nm4mertip-98 | 563 | GTTGGAACGTTTTTTACCTTTTTG |
| 20nm4mertip-99 | 564 | ATAACGCGAGGGTATCCTAGCA |
| 20nm4mertip-100 | 565 | AACAGACGATGATTAACAGTCGGGAGAGTGCCAAGA |
| 20nm4mersp-01 | 566 | CCGCTTCGGCGTTATAACCTCACAC |
| 20nm4mersp-02 | 567 | CATGGAAGCGATAAAACTCTGCAGG |
| 20nm4mersp-03 | 568 | TCTTGAACACTCATCCTTAATACCT |
| 20nm4mersp-04 | 569 | CTGCTTTATCAAGATAATTTTTCGA |
| 20nm4mersp-05 | 570 | TTAAGAGGGCGTTCAGCAGCCAGCT |
| 20nm4mersp-06 | 571 | TAGACATAATTTATCCTCAAGTAAG |
| 20nm4mersp-07 | 572 | GTGGTCGGCAGATTGCGATAAACGG |
| 20nm4mersp-08 | 573 | CCAGCAAGGAAGCCAAGATGGGAAA |

TABLE 5
Cholesterol-modified strands for
nucleic acid polygonal nanopore:

| | | |
|---|---|---|
| Chl-1 | SEQ ID NO: 574 | GGACTGACTCGTAGG-chl |
| Chl-2 | SEQ ID NO: 575 | chl-AGTAGTGTGAAGATG |

chl—cholesterol tags

### Example 6

### Assembly and Membrane Insertion of Nucleic Acid Planar Ring Nanopore

The modules were individually prepared via the scaffold-and-staple approach. The design approach was validated by first preparing the surface portion of the polygonal frame (without the perpendicular channel forming tail). The DNA nanostructures were assembled by annealing and cooling.

To obtain membrane pores, the frames were equipped at their bottom with a membrane-spanning partial channel wall, as exemplified with a square pore (FIGS. 9A-9C). The channel wall has the same length as the frame and is composed of a vertical stack of single duplexed DNA duplexes (FIG. 9A). To facilitate the membrane-puncturing of the pores, the frame was also equipped with cholesterol anchors at the bottom of the frame (FIG. 9B). The high membrane affinity of the cholesterol anchors is designed to drive the tip through the membrane (FIG. 9C).

To fold the DNA origami structures, the M13 or PHIX174 (@X174) scaffold (Table 3) and 10× excess of corresponding staples (Table 4) were mixed with the 10× folding buffer to a final concentration of 0.5×TAE pH 8.3 supplemented with 16 mM MgCl₂. The solutions were then heated at 75° C. for 10 min to denature undesired DNA secondary structures and annealed slowly from 65° C. to 25° C. (1 h per° C.), cooled down to 10° C. (5 min per° C.) and kept at 4° C. until collection. After the folding process, the DNA origami structures were purified by excision from 1% agarose gel in 0.5×TBE buffer with 11 mM MgCl₂. Cholesterol-DNA strands (Table 5) were added to the DNA pores at a stoichiometry of 1.5 relative to the number of DNA cholesterol attachment sites prior to use in membrane binding and current recordings experiments.

### Example 7

### TEM Characterization of DNA Origami Pores

DNA nanopores in accordance with an embodiment of the invention were homogenously assembled as shown by single bands in gel electrophoresis as exemplified for square 10 nm pore (FIG. 9D). The designed dimensions and shapes of the polygonal DNA frames were confirmed with transmission electron microscopy and negative staining (FIG. 8).

The pore design was tested with 10 and 20 nm square of blocks featuring bundles of 5× duplexes, and a square tip 4 duplexes deep (FIG. 9A-9C). Each block featured 10 cholesterols for the 10 nm square pore and 15 cholesterols for the 20 nm square pore. The formation of 10 nm square nanopore variant free of cholesterol was confirmed by gel electrophoresis (FIG. 9D). A single band was found implying homogenous assembly. Additional analysis by TEM confirmed the dimensions and geometric shape of the 20 nm square pore (FIG. 9G).

TEM characterisation was conducted according to the following procedure:

6 μL of the purified DNA pores lacking cholesterol was added onto glow discharge-treated TEM grids (Agar Scientific) and stained with 2% uranyl formate solution. TEM analysis was performed on a JEM-2100 electron microscope (JEOL) operated at 200 kV, and the images were acquired with an Orius SC200 camera. Pores carrying cholesterol tags were incubated with POPC SUVs in 0.5×TAE supplemented with 500 mM NaCl for 20 min, followed by deposition onto EM grids, drying, negative staining, and TEM analysis. The preparation of SUVs involved drying a POPC solution (20 mg/mL 50 μL, in chloroform) in a 2 mL glass vial by argon airflow, resuspension of the dried film with 1× incubation buffer (0.5×TAE with 500 mM NaCl to 1 mL) and sonication for 30 min.

### Example 8

### Microscopic Analysis of DNA Pore Binding to GUVs

Membrane binding was validated using pores carrying cholesterols as prepared above. In the assay pores were incubated with small unilamellar vesicles (SUVs). Binding was indicated by an upshift of the of DNA pore's gel electrophoretic band (FIG. 9D) because SUVs are too large to enter the gel meshwork. The smeary pore band in the absence of SUVs (FIG. 9D) may indicate non-specific interaction of some pores by the hydrophobic lipidated faces. Pore binding to SUVs was directly visualized with TEM (FIG. 9H, 20 nm pore). To probe the extent of membrane binding, fluorescence microscopy was used. Pores carrying an Atto647N fluorophore were incubated with giant unilamellar vesicles (GUVs) tagged with Bodipy. Fluorescence microscopy images of GUVs yielded a homogenous fluorescence distribution of both signals suggesting that the pores bound well to membranes (FIG. 9G-9H).

Giant unilamellar vesicles (GUVs) composed of POPC phospholipid were prepared via electroformation. Briefly, two droplets of POPC solution (10 mg/mL in chloroform, 3 μL each) were added onto one ITO glass slide. Evaporation of the solvent in air for 5 min led to the formation of patches of dried lipid film. The glass slide was placed into a Vesicle Prep device (Nanion). The lipid film patches were confined by two O-rings, sucrose solution (1 M in water, 600 μL) was added, and another ITO glass slide was placed on top to form a sealed chamber. An alternating electric field was applied across the two slides (3 V, at 5 Hz) for 120 min, and the solution of vesicles was collected afterwards and stored at 4° C. For DNA pore binding and visualization, 20 μL of 10 nm-wide square pore (˜20 nM) was incubated with 500 μL POPC GUVs solution (fluorescence-labeled with 0.5% β-BODIPY 500/510 C12-HPC, D3793, ThermoFisher) in 1× incubation buffer 50 mM HEPES PH 7.6, 300 mM NaCl for 30 min, and the incubated samples were then imaged with an inverted confocal microscope (SPEinv, Leica).

### Example 9

### Nanopore Recordings

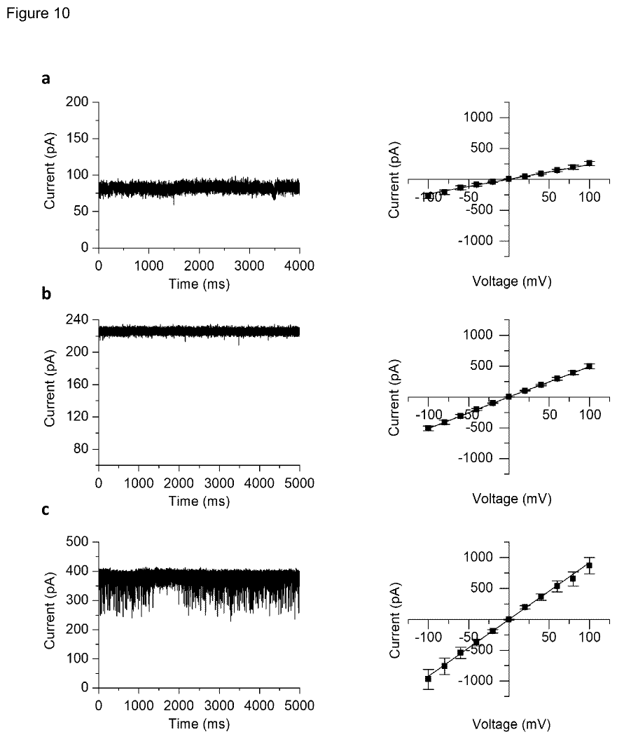

To show that DNA pores puncture the lipid bilayer with a water-filled hole, single-channel current recordings were used. Traces and IV curve of 10 nm triangle DNA nanopore, 10 nm×10 nm square DNA nanopore, 20 nm×20 nm square DNA nanopore are shown in FIG. 10. The conductance of 80 pA, 232 pA and 395 pA are in agreement with the calculated cross-sectional pore area values of 43 nm², 100 nm², and 400 nm².

For planar lipid bilayer electrophysiological current measurements, integrated chip-based, parallel bilayer recording setups (Orbit 16 and Orbit Mini, Nanion Technologies, Munich, Germany) with multielectrode-cavity-array (MECA) chips (IONERA, Freiburg, Germany) were used. Bilayers were formed by painting of DPhPC dissolved in octane (10 mg mL-1). The electrolyte solution was 1 M KCl and 10 mM HEPES, pH 8.0. For pore insertion, a 2:1 mixture of cholesterol-anchored DNA nanopores and 0.5% OPOE (n-octyloligooxyethylene, in 1 M KCl, 10 mM HEPES, pH 8.0) was added to the cis side of the bilayer. Successful incorporation was observed by detecting current steps. The Orbit 16 current traces were Bessel-filtered at 2.873 kHz and acquired at 10 KHz with an EPC-10 patch-clamp amplifier (HEKA Elektronik, Lambrecht/Pfalz, Germany) applying the PATCHMASTER software (HEKA Elektronik). The Orbit Mini current traces were not Bessel-filtered and acquired at 10 KHz, using Element Data Recorder software (Element s.r.l., Italy). Single-channel analysis was performed using Clampfit (Molecular Devices, Sunnyvale, CA, USA).

The nanopore structures of the present invention provided the following advantages:

### 1) Large Lumen.

It allows the passage of bigger analytes like proteins, while the existing origami pores only have smaller width.

### 2) Efficient Use of Materials.

For a conventional origami pore, it usually consumes hundreds of staples (˜8000 bp), while in this design, only ten strands (300 bp) were needed to form the structure.

### 3) Simple and Fast Preparation Conditions.

For a conventional origami pore, it takes several days to fold the origami structure, while folding was shortened to 2 hours in this design.

### 4) Easy and Precise Functionalization.

The well-defined formation of the pore structure conferred upon it atom-level accuracy, this allows for further engineering to acquire a wide range of functionalities.

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid sequence(s) is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1.-29. (canceled)

30. A membrane into which is inserted at least one membrane-spanning nanopore: wherein
the membrane-spanning nanopore comprises:
one or more polynucleotide strands that provide a scaffold component; and
a plurality of polynucleotide strands that provide a plurality of staple components:
wherein each of the plurality of staple components hybridise to the scaffold component:
wherein the orientation of a major portion of at least one polynucleotide strand comprised within the scaffold component is substantially parallel to a planar surface of a membrane:
wherein the nanopore defines a channel that is suitable for perforating the membrane:
the channel having a longitudinal axis extending along the centre thereof and a minimum internal dimension perpendicular to the longitudinal axis of at least about 3 nm.

31. The membrane of claim 30, wherein the membrane is selected from the group consisting of: a membrane comprising a semi-fluid membrane; and a solid state membrane.

32. The membrane of claim 31, wherein the semi-fluid membrane is composed of amphiphilic synthetic block copolymers, suitably selected from hydrophilic copolymer blocks and hydrophobic copolymer blocks.

33. The membrane of claim 31, wherein the membrane is in the form of a vesicle, a micelle, a planar membrane, or a droplet.

34. The membrane of claim 31, wherein the solid state membrane is formed of a material selected from the group consisting of:
Group II-IV and III-V oxides and nitrides, solid state organic and inorganic polymers, plastics, elastomers, and glasses.

35. A biological sensor, wherein the biological sensor comprises the membrane of claim 31 and electrical measurement apparatus.

36. The biological sensor of claim 35, wherein the biological sensor further comprises a fluorescence measurement apparatus.

37. A membrane-spanning nanopore, wherein the nanopore is configured to form an annular or polygonal structure that is located within a membrane, the nanopore comprising:
i) at least one scaffold polynucleotide strand; and
ii) a plurality of staple polynucleotide strands:
wherein each of the plurality of staple polynucleotide strands hybridises to the at least one scaffold polynucleotide strand to form a three-dimensional configuration of the membrane-spanning nanopore,
and wherein the membrane-spanning nanopore is positioned such that the orientation of a majority of the at least one scaffold polynucleotide strand is substantially parallel to the planar surface of the membrane and thereby defines a central channel with a minimum internal width of at least about 3 nm.

38. The membrane-spanning nanopore of claim 37, wherein either or both of the at least one scaffold polynucleotide strand and the plurality of staple polynucleotide strands further comprises a plurality of hydrophobic anchor molecules, wherein the at least one hydrophobic anchor facilitates insertion of the nanopore into the membrane.

39. The membrane-spanning nanopore of claim 37, wherein assembly of the nanopore and/or components thereof is via DNA origami techniques.

40. The membrane-spanning nanopore of claim 38, wherein the plurality of hydrophobic anchor molecules is comprised of a hydrophobic portion of a polynucleotide strand comprised within the scaffold component.

41. The membrane-spanning nanopore of claim 40, wherein the plurality of hydrophobic anchor molecules is comprised of at least one of the plurality of staple polynucleotide strands or a hydrophobic portion thereof.

42. The membrane spanning nanopore of claim 40, wherein the plurality of hydrophobic anchor molecules are:
a) attached to and spaced substantially equidistantly about the periphery of the nanopore, and wherein the plurality of hydrophobic anchor molecules are orientated radially outwardly from the longitudinal axis of the channel of the nanopore; and/or
b) attached to a membrane-facing side of the nanopore or a portion thereof such that the plurality of hydrophobic anchor molecules are orientated to interact with and/or extend into the membrane once inserted.

43. The membrane spanning nanopore of claim 41, wherein the nanopore comprises at least four hydrophobic anchor molecules.

44. The membrane spanning nanopore of claim 38, wherein the at least one hydrophobic anchor molecule is selected from the group consisting of: a lipid; and a porphyrin.

45. The membrane spanning nanopore of claim 44, wherein the lipid is selected from the group consisting of:
sterols, alkylated phenols; flavones, saturated and unsaturated fatty acids, and synthetic lipid molecules.

46. The membrane spanning nanopore of claim 37, wherein the nanopore is in the form of a polygon selected from the group consisting of:
a triangle, a square, a quadrilateral, a pentagon, a hexagon, a heptagon, and an octagon.

47. The membrane spanning nanopore of claim 37, wherein the minimum internal width of the channel of the nanopore is less than about 20 nm.

48. The membrane spanning nanopore of claim 37, wherein the scaffold component comprises a polynucleotide having a sequence selected from the group consisting of:
SEQ ID NO: 1, SEQ ID NO: 2, SEQ ID NO: 13, and SEQ ID NO: 14.

49. The membrane spanning nanopore of claim 37, wherein the plurality of staple components comprise at least one polynucleotide having a sequence selected from one or more of the group consisting of:
SEQ ID NOs: 3 to 12, SEQ ID NOs: 15 to 56, SEQ ID NOs: 65 to 94, SEQ ID NOs: 105 to 140, and SEQ ID NOs: 153 to 268.
